(19) United States
(12) Patent Application Publication
(10) Pub. No.: US 2021/0290310 A1
(43) Pub. Date: Sep. 23, 2021

(54) HYBRID-DIMENSIONAL, AUGMENTED REALITY, AND/OR REGISTRATION OF USER INTERFACE AND SIMULATION SYSTEMS FOR ROBOTIC CATHETERS AND OTHER USES

(71) Applicant: Project Moray, Inc., Belmont, CA (US)

(72) Inventors: Keith Phillip Laby, Oakland, CA (US); Augie R. Maddox, San Francisco, CA (US); Mark D. Barrish, Belmont, CA (US); Miles D. Alexander, Fremont, CA (US); Boris M. Preising, Palo Alto, CA (US)

(73) Assignee: Project Moray, Inc., Belmont, CA (US)

(21) Appl. No.: 17/340,773

(22) Filed: Jun. 7, 2021

Related U.S. Application Data
(63) Continuation of application No. PCT/US2019/065752, filed on Dec. 11, 2019.
(60) Provisional application No. 62/778,148, filed on Dec. 11, 2018.
(60) Provisional application No. 62/896,381, filed on Sep. 5, 2019.
(60) Provisional application No. 62/905,243, filed on Sep. 24, 2019.

(51) Int. Cl. A61B 34/10; A61B 34/30; A61B 90/00

(52) U.S. Cl. CPC A61B 34/10; A61B 34/30; A61B 90/36; A61B 2034/107; A61B 2090/365; A61B 2090/376; A61B 2090/378; A61B 2034/301

(57) ABSTRACT

Devices, systems, and methods are provided for user input to control automated movement of catheters and other elongate bodies. Fluid drive systems can be used to provide robotically coordinated motion. Precise control over actual robotic catheter-supported tools are enhanced by moving a virtual version of the tool from a starting location of an actual tool to a desired ending position and orientation. A processor of the system can then generate synchronized actuator drive signals to move the tool without following the (often meandering) path input by the system user. The progress of the tool along a multi-degree-of-freedom trajectory can be controlled with a simple 1D input. Standard planar or proprietary input devices can be used for orientation and translation movements. Hybrid image display with 2D and 3D components is provided, along with spacial constrained movement to workspace boundaries.

### CROSS REFERENCE TO RELATED APPLICATIONS

The present application is a continuation under 35 U.S.C. 111(a) of PCT International Application No. PCT/US2019/065752, filed on Dec. 11, 2019, which claims the benefit of and priority to U.S. Provisional Application Ser. No. 62/778,148 filed on Dec. 11, 2018, 62/896,381 filed Sep. 5, 2019, and 62/905,243 filed Sep. 24, 2019. These applications are incorporated by reference herein in their entirety for all purposes.

### FIELD OF THE INVENTION

In general, the present invention provides improved devices, systems, and methods for using, training for the use of, planning for the use of, and/or simulating the use of elongate articulate bodies and other tools such as catheters, borescopes, continuum robotic manipulators, rigid endoscopic robotic manipulators, and the like. In exemplary embodiments, the invention provides in situ robotic catheter motion planning, and linear catheter position control over complex trajectories, particularly for catheter systems driven by fluid pressure.

### BACKGROUND OF THE INVENTION

Diagnosing and treating disease often involve accessing internal tissues of the human body, and open surgery is often the most straightforward approach for gaining access to internal tissues. Although open surgical techniques have been highly successful, they can impose significant trauma to collateral tissues.

To help avoid the trauma associated with open surgery, a number of minimally invasive surgical access and treatment technologies have been developed, including elongate flexible catheter structures that can be advanced along the network of blood vessel lumens extending throughout the body. While generally limiting trauma to the patient, catheter-based endoluminal therapies can be very challenging, in-part due to the difficulty in accessing (and aligning with) a target tissue using an instrument traversing tortuous vasculature. Alternative minimally invasive surgical technologies include robotic surgery, and robotic systems for manipulation of flexible catheter bodies from outside the patient have also previously been proposed. Some of those prior robotic catheter systems have met with challenges, possibly because of the difficulties in effectively integrating large and complex robotic pull-wire catheter systems into the practice of interventional cardiology as it is currently performed in clinical catheter labs. While the potential improvements to surgical accuracy make these efforts alluring, the capital equipment costs and overall burden to the healthcare system of these large, specialized systems is also a concern. Examples of prior robotic disadvantages that would be beneficial to avoid may include longer setup and overall procedure times, deleterious changes in operative modality (such as a decrease in effective tactile feedback when initially accessing or advancing tools toward an internal treatment site), and the like.

A new technology for controlling the shape of catheters has recently been proposed which may present significant advantages over pull-wires and other known catheter articulation systems. As more fully explained in US Patent Publication No. US 2016/0279388, entitled “Articulation Systems, Devices, and Methods for Catheters and Other Uses,” published on Sep. 29, 2016 (assigned to the assignee of the subject application and the full disclosure of which is incorporated herein by reference), an articulation balloon array can include subsets of balloons that can be inflated to selectively bend, elongate, or stiffen segments of a catheter. These articulation systems can direct pressure from a simple fluid source (such as a pre-pressurized canister) toward a subset of articulation balloons disposed along segment(s) of the catheter inside the patient so as to induce a desired change in shape. These new technologies may provide catheter control beyond what was previously available, often without having to resort to a complex robotic gantry, without having to rely on pull-wires, and even without having the expense of electric motors. Hence, these new fluid-driven catheter systems appear to provide significant advantages.

Along with the advantages of fluid-driven technologies, significant work is now underway on improved imaging for use by interventional and other doctors in guiding the movement of articulated therapy delivery systems within a patient. Ultrasound and fluoroscopy systems often acquire planar images (in some cases on different planes at angularly offset orientations), and news three-dimensional (3D) imaging technologies have been (and are still being) developed and used to show these 3D images. While 3D imaging has some advantages, guiding interventional procedures with reference to 2D images (and other uses) may still have benefits over at least some of the new 3D imaging and display techniques, including the ability to provide alignment with target tissues using quantitative and qualitative planar positioning guidelines that have been developed over the years.

Despite the advantages of the newly proposed fluid-driven robotic catheter and imaging systems, as with all successes, still further improvements and alternatives would be desirable. In general, it would be beneficial to provide further improved medical devices, systems, and methods, as well as to provide alternative devices, systems, and methods for users to input, view, and control the automated movements thereof. For example, the position and morphology of a diseased heart tissue relating to a structural heart therapy may change significantly between the day on which diagnostic and treatment planning images are obtained, and the day and time an interventional cardiologist begins deploying a therapy within the beating heart. These changes may limit the value of treatment planning prior to start of an interventional procedure. However, excessive engagement of structural heart devices against sensitive tissues of the heart, as might be imposed when attempting a multiple alternative trajectories for advancing a structural heart tool from an access pathway to a target position, could induce arrhythmias and other trauma. Hence, technologies which facilitate precise movements of these tools and/or in situ trajectory planning for at least a portion of the overall tool movement, ideally while the tool is near or in the target treatment site, would be particularly beneficial. Improved display systems that provided some or all of the benefits of both 2D and 3D imaging would also be beneficial.

### BRIEF SUMMARY OF THE INVENTION

The present invention generally provides improved devices, systems, and methods for using, training for the use of, planning for the use of, and/or simulating the use of elongate bodies and other tools such as catheters, borescopes, continuum robotic manipulators, rigid endoscopic robotic manipulators, and the like. The technologies described herein can facilitate precise control over both actual and virtual catheter-based therapies by, for example, allowing medical professionals to plan an automated movement of a therapeutic tool supported by the catheter based on a starting location of the catheter previously inserted into the heart. Optionally, a virtual version of the tool can be safely moved from that starting location along a meandering path through a number of different locations till the user has identified a desired ending position and orientation of the tool for the movement. A processor of the system can then generate synchronized actuator drive signals to move the tool in the chamber of the heart from its starting point to the end without following the meandering input path, with the progress of the tool along its trajectory being under the full control of the user, such as with a simple linear input that allows the user to advance or retract along a desired fraction of the trajectory. Alternative actual and/or virtual robotic systems facilitate the use of standard input devices that accommodate at least two-dimensional or planar input (such as a mouse, tablet, phone, or the like) to re-position elongate bodies such as catheters, optionally using separate but intuitive input modes for orientation and translation movements. Still further aspects provide hybrid display formats which can take advantage of a combination of 2D image components and 3D image components in an overall 3D display space to enhance 3D situational awareness, often by combining at least one (often multiple) 2D tissue image in a 3D display space that also includes a 3D model of an instrument that can be seen in the tissue images, optionally with 2D virtual models superimposed on the tissues and instrument of the image plane. The image may optionally be shown on a 2D (such as a screen) or 3D display modalities (such as 3D stereoscopic screens, Augmented Reality (AR) or virtual Reality (VR) glasses, or the like). Input systems that facilitate driving of catheters and other articulate bodies relative to 2D image planes, such as by keeping the tip or a tool receptacle within the field of view of an ultrasound image plane, are also provided.

In a first aspect, the invention provides an image-guided therapy method for treating a patient body. The method comprises generating a three-dimensional (3D) virtual therapy workspace inside the patient body and a three-dimensional (3D) virtual image of a therapy tool within the 3D virtual workspace. An actual 2D image of the tool in the patient body is aligned with the 3D virtual image, the actual image having an image plane. The actual image is superimposed with the 3D virtual image so as to generate a hybrid image, and the hybrid image is transmitted to a display having a display plane so as to present the hybrid image with the image plane of the actual image at an angle relative to the display plane, for example, with the actual planar image shown so as to appear at an offset angle to the plane of the display.

In optional aspects, the generating, aligning, superimposing, and tramsitting may be performed by a processor by manipulating image data, with the processor typically being included in an imaging and/or therapy delivery system (ideally being included in a robotic catheter system). A number of additional aspects of the method (and corresponding apparatus) are described herein, with the aspects often being independent (so as to stand on their own merits as stand-alone methods or systems with or without the image guided methodology described immediately above), but also being suitable to be used together.

For example, in another aspect the invention provides an image-guided therapy system for use with a tool movable in an internal surgical site. An image capture device is included in the system for acquiring an actual image encompassing the tool and a target tissue and having an image plane, and a display is also provided for displaying the actual image. The system comprises a simulation module configured to generate a three-dimensional (3D) virtual workspace and a virtual three-dimensional (3D) image of the tool within the 3D virtual workspace. A registration module is configured align the actual image with the 3D virtual image. The simulation module is configured to superimpose the actual image with the 3D virtual image so as to transmit a hybrid image including the 3D virtual workspace and the image plane of the actual image at an angle relative to the display.

In an optional aspect, the image acquisition system employed with the systems and methods described herein may including an ultrasound imaging system for generating a plurality of planar images having first and second image planes. The simulation system can be configured to offset the first and second image planes from the virtual tool in the 3D virtual workspace and to superimpose a 2D virtual image of the tool on the first and second image planes in the hybrid image.

In another aspect, the invention provides a method for aligning a therapeutic or diagnostic tool with a target tissue adjacent an internal site in a patient. The method makes use of an elongate body inserted into the patient, the elongate body having a receptacle to support the tool. The receptacle defines a first pose within the internal surgical site. The method comprises receiving, with a processor of a surgical robotic system and from a user, input for moving the receptacle (or an image thereof) from the first pose to a second pose within the internal surgical site. The input optionally defines an intermediate input pose after the first pose and before the second pose. The processor also receives a movement command to move the receptacle, and in response, transmits drive signals to a plurality of actuators so as to advance the receptacle along a trajectory from the first pose toward the second pose, with the trajectory optionally being independent of the intermediate input pose.

In an optional aspect, the movement command received by the processor comprises a command to move along an incomplete spatial portion of a trajectory from the first pose to the second pose and to stop at an intermediate pose between the first pose and the second pose. In response, to the movement command, the processor transmits drive signals to a plurality of actuators coupled with the elongate body so as to move the receptacle toward the intermediate pose.

In another aspect, the invention provides a system for aligning a therapeutic or diagnostic tool with a target tissue adjacent an internal site in a patient. The system comprises an elongate body having a proximal end and a distal end with an axis therebetween. The body has a receptacle configured to support the tool within the internal surgical site so that the tool defines a first pose. A plurality of actuators is driving couplable with the elongate body so as to move the receptacle within the surgical site. A processor is couplable with the actuators. The processor has a first module and a second module. The first module is configured to receive input from a user for moving the receptacle (or an image thereof) from the first pose to a second pose within the internal surgical site. The input optionally defines an intermediate input pose between the first pose and the second pose. The second module is configured to receive a movement command, and in response, to drive the actuators so as to move the receptacle along a trajectory from the first pose to the second pose, with the trajectory optionally being independent of the intermediate input pose. Typically, the input defines an input trajectory between the first pose and the second pose, and the intermediate input pose is disposed along the input trajectory. The plurality of actuators can optionally be energized so that the elongate body disregards the input trajectory as the receptacle moves along the trajectory, so that the receptacle may not be driven to (or even toward) the intermediate input pose. This can, for example, allow a user to evaluate a series of candidate tool poses and/or trajectories in silico without imposing the trauma of actually moving the tool to unsuitable configurations, all from the actual starting location and orientation of the tool in or near the heart.

In another optional aspect, the first module can optionally be configured to receive, from a user after the receptacle is in the first pose, a second pose. The second module is configured to receive, from the user, a movement command and, in response, to drive the actuators so as to move the receptacle along an incomplete spatial portion of a trajectory from the first pose to the second pose and stop at an intermediate pose between the first pose and the second pose.

Optional and independent features may be included to enhance the functionality of the devices described herein. For example, the processor may be configured to calculate the trajectory from the first pose to the second pose, and a series of intermediate poses of the receptacle along the trajectory between the first pose and the second pose. The processor, when in the second mode, may be configured to receive a series of additional movement commands, and in response, to drive the actuators so as to move the receptacle with a plurality of incremental movements along a series of incomplete portions of the trajectory between the intermediate poses. These movement commands may also induce the receptacle to stop at one or more of the intermediate poses. Advantageously, the additional movement commands can include a move back command. The processor, in response to the move back command, can be configured to drive the actuators so as to move the receptacle along the trajectory away from the second pose and toward the first pose.

Optionally, the processor can be configured so as to receive the movement command as a one-dimensional input signal corresponding to a portion of the trajectory. Therein the processor can be configured to energize the plurality of actuators so as to move the receptacle in a plurality of degrees of freedom of the elongate body along the trajectory. Such an arrangement provides a simple and intuitive control that keeps the movement speed and advancement under full control of the user, allowing the user to concentrate on the progress of the movement and the relationship of the tool to adjacent tissue, rather than being distracted by having to enter a complex series of multidimensional inputs that might otherwise be needed to follow a complex trajectory.

Optionally, an intra-procedure image capture system can be oriented to image tissue adjacent the internal surgical site so as to generate image data. A display can be coupled to the image capture system so as to show, in response to the image data, an image of the adjacent tissue and the tool in the first pose (and/or in other poses). An input device can be coupled with the processor and disposed to facilitate entry of the input by the user with reference to the image of the adjacent tissue and the tool as shown by the display. The processor can have a simulation module configured to superimpose a graphical tool indicator with the image of the adjacent tissue in the display. A pose of the tool indicator can move with the input so as to facilitate aligning the second pose with the target tissue. Hence, the image can comprise a calculated pose of the tool indicator relative to the target tissue. The processor can have a simulation input mode in which the processor energizes the actuators so as to maintain the first pose of the tool when the user is entering the input for the second pose. This arrangement facilitates evaluation of candidate poses using a virtual or simulated tool, with the tool indicator often comprising a graphical model of the tool and at least some of the supporting catheter structure.

Optionally, the processor has a master-slave mode in which the processor energizes the actuators to move the receptacle toward the second pose while the user is entering the input for the second pose. Preferably, the processor has both a simulation mode and a master-slave mode to facilitate alignment of tools with target tissues using both a graphical tool indicator (during a portion of the procedure) and real-time or near real-time moving images of the actual tool.

Optionally, the system includes a two-dimensional input device couplable to the processor. The processor may have a first mode configured to define a position of the receptacle relative to the adjacent tissue. The processor may optionally also have a second mode configured to define an orientation of the receptacle relative to the adjacent tissue. The processor may (or may not) also have a third mode configured to manipulate an orientation of the adjacent tissue as shown in the display.

Preferably, the elongate body comprises a flexible catheter body configured to be bent proximally of the receptacle by the actuators. The actuators may comprise fluid-expandable bodies disposed along the elongate body, and a fluid supply system can couple the processor to the actuators. The fluid system can be configured to transmit fluid along channels of the elongate body to the actuators.

In another aspect, the invention provides a robotic catheter system for aligning a therapeutic or diagnostic tool with a target tissue by an internal surgical site in a patient. The system comprises an elongate flexible catheter body configured to be inserted distally into the internal surgical site. The tool is supportable adjacent a distal end of the elongate body to define a first pose within the internal surgical site. A plurality of actuators are couplable to the elongate body. A processor is couplable to the actuators and configured to i) receive a desired second position of the tool within the internal surgical site, ii) calculate a tool trajectory of the tool from the first position to the second position (along with associated drive signals for the actuators to move the elongate body along a tool trajectory from the first position to the seconded position), iii) receive an input signal with a single degree of freedom defining a desired portion of the trajectory, and iv) drive the actuators so as to move the tool along the portion of the trajectory defined by the input signal, the portion having a plurality of degrees of freedom.

In yet another aspect, the invention provides a system for manipulating a real and/or virtual elongate tool in a three-dimensional workspace. The tool has an axis, and the system comprises an input/output (I/O) system configured for showing an image of the tool and for receiving a two-dimensional input from a user, the I/O system having a plane and the axis of the tool as shown in the tool image having a display slope along the plane, a first component of the input being defined parallel to a first axis corresponding to the tool display slope, a second component of the input being defined along a second axis of the input plane perpendicular to the tool display slope. A processor is coupled to the I/O system, the processor having a translation mode and an orientation mode. The processor in the orientation mode is configured to, in response to the first component of the input, induce rotation of the tool in the three-dimensional workspace about a first rotational axis. The first rotational axis is parallel to the plane and perpendicular to the tool axis. The orientation mode also has the processor configured to, in response to the second component of the input, induce rotation of the tool image about a second rotational axis, the second rotational axis perpendicular to the tool axis and the first rotational axis.

Optionally, the first rotational axis and the second rotational axis intersect with the tool axis at a center of rotation. The processor may be configured to superimpose, with the image of the tool, a spherical rotation indicator concentric with the center of rotation. Rotation indicia may be included with the spherical rotation indicator, the rotation indicia rotating about the center of rotation with the input so that movement of the indicia displayed adjacent the user move in an orientation corresponding with an orientation of the input. The rotation indicia may encircle the axis along a first side of the spherical rotation indicator toward the user from the center of rotation at a start of a rotation. The rotation indicia may rotate with the tool in the three-dimensional space so that the rotation indicia remain on the first side of the spherical rotation indicator during the rotation, and the processor, when a second side of the spherical rotation indicator opposite the first side is toward the user after the rotation, may reposition the rotation indicia to the second side. The processor, when in the translation mode, can optionally be configured to translate the tool along the first rotational axis in response to the first input component and along the second rotational axis in response to the second input component. The processor may, in response to the tool axis being within an angle range of normal to the imaging plane, align the first and second axes with the lateral display axis and the transverse display axis, respectively, the angle being between 5 and 45 degrees.

In another aspect, the invention provides a system for aligning a therapeutic or diagnostic tool with a target tissue adjacent an internal site in a patient. The system comprises an elongate body having a proximal end and a distal end with an axis therebetween. The body has a receptacle configured to support the tool within the internal surgical site so that the tool defines a first pose. A plurality of actuators are driving couplable with the elongate body so as to move the receptacle within the surgical site with a plurality of degrees of freedom. A processor is couplable with the actuators and configured to receive input from a user for moving the receptacle from the first pose to a second pose within the internal surgical site. A remote image capture system is oriented toward the internal surgical site and configured to acquire an image of the target through tissue of the patient. The processor is configured to constrain the tool to movement adjacent a plane by coordinating articulation about the degrees of freedom.

In another aspect, the invention provides a medical robotic simulation system for use with a computer coupled an input device. The system comprises tangible media embodying machine-readable code with instructions for displaying, on a display, an image of an elongate flexible body. The body has a proximal end, a distal end, and a tool receptacle configured to support a therapeutic or diagnostic tool in alignment with a target tissue adjacent an internal surgical site. The instructions are also for receiving, with the input device, a movement command from a user. The movement command is for moving the receptacle from a first pose toward a second pose that is aligned with the target tissue within the internal surgical site. The instructions are also for transmitting at least two-dimensional input in response to the movement command and from the input device to the computer, and for determining, with the computer and in response to the input, articulation of the body so as to induce the receptacle to move toward the second pose. The instructions can also result in displaying, on the display, of the determined articulation of the body and movement.

Optionally, the computer comprises an off-the-shelf computer couplable with a cloud and the input device comprises an off-the-shelf device having a sensor system configured for measuring changes in position with at least two degrees of freedom. The body may comprise a virtual flexible body, facilitating use of the system for planning, training, therapeutic tool evaluation, and/or the like. The system may also comprise an actual robotic system (in addition to or instead of being capable of virtual movements), with the system including an actual elongate body having an actual proximal end and an actual distal end with an actual receptacle configured for supporting an actual therapeutic or diagnostic tool. A plurality of actuators will typically be coupled with the elongate body, and an actual drive system can be couplable with the cloud and/or with the actuators so as to induce movement of the receptacle within an actual internal surgical site in a patient. A clinical input device having a clinical sensor system can be configured for measuring changes in position with the at least two degrees of freedom of the off-the-shelf device to allow the user to transition easily between the virtual and actual components of the system. Coupling of the virtual and actual components via the cloud facilitate analytic data tracking, coordinated updates of both systems to accommodate new and revised elongate body and therapeutic tool designs, improvements in the user interface, and the like.

In another aspect, the invention provides a method for presenting an image to a user of a target tissue of a patient body. The method comprises receiving a first two-dimensional (2D) image dataset, the first 2D dataset defining a first image including the target tissue and a tool receptacle of a tool delivery system disposed within the patient body. The first image has a first orientation relative to the receptacle. A second 2D image dataset defining a second target image including the target tissue and the tool delivery system is also received, the second image having a second orientation relative to the receptacle, the second orientation being angularly offset from the first orientation. Hybrid 2D/three-dimensional (3D) image data is transmitted to a display device so as to present a hybrid 2D/3D image for reference by the user. The hybrid image includes the first 2D image with the first orientation relative to a 3D model of the tool delivery system, and the second 2D image having the second orientation relative to the 3D model, the first and second 2D images positionally offset from the model.

Preferably, the hybrid image also includes a 3D virtual image of the model, the model comprising a calculated virtual pose of the receptacle. The first 2D image can be disposed on a first plane in the hybrid image, the first plane being offset from the model along a first normal to the first plane; and/or the second 2D image may be disposed on a second plane in the hybrid image, the second plane being offset from the model along a second normal to the second plane. With or without such a 3D model image, the hybrid image may include a first 2D virtual image of the model superimposed on the first 2D image, the first 2D virtual image being at the first orientation relative to the model; and/or the hybrid image may include a second 2D virtual image of the model superimposed on the second 2D image, the second 2D virtual image being at the second orientation relative to the model. These 2D virtual images may comprise planar images of the model (including one, some, or all of the tip, receptacle, tool, articulated body, etc.) projected onto the image data planes. As the image data planes typically also include images of both tissue and the actual tool etc., these superimposed planar images facilitate user or automated verification of alignment of the virtual model with the actual articulated device, of the movement of the articulated device relative to the tissue, and the like.

Preferably, the model includes a phantom defining a phantom receptacle pose angularly and/or positionally offset from the virtual receptacle pose. The 3D virtual image includes an image of the phantom, and the hybrid image includes a first 2D augmented image showing the phantom with the first orientation superimposed on the first 2D image, and a second 2D augmented image of the phantom with the second orientation superimposed on the second 2D image. Optionally, the method further comprises receiving a movement command from a hand of the user to move relative to the display, and moving he phantom pose in correlation with the movement command. The moved phantom can be displayed on the first 2D image and the second 2D image. A trajectory can be calculated between the virtual tool and the phantom and the tool can be moved within the patient body by articulating an elongate body supporting the tool in response to a one-dimensional (1D) input from the user.

Independently, the device and methods described herein may involve constraining motion of the receptacle, tool, tip, or the like relative to the first plane so that an image of the receptacle (for example) moves along the first plane, or normal to the first plane.

Optionally, the first 2D image comprises a sufficiently real-time video image for safe therapy based on that image (typically having a lag of less than 1 second). The second 2D image may comprise a recorded image (optionally being a series of recorded images, such as those included in a brief video loop) of the target tissue and the actual tool system. The first and second 2D images may comprise ultrasound, fluoroscope, magnetic resonance imaging (MRI), computed tomography (CT), or other real-time or pre-recorded images of the target tissue, with the real-time images preferably showing the tool system.

In another aspect, the invention provides a system for presenting an image to a user for diagnosing or treating a target tissue of a patient body. The system comprises a first image input configured to receive a first two-dimensional (2D) image dataset. The first 2D dataset defines a first image showing the target tissue and a tool receptacle of a tool delivery system disposed within the patient body. The first image has a first orientation relative to the first tool. A second image input is configured to receive a second 2D image dataset defining a second target image showing the target tissue and the tool delivery system, the second image having a second orientation relative to the tool receptacle. The second orientation is angularly offset from the first orientation. An output is configured to transmit hybrid 2D/three-dimensional (3D) image data to a display device so as to present a hybrid image for reference by the user. The hybrid image shows the first 2D image with the first orientation relative to a 3D model of the tool delivery system; and also shows the second 2D image having the second orientation relative to the 3D model. The first and second 2D images are positionally offset from the model.

In another aspect, the invention provides a method for moving a tool of a tool delivery system in a patient body with reference to a display image shown on a display. The display image shows a target tissue and the tool and defines a display coordinate system, the tool delivery system including an articulated elongate body coupled with the tool and having 1 or more, often 2 or more, and typically having 3 or more degrees of freedom. The method comprises determining, in response to a movement command entered by a hand of a user relative to the display image, a desired movement of the tool. In response to the movement command, an articulation of the elongate body is calculated so as to move the tool within the patient body, wherein the calculation of the articulation is performed by constraining the tool relative to a first plane of the display coordinate system so that the image of the tool moves along the first plane or normal to the first plane. The calculated articulation is transmitted so as to effect movement of the tool.

Optionally, a first two-dimensional (2D) image dataset is received, the first 2D dataset defining a first image showing the target tissue and the tool, the first image being along the first plane. Image data corresponding to the first 2D image dataset can be transmitted to the display device so as to generate the display image. Preferably, the display coordinate frame includes a view plane extending along a surface of the display, and the first plane will often be angularly offset from the view plane. The first plane can optionally be identified in response to a plane command from the user. The first image plane may have a first orientation relative to the tool, and a second 2D image dataset defining a second target image showing the target tissue and the tool delivery system may also be received, the second image having a second orientation relative to the receptacle. The second orientation may be angularly offset from the first orientation. The image data may be transmitted to the display, the image data comprising hybrid 2D/three-dimensional (3D) image data and the display presenting a hybrid image for reference by the user. The hybrid image may show the first 2D image with the first orientation relative to a 3D model of the tool delivery system, and the second 2D image having the second orientation relative to the 3D model. The first and second 2D images can be positionally offset from the model.

Preferably, the movement command is sensed in 1 or more, typically 2 or more, often 3 or more degrees of freedom, optionally in 5 or 6 degrees of freedom. The calculated movement command in a first mode may induce translation of the tool along the plane first plane and rotation of the tool about an axis normal to the first plane. Ideally, the calculated movement command in a second mode may induce translation of the tool normal to the first plane and rotation of the tool about an axis parallel to the first plane and normal to an axis of the tool (or along an alternative axis).

Optionally, the tool system comprises a phantom and the display image comprises an augmented reality image with a phantom image and another image of the tool. The movement command in a third mode may induce movement of the receptacle along a trajectory between the phantom image and the other image. When a workspace boundary is disposed between a location of the tool (before the commanded movement) and a desired location of the tool (as defined by the commanded movement), the movement may be limited by generating a plurality of test solutions for test movement commands at test poses of the tool along the plane. A plurality of command gradients may be determined from the candidate commands, and the movement command may be generated from the test poses and command gradients so that the commanded movement induces movement of the tool along the plane and within the workspace to adjacent the boundary.

In another aspect, the invention provides a system for moving a tool of a tool delivery system in a patient body with reference to a display image shown on a display. The display image may show a target tissue and a tool receptacle, and may define a display coordinate system. The tool delivery system may include an articulated elongate body coupled with the tool and having 3 or more degrees of freedom. The system comprises aa first processor module configured to determine, in response to a movement command entered by a hand of a user relative to the display image, a desired movement of the tool. A second processor module can be configured to determine, in response to the movement command, an articulation of the elongate body so as to move the tool within the patient body. The calculation of the articulation can be performed by constraining the tool relative to a first plane of the display coordinate system so that the image of the tool moves along the first plane, or normal to the first plane. An output can be configured to transmit the calculated articulation so as to effect movement of the tool.

Many of the system and methods described herein may be used to articulate therapeutic delivery systems and other elongate bodies having a plurality of degrees of freedom. It will often be desirable to limit the calculated articulation commands (typically generated by the processor of the system in response to input commands from the user) so that the tool, tip and/or receptacle is constrained to movement along a spatial construct, such as a plane, line, or the like. A workspace boundary will often be disposed between a current position of the receptacle and a desired position of the receptacle (as defined by the movement command from the user). Advantageously, the calculated articulation can be determined so as to induce movement of the receptacle along the spatial construct to adjacent the boundary. The constrained movement may be selected from the group consisting of translation movement in 3D space without rotation, movement along a plane, movement along a line, gimbal rotation about a plurality of intersecting axes, and rotation about an axis.

In yet another aspect, the invention provides a system for moving a tool of a tool delivery system in a patient body. The system includes an articulated elongate body coupled with the tool, the articulated tool having a boundary. The system comprises an input module configured to determine a desired spatial construct and, in response to a movement command entered by a hand of a user, a desired movement of the tool. A simulation module is coupled to the input module and is configured to determine, in response to the movement command, a plurality of alternative offset command poses of the elongate body. An articulation command module is coupled to the simulation module and configured, in response to the candidate command poses, to determine a plurality of candidate articulation commands along the construct to the simulation module; to determine a plurality of command gradients between the candidate articulation commands; and to determine an articulation command along the construct adjacent to the boundary using the gradients. The articulation command module has an output configured to transmit the articulation command so as to effect movement of the tool.

In yet another aspect, the invention provides a system for aligning a therapeutic or diagnostic tool with a target tissue adjacent an internal site in a patient. The system comprises an elongate body having a proximal end and a distal end with an axis therebetween. The body may have a receptacle configured to support the tool within the internal surgical site so that the elongate body defines a first pose. A plurality of actuators may be driving couplable with the elongate body so as to move the elongate body within the surgical site. A display may be configured to present an image including the elongate body to a user; and a processor may be couplable with the actuators and the display, the processor having a first module and a second module. The first module can be configured to receive input from the user for moving a virtual image of the elongate body from the first pose to a second pose on the display. The second module can be configured to receive a movement command, and in response, to drive the actuators so as to move the elongate body along a trajectory between the first pose and the second pose.

Preferably, an image capture system is coupled to the display and the processor. The first module can be configured to move the virtual image of the elongate body relative to a stored image of the internal surgical site. The second module can be configured to transmit image capture commands to the image capture system in response to the movement command such that the image capture system selectively images the elongate body just before the move, between the first and second pose, and/or when the move is complete, and ideally all three. The virtual image can be superimposed on the display of the elongate body, and the image processing system may be configured to intermittently image the elongate body while between the poses. Advantageously, the processor may include an image processing module configured to track the movement of the elongate body using intermittent images and the virtual image, such as images separated by more than 1/15^{th} of a second, by more than 1/10^{th} of a second or even by more than ½ second. nonetheless, the availability of the virtual image can facilitate image-guided movement with or without image processing-based position feedback, often with much less radiation to the patient and medical personnel than would be the case with standard fluoroscopy. Optionally, where the anatomy may move after a movement is planned and before the movement is completed, the processor can be configured to verify that the image data is within a desired safety threshold of expected image parameters, and if it does not, to stop the planned trajectory of the elongate body and/or alert the user that something has changed, thereby providing an automated safety mechanism.

In yet another aspect, the invention provides a system for aligning a therapeutic or diagnostic tool with a target tissue adjacent an internal site in a patient. The system comprises an elongate body having a proximal end and a distal end with an axis therebetween. The body has a receptacle configured to support the tool within the internal surgical site so that the elongate body defines a pose. A plurality of actuators can be driving couplable with the elongate body so as to move the elongate body within the surgical site. A first image capture device and a second image capture device may be included for generating first image data and second image data, respectively. A display will often be coupled to the first and second image capture devices and configured to present first and second images including the elongate body to a user, the first and second images generated using the first and second image data, respectively. A processor may be couplable with the actuators and the display, the processor having a first registration module and a second registration module. The first module can be configured for aligning a virtual image of the elongate body with the first image of the elongate body. The second module can be configured for aligning the second image of the elongate body with the virtual image.

In another aspect, the invention provides a method for driving a robotic catheter within an internal worksite of a patient body, the catheter having a passively flexible proximal portion supporting an actively articulated distal portion. The method comprises manipulating, typically manually and from outside the patient body, a proximal end of the catheter so as to induce rotational and/or axial movement of an interface between the flexible proximal portion and the distal portion, typically while the interface is within the patient body. The articulated distal portion of the catheter is articulated so as to compensate for the movement of the interface such that displacement of a distal tip of the catheter within the patient in response to the movement of the interface is inhibited.

In another optional aspect, the articulated distal portion can include a proximal articulated segment having a drive-alterable proximal curvature and a distal articulated segment having a distal drive-alterable curvature with a segment interface therebetween. The manipulating of the proximal end of the catheter may include manually rotating the proximal end of the catheter about an axis of the catheter adjacent the proximal end with a hand of a user. The rotation of the catheter may optionally be sensed, and the articulating of the articulated distal portion can be performed so as to induce precessing of the proximal curvature about the axis of the catheter adjacent the interface, optionally along with precessing of the distal curvature about the axis of the catheter adjacent the segment interface such that lateral displacement of the distal tip of the catheter in response to the manual rotation of the catheter is inhibited. Manual rotation from outside the body with a fixed catheter tip inside the body can be particularly helpful for rotation of a tool supported adjacent the tip into a desired orientation about the axis of the catheter relative to a target tissue. Relatedly, the articulated distal portion can include a proximal articulated segment having a proximal curvature and a distal articulated segment having a distal curvature with a segment interface therebetween and the manipulating of the proximal end of the catheter can comprise manually displacing the proximal end of the catheter along an axis of the catheter adjacent the proximal end with a hand of a user. The manual axial displacement of the catheter can be sensed and the articulating of the articulated distal portion can be performed so as to induce a first change in the proximal curvature and a second change in the distal curvature such that axial displacement of the distal tip of the catheter in response to the manual displacement of the catheter is inhibited, which can be useful for positioning a workspace of a tool adjacent the distal tip of the catheter so as to encompass a target tissue. The axial and/or rotational manual manipulation of the catheter outside the patient can be combined or used while driving a position of the tip to a new position relative to adjacent tissue.

In another aspect, the invention provides a system for driving a robotic catheter within an internal worksite of a patient body. The catheter can have a passively flexible proximal portion supporting an actively articulated distal portion. The system comprises a processor having a drive module configured to, in response to manipulating a proximal end of the catheter from outside the patient body so as to induce rotational and/or axial movement of an interface between the flexible proximal portion and the distal portion, transmit signals to articulate the articulated distal portion of the catheter. These drive signals can help compensate for the movement of the interface. More specifically, the drive signals can drive the tip such that displacement of a distal tip of the catheter within the patient (in response to the movement of the interface) is inhibited.

In another aspect, the invention provides a method for driving a medical robotic system. The system can be configured for manipulating a tool receptacle in a workspace within a patient body with reference to a display. The receptacle can define a first pose in the workspace and the display can show a workspace image of the receptacle and/or a tool supported thereby in the workspace. The method comprises receiving input, with a processor and relative to the workspace image, defining an input trajectory from the first pose to a desired pose of the receptacle and/or tool within the workspace. The processor can calculate a candidate trajectory from the first pose to the desired pose; and can transmit drive commands from the processor in response to the candidate trajectory so as to induce movement of the tool and/or receptacle toward the desired pose.

Optionally, the workspace image can include a tissue image of tissue adjacent the workspace. The tool and/or receptacle can be supported by an elongate flexible catheter having an image shown on the display. On the display phantom catheter with the desired pose can be superimposed, and a trajectory validation catheter between the initial pose and the desired pose. These can facilitate visual validation of catheter movement safety prior to transmitting of the drive commands. Other options include identifying a plurality of verification locations along the candidate trajectory. For any of the verification locations outside a workspace of the catheter, alternative verification locations within the workspace can be identified and a path can be smoothed in response to the verification locations and any alternative verification locations. Superimposing the validation catheter can be performed by advancing the validation catheter between the verification locations and any alternative verification locations. Still further options include identifying he first location in response to receipt, by the processor, of a command to go back to a prior pose of the catheter. The desired pose may, for example, comprise the prior pose and the catheter may have moved from the prior pose along a previous trajectory,

In yet another aspect, the invention provides a processor for driving a medical robotic system. The system can have a display and a tool receptacle movable in a workspace within a patient body with reference to the display. The receptacle can, in use, define a first pose in the workspace and the display can show a workspace image of the receptacle (and/or a tool supported thereby) in the workspace. The processor can comprise an input module configured for receiving input, relative to the workspace image, defining an input trajectory from the first pose to a desired pose of the receptacle and/or tool within the workspace. A simulation module can be configured for calculating, with the processor, a candidate trajectory from the first pose to the desired pose. An output of the processor can be configured for transmitting drive commands in response to the candidate trajectory so as to induce movement of the tool and/or receptacle toward the desired pose.

### BRIEF DESCRIPTION OF THE DRAWINGS

FIG. 1 illustrates an interventional cardiologist performing a structural heart procedure with a robotic catheter system having a fluidic catheter driver slidably supported by a stand.

FIG. 2 is a simplified schematic illustration of components of a helical balloon assembly, showing how an extruded multi-lumen shaft can provide fluid to laterally aligned subsets of balloons within an articulation balloon array of a catheter.

FIGS. 3A-3C schematically illustrate helical balloon assemblies supported by flat springs and embedded in an elastomeric polymer matrix, and also show how selective inflation of subsets of the balloons can elongate and laterally articulate the assemblies.

FIG. 4 is a perspective view of a robotic catheter system in which a catheter is removably mounted on a driver assembly, and in which the driver assembly includes a driver encased in a sterile housing and supported by a stand.

FIG. 5 schematically illustrates a robotic catheter system and transmission of signals between the components thereof so that input from a user induces a desired articulation.

FIG. 6 is a high-level flow chart of an exemplary control system for the fluid-driven robotic catheter control systems described herein.

FIG. 7 is a flow chart showing an exemplary method and structure for use by the control systems described herein to solve for the inverse kinematics of the fluid-driven catheter structures.

FIG. 8 illustrates a relationship between reference frames at the base of the articulated segments and at the tip of the catheter, for use in the control systems described herein.

FIG. 9 illustrates Euler angles for determination of transformations between reference frames used in the control systems described herein.

FIGS. 10A and 10B graphically illustrate angles and values used by the control systems described herein.

FIG. 11 is a flow chart showing inverse kinematics for a segment as can be used to solve for lumen pressures in a control system of the fluid-driven catheter systems described herein.

FIG. 12 graphically illustrate angles and values used in the user interface of the control systems described herein.

FIGS. 13A and 13B graphically illustrate angles and values used for communication of signals between the user interface and the robotic position control of the control systems described herein.

FIG. 14 schematically illustrate exemplary components of an input-output system for use in the robotic control and/or simulation systems described herein, and also show an image of a tool supported by a flexible body within an internal surgical site bordered by adjacent tissue.

FIGS. 15A and 15B schematically illustrate exemplary components of robotic control and/or simulation systems and show communications between those components.

FIGS. 16A-16D are screen prints of display images showing an in situ movement plan (or simulation thereof) and associated movement of a catheter-supported tool along a trajectory determined by disregarding one or more candidate intermediate input poses and a trajectory of a virtual catheter indicator.

FIGS. 17A-17D are screen prints of display images showing rotational and translational movements and associated indicia for use with a planar input device during movement of an actual or simulated robotic catheter.

FIGS. 18A-18C illustrate exemplary graphical indicia superimposed on an image of an actual or simulated robotic catheter to facilitate precise and predictable rotation, translation, and alignment with target tissues.

FIG. 19 is a functional block diagram of an exemplary fluid-driven structural heart therapy system having an augmented reality hybrid 2D/3D display for reference by a system user to position a therapeutic or diagnostic tool in an open chamber of a patient's beating heart.

FIGS. 20A and 20B are screen shots of an augmented reality display for use in the system of FIG. 19, showing a captured 2D image representing an actual tip of an articulated delivery system and adjacent tissue in FIG. 20A, and showing a 2D image of a virtual model of the articulated delivery system superimposed on the captured 2D tissue/tip image.

FIG. 21 is a screenshot showing a hybrid 2D/3D display for use in the system of FIG. 19, with the display presenting an image including a 3D virtual model of the articulated delivery system, and also presenting first and second 2D image planes, each having a 2D image of the virtual model projected thereon with the orientations of the 2D images relative to the model corresponding to the orientations of the planes on which they are projected, wherein the 2D image planes represent fluoroscopic image planes.

FIG. 22 is a screenshot showing another hybrid 2D/3D display presenting a 3D virtual image, a 2D fluoroscopic image plane, and first and second 2D ultrasound image slice planes, wherein the ultrasound planes are offset from the model.

FIG. 23 is a screenshot showing another hybrid 2D/3D display presenting a 3D virtual image of an articulated delivery system and an ultrasound transducer, a 2D fluoroscopic image plane having the articulated delivery system and transducer projected thereon, and first and second 2D ultrasound image slice planes of the transducer, and a 3D ultrasound image volume of the transducer so as to illustrate how image data of the fluoroscopic and ultrasound system can be registered and tracked.

FIG. 24 is a screenshot showing first and second 2D ultrasound image slice planes of a transducer, along with a 3D virtual image of an articulated delivery system and 2D virtual image sliced projected from the model to the 2D ultrasound image planes.

FIG. 25 is a screenshot showing yet another hybrid 2D/3D display showing a 3D virtual model of the articulated delivery system, offset of first and second 2D ultrasound image planes along their normals, projection of a phantom articulated delivery system onto the ultrasound image planes, and a 3D trajectory between the virtual 3D model and the 3D phantom.

FIGS. 26A-26C are screenshots showing a hybrid 2D/3D display showing 3D virtual models of articulated delivery systems and 2D images of the virtual models projected on 2D images, with both the 3D and 2D images including widgets adjacent the tip of the models, the widgets correlating to a constraint on the movement of the articulated delivery system.

FIGS. 26D-26G schematically illustrate geometric terms which may optionally be used to constrain motion of articulated devices as detailed in the associated text.

FIGS. 27A-27C are screenshots showing pages of a 6 degree of freedom input device, showing app pages and buttons for controlling the articulation system in different modes using a variety of alternative constraints.

FIGS. 28A-28C illustrate manual positioning of a 6 degree of freedom input device and corresponding movement of an image of a 3D virtual catheter within a 3D virtual workspace as shown on a 2D display, and also show spring-back of the view to a starting position and orientation when a view drive input button is released.

FIGS. 29A-29D illustrate optional image elements to be included in an exemplary hybrid 2D/3D display image.

FIGS. 30A-30C illustrate manual manipulation of the catheter body from outside the patient while driving the tip so as to inhibit resulting changes in the tip position.

FIG. 31 schematically illustrates a calculated trajectory that avoids a workspace boundary, along with a virtual trajectory verification catheter that moves along the trajectory to facilitate visual verification of safety prior to implementing a move of the actual catheter.

### DETAILED DESCRIPTION OF THE INVENTION

The improved devices, systems, and methods for controlling, image guidance of, inputting commands into, and simulating movement of powered and robotic devices will find a wide variety of uses. The elongate tool-supporting structures described herein will often be flexible, typically comprising catheters suitable for insertion in a patient body. Exemplary systems will be configured for insertion into the vascular system, the systems typically including a cardiac catheter and supporting a structural heart tool for repairing or replacing a valve of the heart, occluding an ostium or passage, or the like. Other cardiac catheter systems will be configured for diagnosis and/or treatment of congenital defects of the heart, or may comprise electrophysiology catheters configured for diagnosing or inhibiting arrhythmias (optionally by ablating a pattern of tissue bordering or near a heart chamber).

Alternative applications may include use in steerable supports of image acquisition devices such as for trans-esophageal echocardiography (TEE), intra-coronary echocardiography (ICE), and other ultrasound techniques, endoscopy, and the like. The structures described herein will often find applications for diagnosing or treating the disease states of or adjacent to the cardiovascular system, the alimentary tract, the airways, the urogenital system, and/or other lumen systems of a patient body. Other medical tools making use of the articulation systems described herein may be configured for endoscopic procedures, or even for open surgical procedures, such as for supporting, moving and aligning image capture devices, other sensor systems, or energy delivery tools, for tissue retraction or support, for therapeutic tissue remodeling tools, or the like. Alternative elongate flexible bodies that include the articulation technologies described herein may find applications in industrial applications (such as for electronic device assembly or test equipment, for orienting and positioning image acquisition devices, or the like). Still further elongate articulatable devices embodying the techniques described herein may be configured for use in consumer products, for retail applications, for entertainment, or the like, and wherever it is desirable to provide simple articulated assemblies with one or more (preferably multiple) degrees of freedom without having to resort to complex rigid linkages.

Embodiments provided herein may use balloon-like structures to effect articulation of the elongate catheter or other body. The term “articulation balloon” may be used to refer to a component which expands on inflation with a fluid and is arranged so that on expansion the primary effect is to cause articulation of the elongate body. Note that this use of such a structure is contrasted with a conventional interventional balloon whose primary effect on expansion is to cause substantial radially outward expansion from the outer profile of the overall device, for example to dilate or occlude or anchor in a vessel in which the device is located. Independently, articulated medial structures described herein will often have an articulated distal portion, and an unarticulated proximal portion, which may significantly simplify initial advancement of the structure into a patient using standard catheterization techniques.

The robotic systems described herein will often include an input device, a driver, and an articulated catheter or other robotic manipulator supporting a diagnostic or therapeutic tool. The user will typically input commands into the input device, which will generate and transmit corresponding input command signals. The driver will generally provide both power for and articulation movement control over the tool. Hence, somewhat analogous to a motor driver, the driver structures described herein will receive the input command signals from the input device and will output drive signals to the tool-supporting articulated structure so as to effect robotic movement of an articulated feature of the tool (such as movement of one or more laterally deflectable segments of a catheter in multiple degrees of freedom). The drive signals may comprise fluidic commands, such as pressurized pneumatic or hydraulic flows transmitted from the driver to the tool-supporting catheter along a plurality of fluid channels. Optionally, the drive signals may comprise electromagnetic, optical, or other signals, preferably (although not necessarily) in combination with fluidic drive signals. Unlike many robotic systems, the robotic tool supporting structure will often (though not always) have a passively flexible portion between the articulated feature (typically disposed along a distal portion of a catheter or other tool manipulator) and the driver (typically coupled to a proximal end of the catheter or tool manipulator). The system will be driven while sufficient environmental forces are imposed against the tool or catheter to impose one or more bend along this passive proximal portion, the system often being configured for use with the bend(s) resiliently deflecting an axis of the catheter or other tool manipulator by 10 degrees or more, more than 20 degrees, or even more than 45 degrees.

The catheter bodies (and many of the other elongate flexible bodies that benefit from the inventions described herein) will often be described herein as having or defining an axis, such that the axis extends along the elongate length of the body. As the bodies are flexible, the local orientation of this axis may vary along the length of the body, and while the axis will often be a central axis defined at or near a center of a cross-section of the body, eccentric axes near an outer surface of the body might also be used. It should be understood, for example, that an elongate structure that extends “along an axis” may have its longest dimension extending in an orientation that has a significant axial component, but the length of that structure need not be precisely parallel to the axis. Similarly, an elongate structure that extends “primarily along the axis” and the like will generally have a length that extends along an orientation that has a greater axial component than components in other orientations orthogonal to the axis. Other orientations may be defined relative to the axis of the body, including orientations that are transvers to the axis (which will encompass orientation that generally extend across the axis, but need not be orthogonal to the axis), orientations that are lateral to the axis (which will encompass orientations that have a significant radial component relative to the axis), orientations that are circumferential relative to the axis (which will encompass orientations that extend around the axis), and the like. The orientations of surfaces may be described herein by reference to the normal of the surface extending away from the structure underlying the surface. As an example, in a simple, solid cylindrical body that has an axis that extends from a proximal end of the body to the distal end of the body, the distal-most end of the body may be described as being distally oriented, the proximal end may be described as being proximally oriented, and the curved outer surface of the cylinder between the proximal and distal ends may be described as being radially oriented. As another example, an elongate helical structure extending axially around the above cylindrical body, with the helical structure comprising a wire with a square cross section wrapped around the cylinder at a 20 degree angle, might be described herein as having two opposed axial surfaces (with one being primarily proximally oriented, one being primarily distally oriented). The outermost surface of that wire might be described as being oriented exactly radially outwardly, while the opposed inner surface of the wire might be described as being oriented radially inwardly, and so forth.

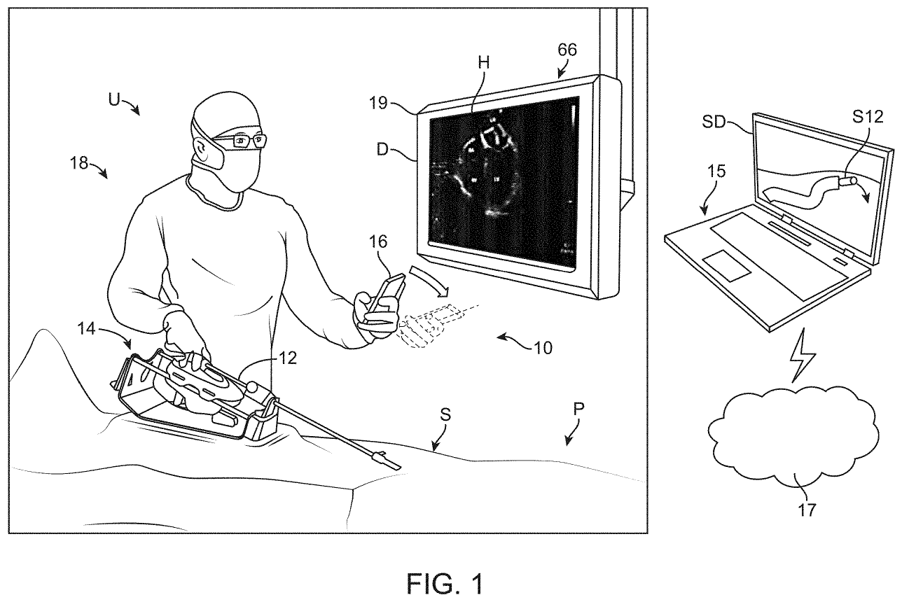

Referring first to FIG. 1, a system user U, such as an interventional cardiologist, uses a robotic catheter system 10 to perform a procedure in a heart H of a patient P. System 10 generally includes an articulated catheter 12, a driver assembly 14, and an input device 16. User U controls the position and orientation of a therapeutic or diagnostic tool mounted on a distal end of catheter 12 by entering movement commands into input 16, and optionally by sliding the catheter relative to a stand of the driver assembly, while viewing a distal end of the catheter and the surrounding tissue in a display D. As will be described below, user U may alternatively manually rotate the catheter body about its axis in some embodiments.

During use, catheter 12 extends distally from driver system 14 through a vascular access site S, optionally (though not necessarily) using an introducer sheath. A sterile field 18 encompasses access site S, catheter 12, and some or all of an outer surface of driver assembly 14. Driver assembly 14 will generally include components that power automated movement of the distal end of catheter 12 within patient P, with at least a portion of the power often being transmitted along the catheter body as a hydraulic or pneumatic fluid flow. To facilitate movement of a catheter-mounted therapeutic tool per the commands of user U, system 10 will typically include data processing circuitry, often including a processor within the driver assembly. Regarding that processor and the other data processing components of system 10, a wide variety of data processing architectures may be employed. The processor, associated pressure and/or position sensors of the driver assembly, and data input device 16, optionally together with any additional general purpose or proprietary computing device (such as a desktop PC, notebook PC, tablet, server, remote computing or interface device, or the like) will generally include a combination of data processing hardware and software, with the hardware including an input, an output (such as a sound generator, indicator lights, printer, and/or an image display), and one or more processor board(s). These components are included in a processor system capable of performing the transformations, kinematic analysis, and matrix processing functionality associated with generating the valve commands, along with the appropriate connectors, conductors, wireless telemetry, and the like. The processing capabilities may be centralized in a single processor board, or may be distributed among various components so that smaller volumes of higher-level data can be transmitted. The processor(s) will often include one or more memory or other form of volatile or non-volatile storage media, and the functionality used to perform the methods described herein will often include software or firmware embodied therein. The software will typically comprise machine-readable programming code or instructions embodied in non-volatile media and may be arranged in a wide variety of alternative code architectures, varying from a single monolithic code running on a single processor to a large number of specialized subroutines, classes, or objects being run in parallel on a number of separate processor sub-units.

Referring still to FIG. 1, along with display D, a simulation display SD may present an image of an articulated portion of a simulated or virtual catheter S12 with a receptacle for supporting a simulated therapeutic or diagnostic tool. The simulated image shown on the simulation display SD may optionally include a tissue image based on pre-treatment imaging, intra-treatment imaging, and/or a simplified virtual tissue model, or the virtual catheter may be displayed without tissue. Simulation display SD may have or be included in an associated computer 15, and the computer will preferably be couplable with a network and/or a cloud 17 so as to facilitate updating of the system, uploading of treatment and/or simulation data for use in data analytics, and the like. Computer 15 may have a wireless, wired, or optical connection with input device 16, a processor of driver assembly 14, display D, and/or cloud 17, with suitable wireless connections comprising a Bluetooth™ connection, a WiFi connection, or the like. Preferably, an orientation and other characteristics of simulated catheter S12 may be controlled by the user U via input device 16 or another input device of computer 15, and/or by software of the computer so as to present the simulated catheter to the user with an orientation corresponding to the orientation of the actual catheter as sensed by a remote imaging system (typically a fluoroscopic imaging system, an ultra-sound imaging system, a magnetic resonance imaging system (MRI), or the like) incorporating display D and an image capture device 19. Optionally, computer 15 may superimpose an image of simulated catheter S12 on the tissue image shown by display D (instead of or in addition to displaying the simulated catheter on simulation display SD), preferably with the image of the simulated catheter being registered with the image of the tissue and/or with an image of the actual catheter structure in the surgical site. Still other alternatives may be provided, including presenting a simulation window showing simulated catheter SD on display D, including the simulation data processing capabilities of computer 15 in a processor of driver assembly 14 and/or input device 16 (with the input device optionally taking the form of a tablet that can be supported by or near driver assembly 14, incorporating the input device, computer, and one or both of displays D, SD into a workstation near the patient, shielded from the imaging system, and/or remote from the patient, or the like.

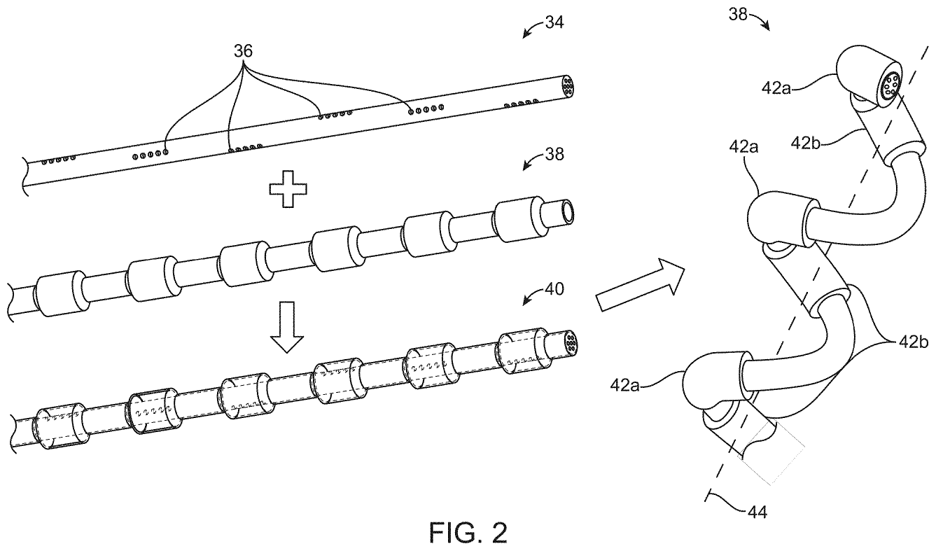

Referring now to FIG. 2, the components of, and fabrication method for production of, an exemplary balloon array assembly (sometimes referred to herein as a balloon string 32) can be understood. A multi-lumen shaft 34 will typically have between 3 and 18 lumens. The shaft can be formed by extrusion with a polymer such as a nylon, a polyurethane, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like. A series of ports 36 are formed between the outer surface of shaft 36 and the lumens, and a continuous balloon tube 38 is slid over the shaft and ports, with the ports being disposed in large profile regions of the tube and the tube being sealed over the shaft along the small profile regions of the tube between ports to form a series of balloons. The balloon tube may be formed using a compliant, non-compliant, or semi-compliant balloon material such as a latex, a silicone, a nylon elastomer, a polyurethane, a nylon, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like, with the large-profile regions preferably being blown sequentially or simultaneously to provide desired hoop strength. The ports can be formed by laser drilling or mechanical skiving of the multi-lumen shaft with a mandrel in the lumens. Each lumen of the shaft may be associated with between 3 and 50 balloons, typically from about 5 to about 30 balloons. The shaft balloon assembly 40 can be coiled to a helical balloon array of balloon string 32, with one subset of balloons 42a being aligned along one side of the helical axis 44, another subset of balloons 44b (typically offset from the first set by 120 degrees) aligned along another side, and a third set (shown schematically as deflated) along a third side. Alternative embodiments may have four subsets of balloons arranged in quadrature about axis 44, with 90 degrees between adjacent sets of balloons.

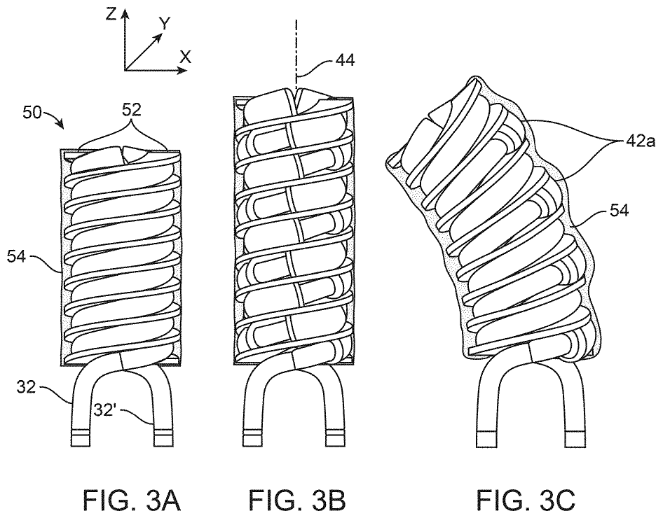

Referring now to FIGS. 3A, 3B, and 3C, an articulated segment assembly 50 has a plurality of helical balloon strings 32, 32′ arranged in a double helix configuration. A pair of flat springs 52 are interleaved between the balloon strings and can help axially compress the assembly and urge deflation of the balloons. As can be understood by a comparison of FIGS. 3A and 3B, inflation of subsets of the balloons surrounding the axis of segment 50 can induce axial elongation of the segment. As can be understood with reference to FIGS. 3A and 3C, selective inflation of a balloon subset 42a offset from the segment axis 44 along a common lateral bending orientation X induces lateral bending of the axis 44 away from the inflated balloons. Variable inflation of three or four subsets of balloons (via three or four channels of a single multi-lumen shaft, for example) can provide control over the articulation of segment 50 in three degrees of freedom, i.e., lateral bending in the +/−X orientation and the +/−Y orientation, and elongation in the +Z orientation. As noted above, each multilumen shaft of the balloon strings 32, 32′ may have more than three channels (with the exemplary shafts having 6 or 7 lumens), so that the total balloon array may include a series of independently articulatable segments (each having 3 or 4 dedicated lumens of one of the multi-lumen shafts, for example). Optionally, from 2 to 4 modular, axially sequential segments may each have an associated tri-lumen shaft, with the tri-lumen shaft extending axially in a loose helical coil through the lumen of any proximal segments to accommodate bending and elongation. The segments may each include a single helical balloon string/multilumen shaft assembly (rather than having a dual-helix configuration). Multi-lumen shafts for driving of distal segments may alternatively wind proximally around an outer surface of a proximal segment, or may be wound parallel and next to the multi-lumen shaft/balloon tube assemblies of the balloon array of the proximal segment(s).

Referring still to FIGS. 3A, 3B, and 3C, articulated segment 50 optionally includes a polymer matrix 54, with some or all of the outer surface of balloon strings 32, 32′ and flat springs 52 that are included in the segment being covered by the matrix. Matrix 54 may comprise, for example, a relatively soft elastomer to accommodate inflation of the balloons and associated articulation of the segment, with the matrix optionally helping to urge the balloons toward an at least nominally deflated state, and to urge the segment toward a straight, minimal length configuration. Alternatively (or in addition to a relatively soft matrix), a thin layer of relatively high-strength elastomer can be applied to the assembly (prior to, after, or instead of the soft matrix), optionally while the balloons are in an at least partially inflated state. Advantageously, matrix 54 can help maintain overall alignment of the balloon array and springs within the segment despite segment articulation and bending of the segment by environmental forces. Regardless of whether or not a matrix is included, an inner sheath may extend along the inner surface of the helical assembly, and an outer sheath may extend along an outer surface of the assembly, with the inner and/or outer sheaths optionally comprising a polymer reinforced with wire or a high-strength fiber in a coiled, braid, or other circumferential configuration to provide hoop strength while accommodating lateral bending (and preferably axial elongation as well). The inner and outer sheaths may be sealed together distal of the balloon assembly, forming an annular chamber with the balloon array disposed therein. A passage may extend proximally from the annular space around the balloons to the proximal end of the catheter to safely vent any escaping inflation media, or a vacuum may be drawn in the annular space and monitored electronically with a pressure sensor to inhibit inflation flow if the vacuum deteriorates.

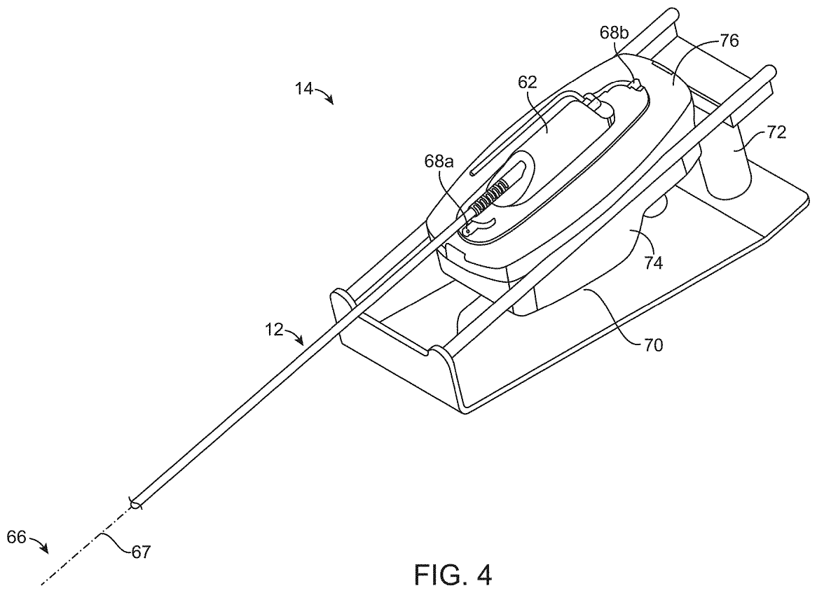

Referring now to FIG. 4, a proximal housing 62 of catheter 12 and the primary components of driver assembly 14 can be seen in more detail. Catheter 12 generally includes a catheter body 64 that extends from proximal housing 62 to an articulated distal portion 66 (see FIG. 1) along an axis 67, with the articulated distal portion preferably comprising a balloon array and the associated structures described above. Proximal housing 62 also contains first and second rotating latch receptacles 68a, 68b which allow a quick-disconnect removal and replacement of the catheter. The components of driver assembly 14 visible in FIG. 4 include a sterile housing 70 and a stand 72, with the stand supporting the sterile housing so that the sterile housing (and components of the driver assembly therein, including the driver) and catheter 12 can move axially along axis 67. Sterile housing 70 generally includes a lower housing 74 and a sterile junction having a sterile barrier 76. Sterile junction 76 releasably latches to lower housing 74 and includes a sterile barrier body that extends between catheter 12 and the driver contained within the sterile housing. Along with components that allow articulation fluid flow to pass through the sterile fluidic junction, the sterile barrier may also include one or more electrical connectors or contacts to facilitate data and/or electrical power transmission between the catheter and driver, such as for articulation feedback sensing, manual articulations sensing, or the like. The sterile housing 70 will often comprise a polymer such as an ABS plastic, a polycarbonate, acetal, polystyrene, polypropylene, or the like, and may be injection molded, blow molded, thermoformed, 3-D printed, or formed using still other techniques. Polymer sterile housings may be disposable after use on a single patient, may be sterilizable for use with a limited number of patients, or may be sterilizable indefinitely; alternative sterile housings may comprise metal for long-term repeated sterile processing. Stand 72 will often comprise a metal, such as a stainless steel, aluminum, or the like for repeated sterilizing and use.

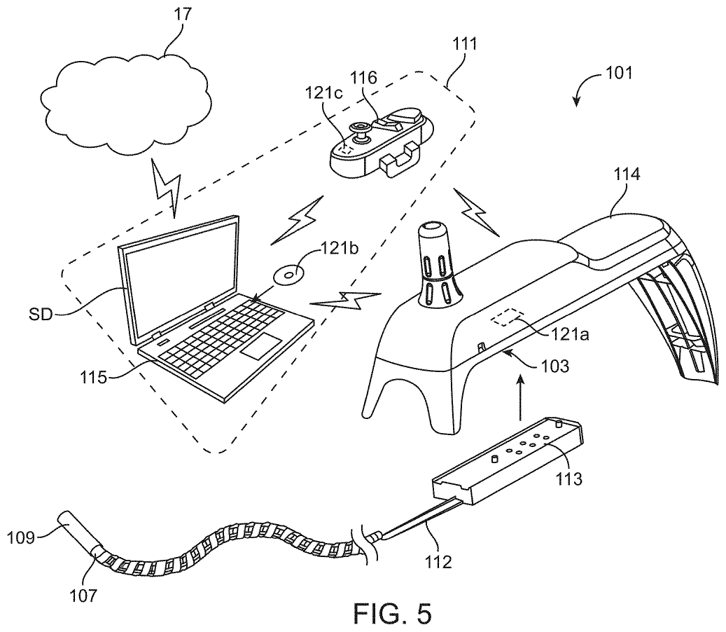

Referring now to FIG. 5, components of a simulation system 101 that can be used for simulation, training, pre-treatment planning, and or treatment of a patent are schematically illustrated. Some or all of the components of system 101 may be used in addition to or instead of the clinical components of the system shown in FIG. 1. System 101 may optionally include an alternative catheter 112 and an alternative driver assembly 114, with the alternative catheter comprising a real and/or virtual catheter and the driver assembly comprising a real and/or virtual driver 114.

Alternative catheter 112 can be replaceably coupled with alternative driver assembly 114. When simulation system 101 is used for driving an actual catheter, the coupling may be performed using a quick-release engagement between an interface 113 on a proximal housing of the catheter and a catheter receptacle 103 of the driver assembly. An elongate body 105 of catheter 112 has a proximal/distal axis as described above and a distal receptacle 107 that is configured to support a therapeutic or diagnostic tool 109 such as a structural heart tool for repairing or replacing a valve of a heart. The tool receptacle may comprise an axial lumen for receiving the tool within or through the catheter body, a surface of the body to which the tool is permanently affixed, or the like. Alternative drive assembly 114 may be wireless coupled to a simulation computer 115 and/or a simulation input device 116, or cables may be used for transmission of data.

When alternative catheter 112 and alternative drive system 114 comprise virtual structures, they may be embodied as modules of software, firmware, and/or hardware. The modules may optionally be configured for performing articulation calculations modeling performance of some or all of the actual clinical components as described below, and/or may be embodied as a series of look-up tables to allow computer 115 to generate a display effectively representing the performance. The modules will optionally be embodied at least in-part in a non-volatile memory of a simulation-supporting alternative drive assembly 121a, but some or all of the simulation modules will preferably be embodied as software in non-volatile memories 121b, 121c of simulation computer 115 and/or simulation input device 116, respectively. Coupling of alternative virtual catheters and tools can be permed using menu options or the like. In some embodiments, selection of a virtual catheter may be facilitated by a signal generated in response to mounting of an actual catheter to an actual driver.

Simulation computer 115 preferably comprises an off-the-shelf notebook or desktop computer that can be coupled to cloud 17, optionally via an intranet, the internet, an ethernet, or the like, typically using a wireless router or a cable coupling the simulation computer to a server. Cloud 17 will preferably provide data communication between simulation computer 115 and a remote server, with the remote server also being in communication with a processor of other simulation computers 115 and/or one or more clinical drive assemblies 14. Simulation computer 115 may also comprise code with a virtual 3D workspace, the workspace optionally being generated using a proprietary or commercially available 3D development engine that can also be used for developing games and the like, such as Unity™ as commercialized by Unity Technologies. Suitable off-the-shelf computers may include any of a variety of operating systems (such as Windows from Microsoft, OS from Apple, Linex, or the like), along with a variety of additional proprietary and commercially available apps and programs.

Simulation input device 116 may comprise an off-the-shelf input device having a sensor system for measuring input commands in at least two degrees of freedom, preferably in 3 or more degrees of freedom, and in some cases 5, 6, or more degrees of freedom. Suitable off-the-shelf input devices include a mouse (optionally with a scroll wheel or the like to facilitate input in a 3^{rd} degree of freedom), a tablet or phone having an X-Y touch screen (optionally with AR capabilities such as being compliant with ARCor from Google, ARKit from Apple, or the like to facilitate input of translation and/or rotation, along with multi-finger gestures such as pinching, rotation, and the like), a gamepad, a 3D mouse, a 3D stylus, or the like. Proprietary code may be loaded on the simulation input device (particularly when a phone, tablet, or other device having a touchscreen is used), with such input device code presenting menu options for inputting additional commands and changing modes of operation of the simulation or clinical system. A simulation input/output system 111 may be defined by the simulation input device 116 and the simulation display SD.

System Motion Equations

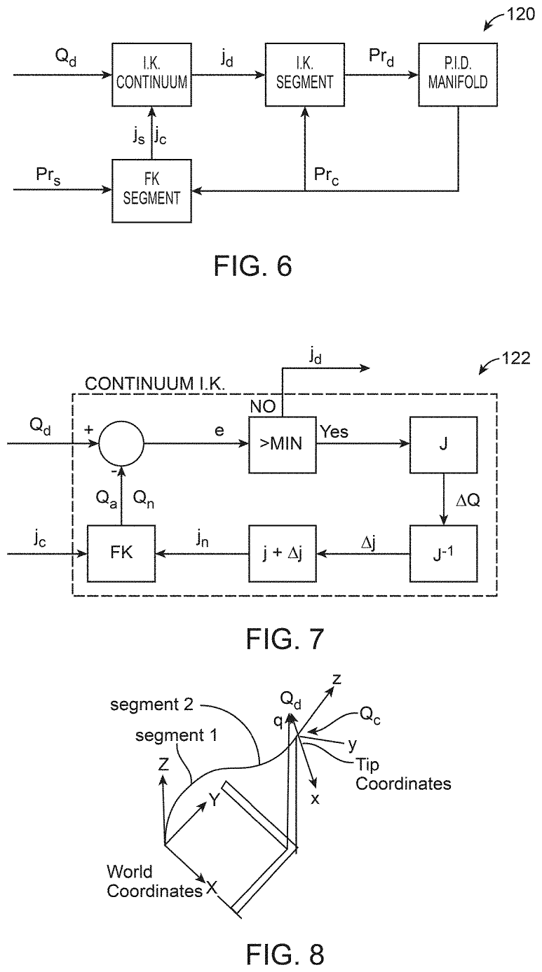

Referring now to FIG. 6, a system control flow chart 120 is shown that can be used by a processor of the robotic system to drive movement of an actual or virtual catheter. The following terms are used in flow chart 120 and/or in the analysis that follows. The following notation may be used in at least some of the equations herein:

Input

| | |
|---|---|
| Q_{d} | Tip position desired |

Measured

| | |
|---|---|
| Pr₀ | Pressure for balloon arrays |

Calculated

| | |
|---|---|
| Pr_{d} | Pressure for balloon arrays as computed for joint, j_{d} |
| j | Joint space variables |
| j_{d} | Joint space computed for desired tip position, Q_{d} |
| jₙ | Joint space newly computed |
| + | Add increment to each variable one at a time |
| Q | Tip position in world space |
| Q+ | Tip positions by added increments to each variable one at a time |
| | to form Jacobian |
| Qₙ | Tip position newly calculated |
| e | Error |
| Seg FK | Segment Forward Kinematics; pressures to joint space |
| Seg IK | Segment Inverse Kinematics; joint space to pressures |
| FK | System Forward Kinematics; joint space to world space (distal |
| | segments tip position) |
| IK | System Inverse Kinematics; world space (distal segment tip |
| | position) to joint space |
| J | Jacobian (numerically derived) |
| J⁻¹ | Inverse Jacobian |

Referring now to FIG. 7, an inverse kinematics flow chart 122 is useful to understand how the processor can solve for joint angles and displacements. The calculations below reference the variables outlined in Table I. The input variables (α0, α1, α2, α3, α4, β1, β, β3, β4, S0, S1, S2, S3, and S4) are used as input in these calculations. The calculations are arranged for articulated catheters having up to 4 articulated independently articulatable segments (segment Nos. 1-4), and a base segment (No. 0) can be used to accommodate a pose of the catheter body proximally of the most proximal articulated segment (the parameters for the base segment or segment No. 0 being treated as defined parameters in the calculations).

TABLE I
Variable Matrix

| Segment no. | n | i | 0 | 1 | 2 | 3 | 4 | |
|---|---|---|---|---|---|---|---|---|
| Angles | Alpha | αᵢ | α₀ | α₁ | α₂ | α₃ | α₄ | Input |
| | Beta | βᵢ | | β₁ | β₂ | β₃ | β₄ | |
| Arc Length | Arc | Sᵢ | S₀ | S₁ | S₂ | S₃ | S₄ | |
| Arc Radii | Radius | rᵢ | | r₁ | r₂ | r₃ | r₄ | Solve |
| Arc | Pᵢ | xᵢ | 0 | x₁ | x₂ | x₃ | x₄ | For |
| End | | yᵢ | 0 | y₁ | y₂ | Y₃ | y₄ | |
| Points | | zᵢ | z₀ | z₁ | z₂ | z₃ | z₄ | |
| Arc | Vᵢ | aᵢ | 0 | a₁ | a₂ | a₃ | a₄ | |
| Unit | | bᵢ | 0 | b₁ | b₂ | b₃ | b₄ | |
| Vectors | | cᵢ | 1 | c₁ | c₂ | c₃ | c₄ | |

Startup Position:

Segments start by expanding the balloon array inflations to predetermined levels. The Segment is driven to predetermined and straight (or nearly straight) condition defined by an initial joint space vector jₛ, which accounts for all the Segments' initial states.

To move to a desired location, balloon array conditions are changed to locate segment angles and displacement. The first step is to determine the current and desired position vectors for the robot tip in the robot's base coordinate system, sometime referred to herein as world space. Note that world space may move relative to the patient and/or the operating room with manual repositioning of the catheter or the like, so that this world space may differ from a fixed room space or global world space.

User Input Commands/Tip Vector q:

Referring now to FIG. 8, The robot is driven by a user input vector q measured from a coordinate system attached to the distal end of the robot, or tip. This q vector is transformed into a desired world space vector Q_{d}, where the current tip location in world space vector is Q_{c}.

Q_{C}=(X_{C},Y_{C},Z_{C}, [symbol] _{c}, [symbol] _{c})

The user input q represents a velocity (or small displacements) command. The tip coordinate system resides at the current tip position, and therefore the current q, or q_{c} is always at the origin:

q_{c}=(0,0,0,0,0)

Since q_{c} is zero the desired displacement, q_{d}, is equivalent to the change in q or dq as shown here:

dq=q_{d}−q_{c}=q_{d}

To simplify, dq is replaced simply with q to represent the desired change in position,

q=q_{d}=(x_{T},y_{T},z_{T},α_{T},β_{T}),

where x_{T}, y_{T}, z_{T}, α_{T}, and β_{T} describes a change vector in tip space coordinates. q is then used by the current Transformation Matrix, T_{0Tc}, to acquire the desired world coordinates, Q_{d}.

Q_{d}=T_{0Tc}(q_{d})=(X_{d},Y_{d},Z_{d}, [symbol] _{d}, [symbol] _{d})

Where the Tip's current world coordinates are defined by Q_{c}.

Q_{C}=T_{0Tc}(q_{c})=(X_{C},Y_{C},Z_{C}, [symbol] _{c}, [symbol] _{c})

Use Joint Space vector, J, contains the Segment angles and displacement information which is used to solve for the Transformation and Rotation matrix, T_{0T} and R_{0T} respectively. The Transformation and Rotation Matrix will be discussed below.

Current Catheter State or Pose

### Q_{C}:

The current world coordinate vector Q_{C} is defined by the tip q vector with no displacement which is q_{c}, and can be resolved as follows:

Q_{C}=T_{0Tc}(q_{c})=(X_{C},Y_{C},Z_{C}, [symbol] _{c}, [symbol] _{c})

The coordinates may be found by the following math,

(X_{C},Y_{C},Z_{C},1)^{T}=T_{0Tc}·(0,0,0,1)^{T}

a_{T}=cos(0)*sin(0)=0

b_{T}=sin(0)*sin(0)=0

c_{T}=cos(0)=1

(A_{c},B_{c},C_{c},0)^{T}=T_{0Tc}·(0,0,1,0)^{T}

[symbol]

_{c}=atan 2(A_{c},B_{c})

[symbol]

_{c}=atan 2(C_{c},H_{c})

H_{c}=(A_{c}²+B_{c}²)^{1/2}

The range of Beta ( [symbol] ) may be limited if the rotation matrix is not used. This is due to use of the hypotenuse (H) quantity which removes the negative sign from one side of the atan 2 formula as follows:

H=(A²B²)^{1/2}

[symbol]

=atan 2(C,H), 0< [symbol] <180

### Q_{d}.

The desired world coordinate vector Q_{d} is defined by the tip q vector with desired displacement which is q_{d}, and can be resolved as follows:

Q_{d}=T_{0Tc}(q_{d})=(X_{d},Y_{d},Z_{d}, [symbol] _{d}, [symbol] _{d})

The coordinates may be found by the following math,

(X_{d},Y_{d},Z_{d},1)^{T}=T_{0T}·(x_{T},y_{T},z_{T},1)^{T}

a_{T}=cos(α_{T})*sin(β_{T}); b_{T}=sin(α_{T})*sin(β_{T}); c_{T}=cos(β_{T})

(A_{d},B_{d},C_{d},0)^{T}=T_{0T}·(a_{T},b_{T},c_{T},0)^{T}

[symbol]

_{d}=atan 2(A_{d},B_{d})

[symbol]

_{d}=atan 2(C_{d},H_{d})

H_{d}=(A_{d}²+B_{d}²)^{1/2}

The following alternative formula solves Beta (B) in all four quadrants for a full 360 degrees (as opposed to only two quadrants and 180 degrees) and for Gamma (F), the sixth and final coordinate to define the position in 3D space.

### Q_{C}:

Use J_{c} (current Joint Space variables) is used to solve for the current T_{0Tc} and R_{0Tc}

(X_{C},Y_{C},Z_{C},1)^{T}=T_{0Tc}·(0,0,0,1)^{T}

(a_{Tx},b_{Tx},c_{Tx})^{T}=R_{0Tc}×(1,0,0)^{T}

(a_{Ty},b_{Ty},c_{Ty})^{T}=R_{0Tc}×(0,1,0)^{T}

(a_{Tz},b_{Tz},c_{Tz})^{T}=R_{0Tc}×(0,0,1)^{T}

[symbol]

_{c}=atan 2(a_{Tz},b_{Tz})

if |a_{Tz}|<min; than a_{Tz}=(a_{Tz}/|a_{Tz}|)*min

[symbol]

_{c}=atan 2(c_{αz},a_{αt})

a_{αz}=cos α*a_{Tz}+sin α*b_{Tz}

C_{αz}=C_{Tz}

if |c_{αz}|<min; than c_{αz}=(c_{αz}/|c_{αz}|)*min

Γ_{c}=atan 2(a_{βx},b_{βx})

a_{βx}=cos α*cos β*a_{Tx}+sin α*cos β*b_{Tx}−sin β*c_{Tx}

b_{βx}=−sin α*a_{Tx}+cos α*b_{Tx}

if |a_{βx}|<min; than a_{βx}=(a_{βx}/|a_{βx})*min

### Q_{d}:

(X_{d},Y_{d},Z_{d},1)^{T}=T_{0Tc}—(X_{d},Y_{d},Z_{d},1)^{T}

Use Q_{d} with Inverse Jacobian to solve for Segment angles and displacements, J_{d} (desired Joint Space variables).
Use J_{d} to solve for the new T_{0Td} and R_{0Td}

(X_{d},Y_{d},Z_{d},1)^{T}=T_{0Td}·(0,0,0,1)^{T}

(a_{Tx},b_{Tx},c_{Tx})^{T}=R_{0Td}×(1,0,0)^{T}

(a_{Ty},b_{Ty},c_{Ty})^{T}=R_{0Td}×(0,1,0)^{T}

(a_{Tz},b_{Tz},c_{Tz})^{T}=R_{0Td}×(0,0,1)^{T}

[symbol]

_{d}=atan 2(a_{Tz},b_{Tz})

if |a_{Tz}|<min; than a_{Tz}=(a_{Tz}/|a_{Tz}|)*min

[symbol]

_{d}=atan 2(c_{αz},a_{αz})

a_{αz}=cos α*a_{Tz}+sin α*b_{Tz}

c_{αz}=c_{Tz}

if |c_{αz}|<min; than c_{αz}=(c_{αz}/|c_{αz}|)*min

Γ_{d}=atan 2(a_{βx},b_{βx})

a_{βx}=cos α*cos β*a_{Tx}+sin α*cos β*b_{Tx}−sin β*c_{Tx}

b_{βx}=−sin α*a_{Tx}+cos α*b_{Tx}

if |a_{βx}|<min; than a_{βx}=(a_{βx}/|a_{βx}|)*min

Basis for Alternative Formulas:

Solve for base coordinate axis unit vectors

(a_{Tx},b_{Tx},c_{Tx})^{T}=R_{0T}×(1,0,0)^{T}

(a_{Ty},b_{Ty},c_{Ty})^{T}=R_{0T}×(0,1,0)^{T}

(a_{Tz},b_{Tz},c_{Tx})^{T}=R_{0T}×(0,0,1)^{T}

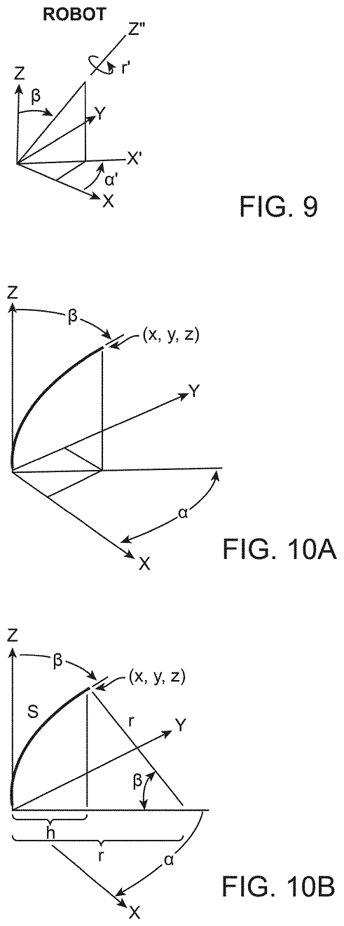

Referring now to FIG. 9, the Robot angles are resolved as follows:

α_{T}=atan 2(a_{Tz},b_{Tz})

The rotation matrix for alpha (α) is the following:

$${R}_{α} = ( \begin{matrix}cos α & - s in α & 0 \\ sin α & cos α & 0 \\ 0 & 0 & 1\end{matrix} )$$

The inverse of this rotation matrix is the transpose.

$${R}_{α}^{- 1} = {R}_{α}^{T} = ( \begin{matrix}cos α & sin α & 0 \\ - s in α & cos α & 0 \\ 0 & 0 & 1\end{matrix} ) \\ {( {a}_{α z} , {b}_{α z} , {c}_{α z} )}^{T} = {R}_{α}^{T} {x ( {a}_{T z} , {b}_{T z} , {c}_{T z} )}^{T}$$

Applying the rotation R_{α}^{T} removes the b_{α} component and aligns the beta (β) angle within X′-Z′ plane. This allows full circumferential angle determination of beta (β).

β_{T}=atan 2(c_{αz},a_{αz})

a_{αz}=cos α*a_{Tz}+sin α*b_{Tz}

b_{αz}=−sin α*a_{Tz}+cos α*b_{Tz}=0

c_{αz}=c_{Tz}

To find gamma (γ) use alpha (α) and beta (β) to create a rotation matrix as follows:

$${R}_{α β} = ( \begin{matrix}cos α & - s in α & 0 \\ sin α & cos α & 0 \\ 0 & 0 & 1\end{matrix} ) ( \begin{matrix}cos β & 0 & sin β \\ 0 & 1 & 0 \\ - s in β & 0 & cos β\end{matrix} ) \\ {R}_{α β} = ( \begin{matrix}c α * c β & - s α & c α * s β \\ s α * c β & c α & s α * s β \\ - s β & 0 & c β\end{matrix} )$$

Remove the alpha (α) and beta (β) from the Y axis vector to solve for gamma (γ). This can be done by inverting the rotation matrix and multiply by Y axis unit vector. The inverse of this rotation matrix is the transpose.

$${R}_{αβ}^{- 1} = {R}_{α β}^{T} = ( \begin{matrix}c α * c β & s α * c β & - s β \\ - s α & c α & 0 \\ c α * s β & s α * s β & c β\end{matrix} )$$

$${( {a}_{β x} , {b}_{β x} , {c}_{β x} )}^{T} = {R}_{z y}^{T} {x ( {a}_{T x} , {b}_{T x} , {c}_{T x} )}^{T}$$

This new Roll vector should have a zero in the c_{Tr} positions placing the vector on a Tip coordinate X-Y plane with values for a_{Tr} and b_{Tr}. Use these two values to determine gamma (γ) as follows:

γ_{T}=atan 2(a_{βx},b_{βx})

a_{βx}=cos β*cos β*a_{Tx}+sin α*cos β*b_{Tx}−sin β*c_{Tx}

b_{βx}=−sin α*a_{Tx}+cos α*b_{Tx}

c_{βx}=cos α*sin β*a_{Tx}+sin α*sin β*b_{Tx}+cos β*c_{Tx}=0

Limiting Input Commands to Facilitate Solution:

As discussed in the previous section, this user input vector q is used to find the desired world space vector Q_{d} using the Transformation Matrix T_{0T}. The Q_{d} vector finds the desired coordinate values for X_{d}, Y_{d}, Z_{d}, [symbol] _{d}, and [symbol] _{d}.

Due to coordinate frame limitations, beta ( [symbol] ) should be greater than zero and less than 180 degrees. As beta approaches these limits, the Inverse Kinematics solver may become unstable. This instability is remediated by assigning a maximum and minimum value for beta that is higher than zero and lower than 180 degrees. How close to the limits depends on multiple variables and it is best to validate stability with assigned limits. For example, a suitable beta minimum may be 0.01 degrees and maximum 178 degrees.

The optimization scheme used to solve for the joint vector j (through the Inverse Kinematics) may become unstable with large changes in position and angles. While large displacements and orientation changes will often resolve, there are times when it may not. Limiting the position and angles change will help maintain mathematical stability. For large q changes, dividing the path into multiple steps may be helpful, optionally with a Trajectory planner. For reference, the maximum displacement per command may be set for 3.464 mm and maximum angle set for 2 degrees. The displacement is defined by the following:

Displacement=(x_{T}²y_{T}²+z_{T}²)^{1/2}<2 mm

Angle=β_{T} degrees

Segment Rotational Matrix:

Referring now to FIG. 10A, solve rotation matrix for an arc of a cord (no axial twist).

R=Joint Rotational matrix.

$$R = {R}_{z} \times {R}_{y} \times {{R}_{z}}^{T}$$

$${R}_{z} = ( \begin{matrix}cos ( α ) & - sin ( α ) & 0 \\ sin ( α ) & cos ( α ) & 0 \\ 0 & 0 & 1\end{matrix} ) about Z axis$$

$${R}_{y} = ( \begin{matrix}cos ( β ) & 0 & sin ( β ) \\ 0 & 1 & 0 \\ - sin ( β ) & 0 & cos ( β )\end{matrix} ) about Y axis$$

$${R}_{z y} = {R}_{z} \times {R}_{y} = ( \begin{matrix}cos ( α ) & - sin ( α ) & 0 \\ sin ( α ) & cos ( α ) & 0 \\ 0 & 0 & 1\end{matrix} ) \times ( \begin{matrix}cos ( β ) & 0 & sin ( β ) \\ 0 & 1 & 0 \\ - sin ( β ) & 0 & cos ( β )\end{matrix} )$$

$$R = {R}_{z y} \times {{R}_{z}}^{T} = ( \begin{matrix}cα * c β & - s α & cα * sβ \\ sα * cβ & c α & sα * sβ \\ - sβ & 0 & cβ\end{matrix} ) \times ( \begin{matrix}c α & s α & 0 \\ - s α & c α & 0 \\ 0 & 0 & 1\end{matrix} )$$

$$R = ( \begin{matrix}c {α}^{2} * cβ + {sα}^{2} & s α * c α * ( c β - 1 ) & c α * s β \\ s α * c α * ( c β - 1 ) & s {α}^{2} * cβ + {cα}^{2} & s α * s β \\ - s β * c α & - s β * s α & c β\end{matrix} )$$

Segment Position Matrix:

Referring now to FIG. 10B, solve position matrix for the cord

$$P = ( \begin{matrix}x \\ y \\ z\end{matrix} )$$

P=point in space relative to reference frame

r=S/β

x=r*cα*[1−cβ]

x=(S/β)*cα*[1−cβ]

y=(S/β)*sα*[1−cβ]

z=(S/β)*sβ

$$P = ( \begin{matrix}( \text{S} \text{/β} ) * cα * [ 1 - cβ ] \\ ( \text{S/β} ) * sα * [ 1 - cβ ] \\ ( \text{S/β} ) * sβ\end{matrix} )$$

Segment Transformation Matrix:

Combine rotation and position matrix into a transformation matrix.

$$T = ( \begin{matrix}R & P \\ 0 & 1\end{matrix} )$$

$$T = ( \begin{matrix}\begin{matrix}{cα}^{2} * cβ + {sα}^{2} & sα * cα * ( 1 - cβ ) \\ cα * sβ & ( \text{S} \text{/β)} * cα * [ 1 - cβ ]\end{matrix} \\ \begin{matrix}sα * cα * ( 1 - cβ ) & {sα}^{2} * cβ + {cα}^{2} \\ sα * sβ & ( \text{S} \text{/β)} * sα * [ 1 - cβ ]\end{matrix} \\ \begin{matrix}\begin{matrix}cα * sβ & - sα * sβ\end{matrix} \\ \begin{matrix}cβ & ( \text{S} \text{/β)} * sβ\end{matrix}\end{matrix} \\ \begin{matrix}0 & 0 \\ 0 & 1\end{matrix}\end{matrix} )$$

Rotational Matrix Generalized:

It, indicates the rotation of a reference frame attached to the tip of segment “i” relative to a reference frame attached to its base (or the tip of Segment “i−1”).

Rᵢ=R(αᵢ,βᵢ,Sᵢ); i=0,1,2, . . . ,n;

i=0, sensor readings to input by manual (rotation & axial motion) actuation of the catheter proximal of the first segment.
i=1, is the most proximal segment.
i=n, the most distal segment (which is 2 for a two-segment catheter).

System Position Generalized:

Pᵢ indicates the origin of the distal end of segment “i” relative to a reference frame attached to its base (or the tip of Segment “i−1”).

$${P}_{i} = ( \begin{matrix}{x}_{i} \\ {y}_{i} \\ {z}_{i}\end{matrix} )$$

Continuum Translation Matrix:

Tᵢ is the transformation matrix from a frame at the distal end of segment “i” to a frame attached to its base (or the tip of Segment “i−1”).

$${T}_{i} = ( \begin{matrix}{R}_{i} & {P}_{i} \\ 0 & 1\end{matrix} ) , for i from 1 to n . \\ {T}_{i} = ( \begin{matrix}\begin{matrix}c {α}_{i}^{2} * cβi + {sα}_{i}^{2} & s {α}_{i} * c {α}_{i} * ( 1 - c {β}_{i} ) \\ c {α}_{i} * s {β}_{i} & ( {S}_{i} / {β}_{i} ) * {cα}_{i} * [ 1 - {cβ}_{i} ]\end{matrix} \\ \begin{matrix}s {α}_{i} * c {α}_{i} * ( 1 - c {β}_{i} ) & s {α}_{i}^{2} * c {β}_{i} + c {α}_{i}^{2} \\ s {α}_{i} * s {β}_{i} & ( {S}_{i} / {β}_{i} ) * {sα}_{i} * [ 1 - {cβ}_{i} ]\end{matrix} \\ \begin{matrix}- c {α}_{i} * s {β}_{i} & - s {α}_{i} * s {β}_{i} \\ c {β}_{i} & ( {S}_{i} / {β}_{i} ) * {sβ}_{i}\end{matrix} \\ \begin{matrix}0 & 0 \\ 0 & 1\end{matrix}\end{matrix} )$$

T_{w} is the transformation matrix from the most distal segment's tip reference frame to the world reference frame which is located proximal to the manually (versus fluidically) driven joints.

T_{w}=T₀T₁×T₂× . . . Tₙ

Use this matrix to solve the Forward Kinematics for current tip position Pw and axial unit vector V_{wz}.

P_{w}=T_{w}*(0,0,0,1)

P_{w}=(x_{w},y_{w},z_{w})

V_{w}=T_{w}*(0,0,1,0)

V_{wz}=(a_{wz},b_{wz},c_{wz})

Combine for World Space Tip Position:

Solve tip world space Q w, by combining Pw and V_{wz} as follows.

Qw=(Xw,Yw,Zw,αw,βw)

Xw=X_{w}=Xₙ

Yw=y_{w}=yₙ

Zw=Z_{w}=Zₙ

### Solve for Segment Angles:

α_{w}=atan 2(a_{wz},b_{wz})

if |a_{wz}|<min; than a_{wz}=(a_{wz}/|a_{wz}|)*min

### Current β_{w}

β_{w}=atan 2(c_{wz},ab_{wz})

ab_{wz}=(a_{wz}²+b_{wz}²)^{1/2}

if |c_{wz}|<min; than c_{wz}=(c_{wz}/|_{cwz})*min

New β_{w} and γ_{w}

β_{w}=atan 2(c_{wz},ab_{wz})

ab_{wz}=cos α*a_{wz}+sin α*b_{wz}

if |c_{wz}|<min; than c_{wz}=(c_{wz}/|c_{wz})*min

γ_{w}=atan 2(a_{βx},b_{βx})

a_{βx}=cos α*cos β*a_{wx}+sin α*cos β*b_{wx}−sin β*c_{wx}

b_{βx}=−sin α*a_{wx}+cos α*b_{wx}

if |a_{βx}|<min; than a_{βx}=(a_{βx}/|a_{βx}|)*min

Convert Q_{W} to Q_{J} for Use with Jacobian

B_{Wx}=β_{w}*cos(α_{w})

B_{Wy}=β_{w}*sin(α_{w})

Q_{J}=(X_{W},Y_{W},Z_{W},β_{Wx},β_{Wy})

Numerical Jacobian:

To solve the unique Q_{J} for a deviation in joint variable (αᵢ, βᵢ, Sᵢ), one at a time, deviate each variable in every Segment by using the Transformation matrix. Then combine the resultant Q_{J} vectors to form a numeric Jacobian. By using small single variable deviations from the current joint space, a localized Jacobean can be obtained. This Jacobean can be used in several ways to iteratively find a solution for segment joint variables to a desired world space position, Q_{d}. Preferable the Jacobean is invertible in which case the difference vector between the current and desired world position can be multiplied by the inverse Jacobian J⁻¹ to iteratively approach a correct joint space variables. Repeat this process until the Forward Kinematics calculates a position vector equivalent to Q_{d} within a minimum error. Check Joint Space results (αᵢ, βᵢ, Sᵢ for i=0, 1, 2, . . . , n) for accuracy, solvability, workspace limitations and errors.

J⁻¹*J=I

αᵢ>=−360° & αᵢ<=360°

βᵢ>0 min & βᵢ<βₘₐₓ

βi≠0° or 180° (or βₘₐₓ)

β_{w}≠0° or 180°

Sᵢ>Sₘᵢₙ; Sᵢ<Sₘₐₓ

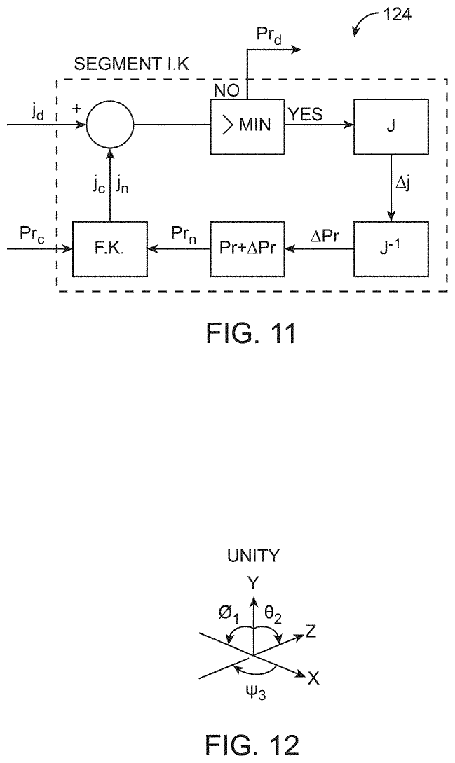

Referring now to FIG. 11, a lumen pressure flow chart 120 is shown that can be used by a processor of the robotic system to determine pressures associated with the desired movement or position of an actual or virtual catheter.

Solving for Segment Balloon Array End Coordinates:

A balloon array is a set of fluidically connected balloons along one side of a segment. Find end coordinates for each balloon array within a segment base frame. Assume balloon arrays are spaced at 120 degrees apart around the cordial axis, that the first is located on the X axis, and that the array balloons remain axially aligned through the length of the segment.

r_{A}=radius of balloon center in balloon array from cordial axis
θ=angular period of balloons within segment (120° for a 3 array segment)
θ_{A}=0; θ_{B}=120; θ_{C}=240
Arc Start points for balloon arrays:

$${P}_{0 A} = ( \begin{matrix}{r}_{A} * cos ( {θ}_{A} ) \\ {r}_{A} * sin ( {θ}_{A} ) \\ 0 \\ 1\end{matrix} ) , {P}_{0 B} = ( \begin{matrix}{r}_{A} * cos ( {θ}_{B} ) \\ {r}_{A} * sin ( {θ}_{B} ) \\ 0 \\ 1\end{matrix} ) , {P}_{0 C} = ( \begin{matrix}{r}_{A} * cos ( {θ}_{C} ) \\ {r}_{A} * sin ( {θ}_{C} ) \\ 0 \\ 1\end{matrix} )$$

Arc unit vectors for axial orientation of balloon arrays are as follows:

$${V}_{0 A} = ( \begin{matrix}0 \\ 0 \\ 1 \\ 0\end{matrix} ) , {V}_{0 B} = ( \begin{matrix}0 \\ 0 \\ 1 \\ 0\end{matrix} ) , {V}_{0 C} = ( \begin{matrix}0 \\ 0 \\ 1 \\ 0\end{matrix} ) ,$$

Use local (for one Segment) Transformation Matrix to find Balloon Array distal end coordinates.

$$T = ( \begin{matrix}\begin{matrix}c {α}^{2} * cβ + {sα}^{2} & sα * c α * ( 1 - cβ ) \\ cα * sβ & ( \text{S} \text{/β)} * cα * [ 1 - cβ ]\end{matrix} \\ \begin{matrix}sα * c α * ( 1 - cβ ) & s {α}^{2} * c β + c {α}^{2} \\ sα * sβ & ( \text{S} \text{/β)} * sα * [ 1 - cβ ]\end{matrix} \\ \begin{matrix}- cα * sβ & - sα * sβ \\ cβ & \text{(} \text{S} \text{/β)} * sβ\end{matrix} \\ \begin{matrix}0 & 0 \\ 0 & 1\end{matrix}\end{matrix} )$$

Pᵢ₁=T×P₀

P_{1A}=T×P_{0A}

P_{1B}=T×P_{0B}

P_{1C}=T×P_{0C}

For each Balloon Array set the origin at the starting points to normalize distal endpoint coordinates dP. This is helpful for solving for the Balloon Array arc, S, which follows in the Find Array Arc Lengths section below.

dPᵢ=Pᵢ₁−Pᵢ₀=(x₀,y₀,z₀) Segment Centerline Cord

dP_{A}=P_{A1}−P_{A0}=(x_{A},y_{A},z_{A}) Balloon Cord A

dP_{B}=P_{B1}−P_{B0}=(x_{B},y_{B},z_{B}) Balloon Cord B

dP_{C}=P_{C1}−P_{C0}=(x_{C},y_{C},z_{C}) Balloon Cord C

All cordial orientation vectors are equivalent.

V=(a_{iz},b_{iz},c_{iz}), for i from 1 to n.

Find Array Arc Lengths:

Referring again to FIG. 10B, solve for general Balloon Array cord lengths and radii as follows.

(α, β, S), α represent the bend direction (about z axis starting at x axis), β the bend amount (off the z axis), and S the length of an arc anchored to the origin of a reference frame.
(x, y, z) is the coordinate location of a point at the end of the arc.
r is the balloon array cord radius.

h=(x²+y²)^{1/2}

r=(h²+z²)/(2*h)

r−h=(z²−h²)/(2*h)

α=atan 2(x,y)

β=atan 2(z,r−h)

S=r*β

$$( \begin{matrix}α \\ β \\ S\end{matrix} ) = ( \begin{matrix}a tan 2 ( x , y ) \\ a tan 2 ( r - h , z ) \\ r * a tan 2 ( r - h , z )\end{matrix} )$$

Sᵢ=(hᵢ²+zᵢ²)/(2*hᵢ)*β, hᵢ=(xᵢ²+yᵢ²)^{1/2}

i (segment center cord)
Solve S for cords A, B, C, (segment center cord)
Note that segment center cord (S, β, α) is already determined.

### When β>βₘᵢₙ

S_{A}=(h_{A}²+z_{A}²)/(2*h_{A})*β, h_{A}=(x_{A}²+y_{A}²)^{1/2}

S_{B}=(h_{B}²+z_{B}²)/(2*h_{B})*β, h_{B}=(x_{B}²y_{B}²)^{1/2}

S_{C}=(h_{C}²+z_{C}²)/(2*h_{C})*β, h_{C}=(x_{C}²+y_{C}²)^{1/2}

When β=<βₘᵢₙ

S_{A}=S_{B}=S_{C}=Sᵢ

Segment Internal Load Conditions:

Segment spring force may be proportional to a spring rate with extension.

F_{S}=K_{F}*Sᵢ+F₀

Where F is the sum of the balloon forces, K_{F} is the spring constant, and F₀ is the offset force.

F₀=F_{preload}−K_{F}*Sₘᵢₙ

Where F_{preload} is the preload force at the minimum segment length Sₘᵢₙ.
Sum balloon array forces as follows:

F_{Pr}=F_{A}+F_{B}+F_{C}=A*(Pr_{A}+Pr_{B}+Pr_{C})

F_{S}=F_{Pr}

Sᵢ=(A*(Pr_{A}+Pr_{B}+Pr_{C})−F₀)/K_{F}

Segment spring torque proportional to a spring rate with bend angle.

M_{S}=K_{M}*β+M₀

β=(M_{S}−M₀)/K_{M}

Where M is the internal moment applied to the Segment, K_{M} is a angular spring constant can, and M₀ is the preload moment (compensation for no load bend).
Sum of balloon array torques:
r_{A}=radius of balloon center in balloon array from cordial axis
θ=angular period of balloons within segment (120° for a 3 array segment)
θ_{A}=0°; θ_{B}=120°; θ_{C}=240°

Mₓ=F_{A}*r_{A}*sin(θ_{A})+F_{B}*r_{A}*sin(θ_{B})+F_{C}*r_{A}*sin(θ_{C})

=(3^{1/2}/2)*A*r_{A}*(Pr_{B}−Pr_{C})

M_{y}=−F_{A}*r_{A}*cos(θ_{A})−F_{B}*r_{A}*cos(θ_{B})−F_{C}*r_{A}*cos(θ_{C})

=(½)*A*r_{A}*(Pr_{B}+Pr_{C}−2*Pr_{A})

M_{S}=(Mₓ²+M_{y}²)^{1/2}

=(((3^{1/2}/2)*A*r_{A}*(Pr_{B}−Pr_{C}))²+((½)*A*r_{A}*(Pr_{B}+Pr_{C}−2*Pr_{A}))²)^{1/2}

=(A/2)*r_{A}*(3*(Pr_{B}−Pr_{C})²+(Pr_{B}+Pr_{C}−2*Pr_{A})²)^{1/2}

=(A/2)*r_{A}*((3*Pr_{B}²−6*Pr_{B}*Pr_{C}+3*Pr_{C}²)+(Pr_{B}²+Pr_{B}*Pr_{C}−2*Pr_{A}*Pr_{B}+Pr_{B}*Pr_{C}+Pr_{C}²−2*Pr_{A}*Pr_{C}−2*Pr_{A}*Pr_{B}−2*Pr_{A}*Pr_{C}+4*pᵣ_{_{A}}²))^{1/2}

=(A/2)*r_{A}*(4*Pr_{B}²−4*Pr_{B}*Pr_{C}+4*Pr_{C}²−4*Pr_{A}*Pr_{B}−4*Pr_{A}*Pr_{C}+4*pᵣ_{_{A}}²)^{1/2}

=A*r_{A}*(Pr_{B}²−Pr_{B}*Pr_{C}+Pr_{C}²−Pr_{A}*Pr_{B}−Pr_{A}*Pr_{C}+pr_{A}²)^{1/2}

=A*r_{A}*(Pr_{A}²+Pr_{B}²+Pr_{C}²−Pr_{A}*Pr_{B}−Pr_{B}*Pr_{C}−Pr_{C}*Pr_{A})^{1/2}

M_{Pr}=M_{S}

β=[A*r_{A}*(Pr_{A}²+Pr_{B}²+Pr_{C}²−Pr_{A}*Pr_{B}−Pr_{B}*Pr_{C}−Pr_{C}*Pr_{A})^{1/2}−M₀]/K_{M}

Moment direction angle (α):

Mₓ=(3^{1/2}/2)*A*r_{A}*(Pr_{B}−Pr_{C})

M_{y}=(½)*A*r_{A}*(Pr_{B}+Pr_{C}−2*Pr_{A})

cos(α)=M_{y}/M_{S}

sin(α)=−Mₓ/M_{S}

β=(M_{S}−M₀)/K_{M}

βₓ=[(M_{S}−M₀)/K_{M}]*cos(α)=[(M_{S}−M₀)/K_{M}]*M_{y}/M_{S}

=[1−(M₀/M_{S})]*M_{y}/K_{M}

=[1−(M₀/A*r_{A}*(Pr_{A}²+Pr_{B}²+Pr_{C}²−Pr_{A}*Pr_{B}−Pr_{B}*Pr_{C}−Pr_{C}*Pr_{A})^{1/2})]*(½)*A*r_{A}*(Pr_{B}++Pr_{C}−2*Pr_{A})/K_{M}

β_{y}=−[(M_{S}−M₀)/K_{M}]*sin(α)=−[(M_{S}−M₀)/K_{M}]*Mₓ/M_{S}

=−[1−(M₀/M_{S})]*Mₓ/K_{M}

=−[1−(M₀/A*r_{A}*(Pr_{A}²+Pr_{B}²+Pr_{C}²−Pr_{A}*Pr_{B}−Pr_{B}*Pr_{C}−Pr_{C}*Pr_{A})^{1/2}))]*(3^{1/2}/2)*A*r_{A}*(Pr_{B}−Pr_{C})/K_{M}

### With M₀=0,

βₓ=(½)*A*r_{A}*(Pr_{B}+Pr_{C}−2*Pr_{A})/K_{M}

β_{y}=−(3^{1/2}/2)*A*r_{A}*(Pr_{B}−Pr_{C})/K_{M}

α=atan 2(βₓ,β_{y}); Note the minus sign for Mx is applied for matching direction.

α=atan 2((Pr_{B}+Pr_{C}−2*Pr_{A}),−1.73205*(Pr_{B}−Pr_{C}))

if (Pr_{B}−Pr_{C}|<min) & (Pr_{B}+Pr_{C}−2*Pr_{A}|<min); than α=0

Solve for P_{A}, P_{B}, and P_{C} using the following three equations and a Jacobian. Pressures should meet these conditions to solve.

|Pr_{A}—Pr_{B}|+|Pr_{B}−Pr_{C}|+|Pr_{C}−Pr_{A}|<MIN_{DIfference}; related to |β_{Calc}|>βₘᵢₙ

Pr_{A},Pr_{B},Pr_{C}>Min_{Pressure}

### Setting up Input α_{Desired} for Jacobian

IF(α_{Desired}>180°,α_{Desired}−360°,IF(α_{Desired}<−180°,α_{Desired}+360°,α_{Desired}))

Find Segment position with:

S_{Calc}=[A*(Pr_{A}+Pr_{B}+Pr_{C})−F₀]/K_{F}

βₓ=(½)*A*r_{A}*(Pr_{B}+Pr_{C}−2*Pr_{A})/K_{M}

β_{y}=−(3^{1/2}/2)*A*r_{A}*(Pr_{B}−Pr_{C})/K_{M}

### Find Pressures:

βₓ=−(3^{1/2}/2)*A*r_{A}*(Pr_{B}−Pr_{C})/K_{M}

Pr_{C}=Pr_{B}+2*(β_{y}/3^{1/2})*K_{M}/(A*r_{A})

βₓ=(½)*A*r_{A}*(Pr_{B}+Pr_{C}−2*Pr_{A})/K_{M}

2*βₓ*K_{M}/(A*r_{A})=Pr_{B}+Pr_{B}+2*(β₃/3^{1/2})*K_{M}/(A*r_{A})−2*Pr_{A}

2*βₓ*K_{M}/(A*r_{A})+2*Pr_{A}=2*Pr_{B}+2*(β_{y}/3^{1/2})*K_{M}/(A*r_{A})

Pr_{B}=Pr_{A}+βₓ*K_{M}/(A*r_{A})−(β_{y}/3^{1/2})*K_{M}/(A*r_{A})

Pr_{B}=Pr_{A}+(βₓ−β_{y}/3^{1/2})*K_{M}/(A*r_{A})

S_{Calc}=[A*(Pr_{A}+Pr_{B}+Pr_{C})−F₀]/K_{F}

(S_{Calc}*K_{F}+F₀)/A=Pr_{A}+Pr_{B}+Pr_{B}+2*(β_{y}/3^{1/2})*K_{M}/(A*r_{A})

(S_{Calc}*K_{F}+F₀)/A=Pr_{A}+2*[Pr_{A}+(βₓ−β_{y}/3^{1/2})*K_{M}/(A*r_{A})]+2*(β_{y}/3^{1/2})*K_{M}/(A*r_{A})

(S_{Calc}*K_{F}+F₀)/A=3*Pr_{A}+[2*(βₓ−β_{y}/3^{1/2})+2*(β_{y}/3^{1/2})]*K_{M}/(A*r_{A})

(S_{Calc}*K_{F}+F₀)/A=3*Pr_{A}+2*βₓ*K_{M}/(A*r_{A})

Pr_{A}=(S_{Calc}*K_{F}+F₀)/(3*A)−2*βₓ*K_{M}/(3*A*r_{A})

Pr_{B}=Pr_{A}+(βₓ−β_{y}/3^{1/2})*K_{M}/(A*r_{A})

=(S_{Calc}*K_{F}+F₀)/(3*A)−2*βₓ*K_{M}/(3*A*r_{A})+3*(βₓ−β_{y}/3^{1/2})*K_{M}/(3*A*r_{A})

Pr_{B}=(S_{Calc}*K_{F}+F₀)/(3*A)+(βₓ−3*β_{y}/3^{1/2})*K_{M}/(3*A*r_{A})

Pr_{C}=Pr_{B}+2*(β_{y}/3^{1/2})*K_{M}/(A*r_{A})

Pr_{C}=(S_{Calc}*K_{F}+F₀)/(3*A)+(βₓ−3*β_{y}/3^{1/2})*K_{M}/(3*A*r_{A})+6*(β_{y}/3^{1/2})*K_{M}/(3*A*r_{A})

Pr_{C}=(S_{Calc}*K_{F}+F₀)/(3*A)+(βₓ+3*β_{y}/3^{1/2})*K_{M}/(3*A*r_{A})

Or

S_{Calc}=[A*(Pr_{A}+Pr_{B}+Pr_{C})−F₀]/K_{F}

β_{Calc}=[A*r_{A}*(pr_{A}²+Pr_{B}²+Pr_{C}²−Pr_{A}*Pr_{B}−Pr_{B}*Pr_{C}−Pr_{C}*Pr_{A})^{1/2}−M₀]/K_{M}

α_{Calc}=atan 2((Pr_{B}+Pr_{C}−2*Pr_{A}),−1.73205*(Pr_{B}−Pr_{C}))

IF(|α_{Calc}|>45° AND |α_{Calc}|<315°,

IF α_{Calc}>0°,IF(α_{Desired}<0°,α_{Desired}+360°,α_{Desired}),

IF(α_{Desired}>0°,α_{Desired}−360°,α_{Desired})),α_{Desired})

Setup Inverse Jacobian to solve for pressures and repeat until joint (j) variable errors meet minimum condition. Check that α, β, and S meet viable solutions.

j_{calc}−j_{desired}<jₑᵣᵣₒᵣ

|α_{Calc}|<360°

β_{Calc}>β_{Min} (some amount greater than Zero),

β_{Calc}<β_{Max} (may be 180° or 360°)

S_{Calc}>S_{Min} & <S_{Max}

Solve for segment force and moment

F=K_{F}*Sᵢ+F₀

F=A*(Pr_{A}+Pr_{B}+Pr_{C})

M=K_{M}*β+M₀

M=A*r_{A}*(Pr_{A}²+Pr_{B}²+Pr_{C}²−Pr_{A}*Pr_{B}−Pr_{B}*Pr_{C}−Pr_{C}*Pr_{A}))^{1/2}

### Solve for Segment Length and Angle

S=(F−F₀)/K_{F} Check for equivalency

β=(M−M₀)/K_{M} Check for equivalency

Communications Between Robot Articulation Controller Module And Simulation/Display Processor Module

Referring now to FIGS. 9 and 12, the module used to control movement of the actual catheter in the surgical workspace may have a 3-D workspace with a first reference frame (sometimes referred to herein as the ROBOT reference frame), while the simulation module used to calculate virtual movement of a virtual catheter in a virtual workspace may use a second reference frame (such as a simulation reference frame, sometimes referred to herein as the UNITY™ reference frame) that is different from the first reference frame. For example, the ROBOT reference frame may be a right-hand-rule reference frame with the positive Z orientation being vertically upward, while the virtual or UNITY™ reference frame may be a left-hand-rule reference frame with the positive Y axis being upward. The calculations performed in these different environments may also have differences, such as relying predominantly or entirely on Euler angles and transformation matrices in the ROBOT calculations and relying more predominantly or even entirely on quaternions and related vector manipulations in the virtual reference frame, with any Euler angle-related transformations optionally based on a different set of rotation axes, different rotation orientations, and/or different order of rotations. Nonetheless, there may be advantages to sending data in both directions (from the virtual environment to the actual robot, and from the actual robot to the virtual environment) during a procedure so as to provide enhanced correlation between the virtual and actual poses and system performance. Hence, communications between the modules handling the virtual and actual environments may be provided as follows.

Coordinates

Robot and Unity™ or simulation module coordinate systems are different. To swap coordinates relabel the Robot Z axis to be the Unity™ Y axis and the Robot Y axis to be the Unity™ Z. At the base of the first segment the catheter axis points along the Robot Z axis and in the Unity™ Y axis. The base segment starts vertical in both coordinate frames.

Angles

Robot and Unity™ coordinate angles are measured in opposite directions. When viewed with the axis of rotation pointing towards the observer, the Robot angles are measured counter clockwise while the Unity™ angles are measured clockwise.

Rotation Types

The Robot rotations act intrinsically, which means the second and third rotation is about a coordinate system that moves with the object in prior rotations. The Unity™ rotations act extrinsically, which means the all rotations acting on an object are about a fixed coordinate system. The axis defining a rotation for intrinsic rotations incudes the number of apostrophes to indicate the sequence.

Axis of Rotation

The Robot rotations rotate about axes z-y′-z″, in this order and about rotating coordinate frames. The Unity™ rotations rotate about axes Z-X-Y, in this order, about a fixed coordinate frame.

Rotation Nomenclature

The Robot segments rotation angles are labeled with alpha (α), beta (β), and gamma (γ) and define the segments angles of rotation about the rotating frames axes z-y′-z″ of the Robot base coordinates. The Unity™ segment rotation angles are labeled with phi (φ), theta (θ), and psi (w) and define the segments angles of rotation about the fixed frame axes Z-X-Y of the Unity™-Robot base coordinates.

Position Nomenclature

The Robot segments positions are denoted by lower case x, y, and z and define the location of the segments distal end using the Robot base coordinates. The Unity™ segment positions are denoted by upper case X, Y, Z and define the location of the segments distal end within the Unity™-Robot base coordinates.

Command Protocol

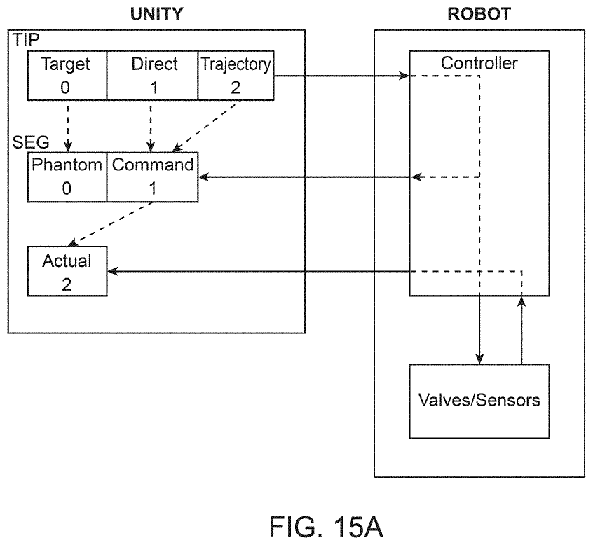

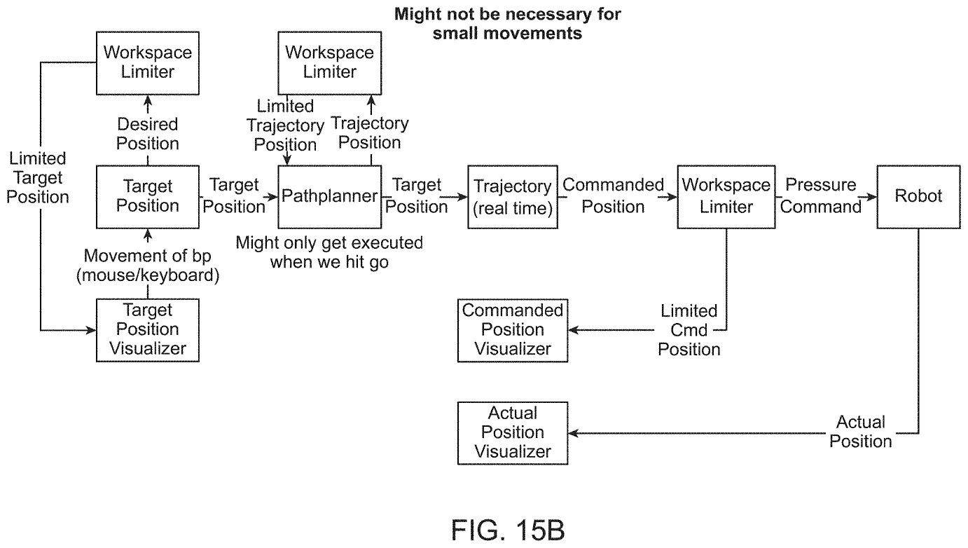

The Command input comes from Unity™ and is delivered to the Moray Controller in Unity™ coordinates. Command inputs may be incremental changes to affect the robot tip position and orientation and based on a coordinate system attached to a robot tip at the distal end of the Unity™ segments. Alternatively, the command inputs may be the absolute position and orientation of the robot tip based on the coordinate systems attached to the base at the proximal end of the Unity™ segments. As can be understood with reference to FIGS. 15A and 15B, there are two command vector types, a direct command vector which moves the robot immediately and a target command which moves a target or virtual robot in Unity™. The target robot defines the end of a trajectory for the movement or direct command to follow upon user direction. Each command vector includes 6 variables which define 6 degrees of freedom to full specify the tip position. The command protocol is defined by two vectors including a Command and a Target vector.

The following represents the Unity™ generated data set (and nomenclature) from Unity™ to the Robot Controller.
CTX;CTY;CTZ;CTφ;CTθ;CTψ;TTX;TTY;TTZ;TTφ;TTθ;TTψ<CR>

Telemetry Protocol

The telemetry input comes from the robot controller and is delivered to Unity™ in Unity™ Euler coordinates. Telemetry inputs relate the position and orientation of the robot segment ends based on a coordinate system at the base of the most proximal segment. There are three telemetry vector types, a command telemetry vector, an actual telemetry vector and a target telemetry vector. The command telemetry is that which is being asked for by the command input, the actual telemetry is the measured segment positions, and the target telemetry reflects the phantom segments position. Each telemetry vector holds 14 variables which includes two manual (sensed) catheter base inputs and two segments end conditions (6 values for each segment). The telemetry protocol has 43 values and starts with a packet count number, followed by the command, actual, and target telemetry vectors.

The following represents the Robot Controller generated data set (and nomenclature) from Robot Controller to Unity™
KNN;CY0;Cv0;CX1;CY1;CZ1;Cφ1;Cθ1;Cψ1;CX2;CY2;CZ2;Cφ2;Cθ2;Cψ2;
   AY0;Aψ0;AX1;AY1;AZ1;Aφ1;Aθ1;Aψ1;AX2;AY2;AZ2;Aφ2;Aθ2;Aψ2;
   TY0;Tψ0;TX1;TY1;TZ1;Tφ1;Tθ1;Tψ1;TX2;TY2;TZ2;Tφ2;Tθ2;Tψ2<CR>
      Note: Future systems that have more than two segments will benefit from additional telemetry vectors for each additional segment.

Command and Telemetry Variables

The nomenclature for this data set include the following:

| CT - | Command Tip | (Command Tip Vector) |
|---|---|---|
| TT - | Target Tip | (Phantom Tip Vector) |
| C - | Command Segments | (Command Segment Vector) |
| A - | Actual Segments | (Actual Robot Segment Vector) |
| T - | Target Segments | (Phantom Segment Vector) |
| K - | Packet | (Packet count number) |
| NN - | Number | |
| X - | X millimeters | (X position telemetry) |
| Y - | Y millimeters | (Y position telemetry) |
| Z - | Z millimeters | (X position telemetry) |
| φ - | φ degrees | (φ angle CCW about Unity ™ Z axis) |
| θ - | θ degrees | (θ angle CCW about Unity ™ X axis) |
| ψ - | ψ degrees | (ψ angle CCW about Unity ™ Y axis) |
| 0 - | Base | (Base defined parameter - axial and rotation) |
| 1 - | Segment 1 | (Segment number 1 parameter) |
| 2 - | Segment 2 | (Segment number 2 parameter) |

Converting Robot to Unity™ Coordinates

For the purpose of finding to the Unity™ rotation angles, the Robot coordinate axes will be used. At the end of the computation the Z and Y axis are be switched to sync with the Unity™ coordinate system. Therefore, the Unity™ rotations (using the Robot axes) have the base of the segments in plane with the Y-X axes. The extrinsic rotations are therefore about Y-X-Z (formerly Z-X-Y with the Unity™ axes) with -θ-ψ representing associated rotations. When converting from an extrinsic to an intrinsic rotational sequence the order of rotation is reversed. Therefore, an equivalent intrinsic system will have rotations about z-x′-y″ with ψ-θ-φ, in this order.

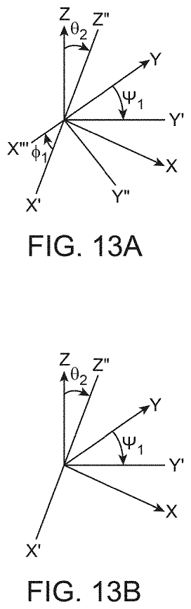

Referring now to FIG. 13A, the Y axis is the last rotation axis; therefore, the Y unit vector is not affected by the last rotation and can be used to find psy (ψ) and theta (θ).

Referring once again to FIG. 12, Unity™ coordinates align the proximal end of the first segment along the Y axis and have extrinsic rotations about Z-X-Y with φ-θ-ψ. For position conversion from the actual Robot to the virtual or Unity™ Robot, switch the Z and Y position values.

Converting Unity™ to Robot Coordinates

Referring now to FIG. 13B, Unity™ provides user input data to the Robot in the form of a change in the Tip position and orientation, q. The Tip coordinates coordinate systems differ between Unity™ and the Robot in the same way as described above, the Y and Z axis are swapped, and they orient with a different type and sequence of rotation.

The angles in this case represent the deviation from the last tip position. Solving the rotational matrix and finding the Robot angles directly solves for the Robot Tip deviations.

For position conversion from the Unity™ (input or virtual) Robot to the actual Robot, switch the Z and Y position values.

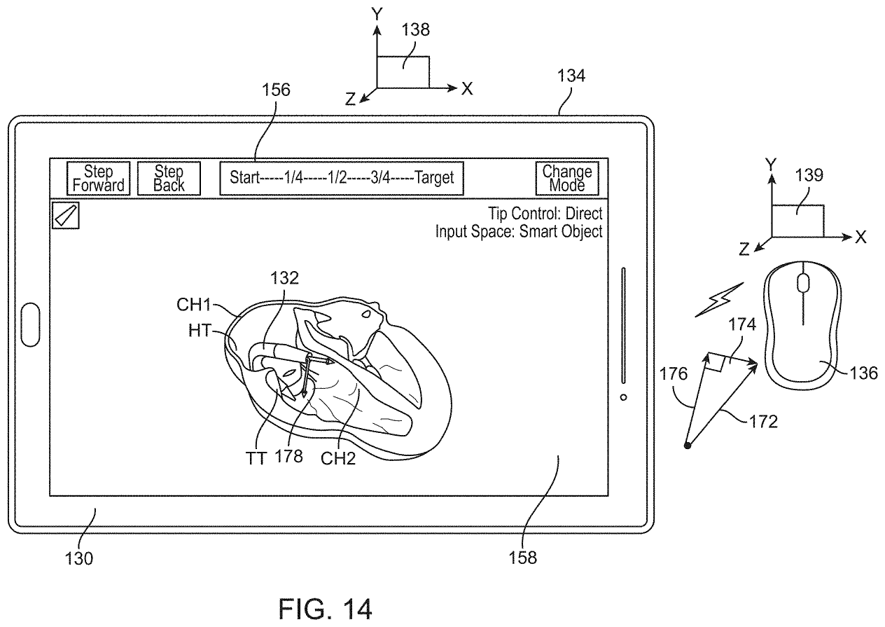

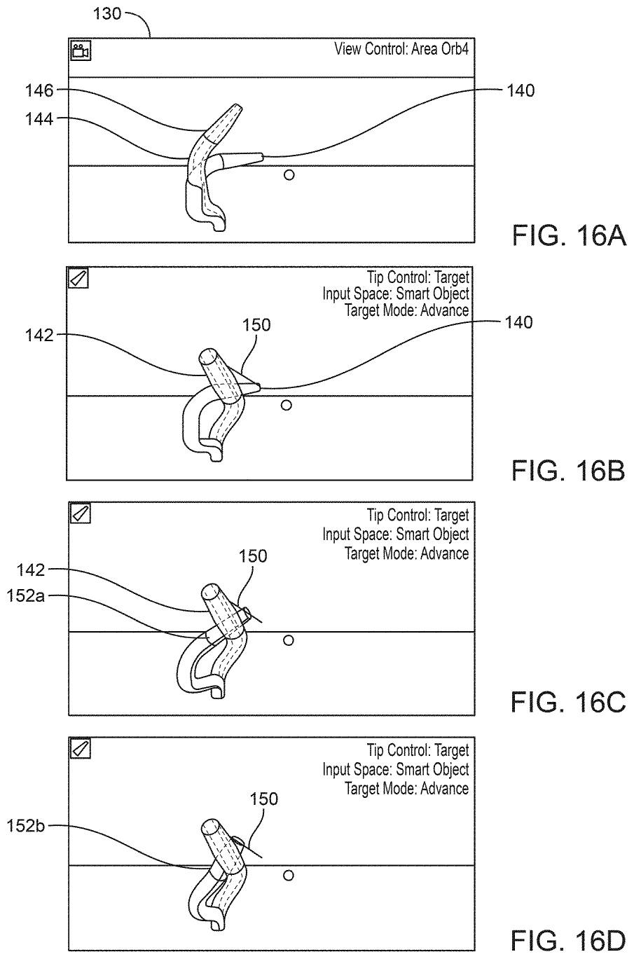

Referring now to FIGS. 14 and 16A-16D, a method for using a computer controlled flexible catheter system for aligning a virtual and/or actual therapeutic or diagnostic tool with a target tissue can be understood. As seen in a display 130, an image of heart tissue adjacent an internal site (here including one or more chamber of the heart CH1, CH2) in a patient is being shown, with the heart tissue image typically being acquired prior to or during a procedure, or simply being based on a model. Target tissue TT may comprise a tissue structure of the tricuspid or mitral valve, a septum or other wall bordering a chamber of the heart, an os of a body lumen, or the like. An image of a catheter having an elongate body 132 has been inserted into the patient or superimposed on the tissue image, the elongate body having a receptacle to support the tool. The position and orientation of the receptacle define a first pose of the tool within the 3-D internal surgical site. Display 130 may comprise a surgical monitor, a standard desktop computer monitor, or a display of a notebook computer, with the exemplary display in this embodiment comprising a touchscreen of a tablet 134. The touchscreen may be used for both input into and output from the data processing system. An at least 2-D input device 136 separate from the touchscreen may optionally be used for input with or instead of the touchscreen, and a display housed in a structure separate from the tablet may be coupled to a processor of the tablet for use with or instead of display 130.

Referring still to FIG. 14, display 130 generally has an image display plane 138 with a first or lateral orientation X and a second or transverse orientation Y. A third or Z orientation may extend into display 130 (away from the user) in a left-hand or Unity™ display coordinate system. Input commands on a touchscreen display 130 will typically comprise position changes with components in the X and Y orientations. Similarly, input commands sensed by input device 136 may have X and Y component orientations. Z components of the input commands may be sensed as pinch gestures on a touchscreen, rotation of a scroll wheel of a mouse, axial movement of a 3-D input device, or the like. Note that the display coordinate system or reference frame may be referred to as the camera reference frame, and may be offset from the world coordinate system of the virtual and/or real 3D workspace. An image plane may be parallel to the display plane adjacent the tissue at a selectable distance from the display plane, or may be manipulatable by the user so as to set an offset angle between the display and image planes.

Referring now to FIGS. 16A-16C, it may be beneficial to allow the user to move a virtual receptacle around within the internal worksite to more than one candidate pose to evaluate a number of potential poses and pick a particularly desired or target pose, but without having the delay or to impose the potential trauma of actually moving the catheter to or through poses that are not ideal. Toward that end, the processor may include a module configured for receiving, from a user, input for moving the receptacle and tool of the catheter from a first pose 140 to a second pose 142 within the internal surgical site. The input may make use of a virtual receptacle, tool, and/or catheter 144 having an image that moves in display 130, with the virtual receptacle often defining one or more candidate or intermediate input poses 146 after moving from the first pose 140 and before the being moved to the desired second pose 142. Rather than treating the phantom catheter as a master-slave input during such a movement, the processor of the system may drive the actuators coupled to the catheter so that the receptacle remains fixed at the first location while the user inputs commands on the touchscreen or via another input device to identify the desired receptacle pose. Once the desired pose has been established, the processor can receive a movement command to move the receptacle. In response, the processor may transmit drive signals to a plurality of actuators so as to advance the receptacle along a trajectory 150 from the first pose toward the second pose. Advantageously, the trajectory can be independent of the intermediate, non-selected candidate input pose 146, as well as from the (potentially meandering) trajectory input by the user to and from any number of intermediate poses 146.

As can be understood with reference to FIGS. 16B-16D, when the receptacle of the catheter is in the first pose 140 and the user inputs the second pose 142 into the processor (preferably using a virtual catheter image moving in the 3D workspace), trajectory 150 from the first pose to the second pose will often comprise a relatively complex series of actuator drive signals that move the catheter body that drive the actuators in sequence to induce movement in a plurality of degrees of freedom that result in the desired change in both position and orientation of the therapeutic tool. Advantageously a quaterion-based trajectory planning module (optionally included in the input processor) may calculate the trajectory as a linear interpolation between the first and second poses, and the user may optionally manipulate the trajectory as desired to avoid deleterious tissue engagement or the like. Regardless, it will often be desirable for the user to maintain close control over advancement of the catheter along the trajectory. Toward that end, the processor may receive a movement command from the user to move along an incomplete spatial portion of trajectory 150 from the first pose 140 to the second pose 142, and to stop at an intermediate pose 152a between the first pose and the second pose. For example, the movement command may be to move a rational fraction of the trajectory, such as ¼ of the trajectory, ⅛ of the trajectory, or the like. The user may gradually or incrementally complete the trajectory in one or more portions 152a, 152b, . . . , stop after one or more spatial portions and choose a new desired target pose, or even move back along the trajectory one or more portions away from the second pose and back toward the first pose. The movement command may be entered as a series of steps (such as using forward and backward step buttons 154a, 154b of the touchscreen in FIG. 14, forward and backward arrow keys on a keyboard, or the like) or using a single continuous linear input sensor (such as a scroll wheel of mouse or linear scale 156 of a touchscreen as in FIG. 14). In response to the movement command, the processor can transmit drive signals to a plurality of actuators coupled with the elongate body so as to move the receptacle toward the intermediate pose, optionally using the motion control arrangement described above.

Referring now to FIGS. 14 and 17A-18, exemplary methods for using a planar input device (such as a touchscreen 158 of tablet 134 or a mouse input device 136) to achieve accurately controlled motion in up to three positional degrees of freedom and one, two, or three orientational degrees of freedom can be understood. More specifically, these methods and systems can be used for manipulating a real and/or virtual elongate tool 162 in a three-dimensional workspace 164. The tool has an axis 166, and may be supported by a flexible catheter or other support structure extending distally along the axis to the tool, as described above. As can also be understood from the description above, an input/output (I/O) system 111 (see FIG. 5) can be configured for showing an image of the tool in a display 168 and for receiving a two-dimensional input from a user. The I/O system will often have at least one plane (see image plane 138, and input plane 139 of FIG. 14) and the axis of the tool as shown in the tool image may have a display slope 170 along the display plane. The user may enter a movement command by moving a mouse on the input plane, by dragging a finger along touchscreen 158, or the like, thereby defining an input 172 along the input plane. A first component 174 of the input can be defined along a first axis corresponding to the tool display slope, and a second component of the input 176 can be defined along a second axis on the input plane perpendicular to the tool display slope.

To facilitate precise control over both the position and orientation of the tool in the workspace, the processor of the system may have a translation input mode and an orientation input mode. When the processor is in the orientation mode, the first component 174 of the input 172 (the portion extending along the axis 166 of the tool as shown in the image), will typically induce rotation of the tool in three-dimensional workspace 158 about a first rotational axis 178 that is parallel to the display plane 138 and perpendicular to the tool axis 166. In response to the second component 174 of the input, the processor can induce rotation of the tool and tool image about a second rotational axis 180 that is perpendicular to the tool axis 166 and also to the first rotational axis 178. Using vector notation, the first rotational axis V_{N} can be calculated from the first and second components of the input V₁, V₂ as:

$$\underline{{V}_{1} \times {V}_{2}} = {V}_{N}$$

$$\vert V 1 * V 2 \vert$$

The second rotational axis Vs can then be calculated from the inverse of the first rotational axis V_{NS} and from the axis 166 of the tool VT as follows:

V_{NS}×V_{T}=V_{S}

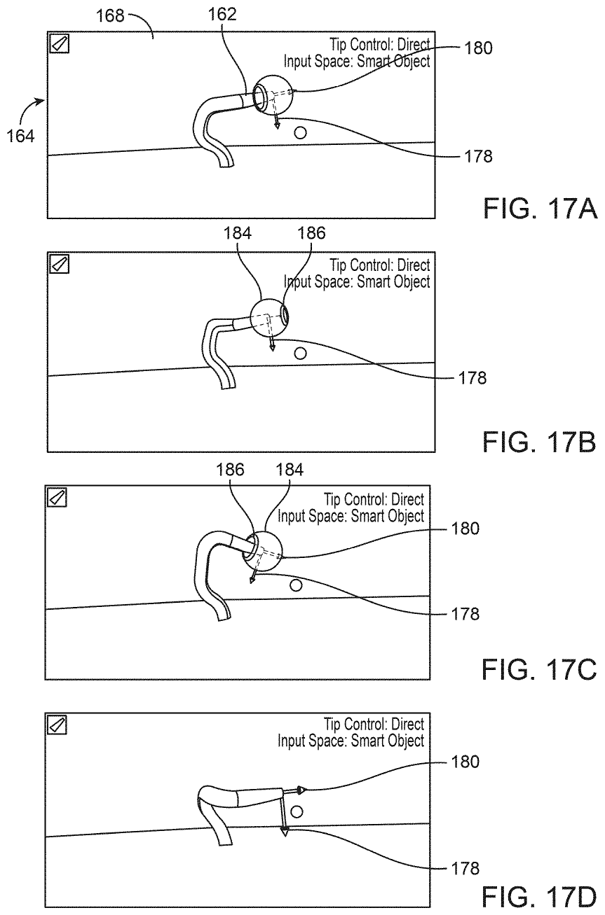

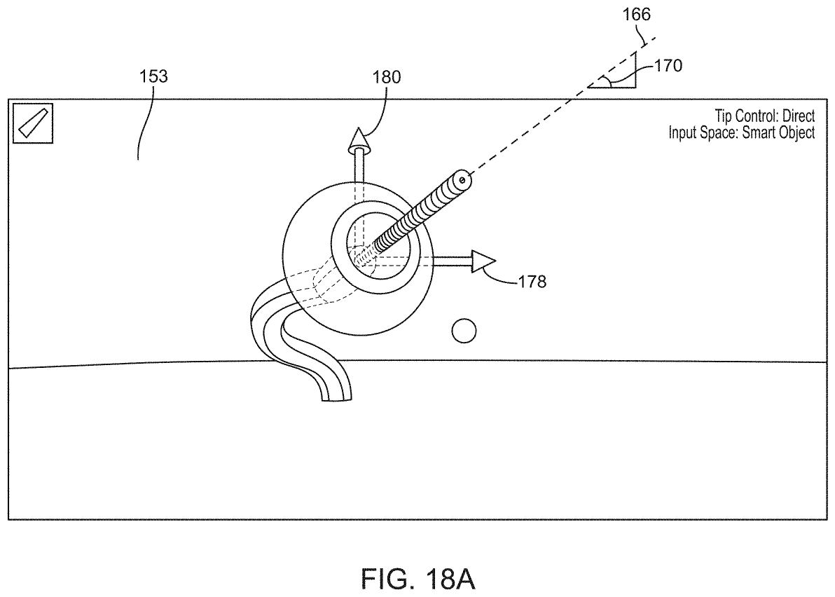

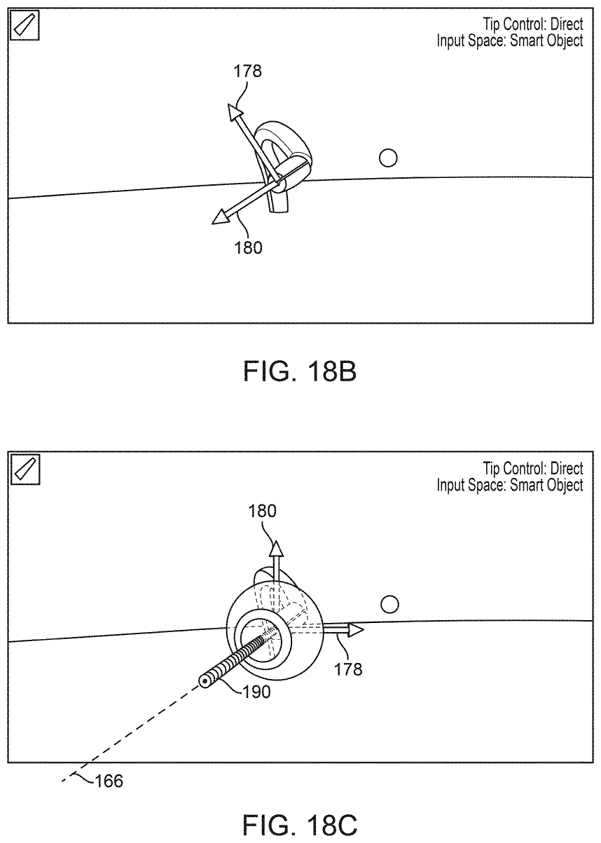

The tool axis 166 and first and second rotational axes 178, 180 will typically intersect at a spherical center of rotation at a desired location along the tool, such as at a proximal end, distal end, or mid-point of the tool. To help make the rotational movement intuitive, the processor can superimpose an image of a spherical rotation indicator such as a transparent ball 184 concentric with the center of rotation. Superimposing rotation indicia such as a concentric ring 186 encircling the tool axis 166 on the side of ball 184 oriented toward the user can further help make the orientation of rotation predictable, as input movement of the mouse or movement of the user's finger on a touchscreen in an input direction along the input plane can move the rotation indicia in the same general direction as the input movement, giving the user the impression that the input is rotating the ball about the center of rotation with the input device. The rotation indicia will preferably stay at a fixed relationship relative to the center of rotation and tool axis during a rotation of the tool, but may switch sides of the ball when a rotation increment is complete (as can be understood by comparing FIGS. 17B and 17C. As can be understood with reference to FIGS. 18A-C, when the tool axis 166 is within a desired angle range of normal to the display plane, the rotational axes 178, 180 may revert to extending along lateral and transverse display orientations (see X-Y orientations along plane 138 in FIG. 14).

When a planar input device is in use and the input processor is in an object-based translation mode, input movement along the axis slope 170 may result in translation of tool 162 in workspace 164 along the second rotational axis 180. Input command movement along the display and/or image plane perpendicular to the axis slope 170 may induce translation of tool parallel to the first rotational axis 178. Advantageous movement of the tool along the axis of the catheter when using a mouse or the like can be induced by rotating a scroll wheel. In a view-based translation mode, input command movement along the X-Y input plane 139 can induce corresponding movement of the tool along the X-Y display plane 138. Scroll wheel rotation in the view-based translation mode can induce movement into and out of the display plane (along the Z axis identified adjacent display plane 138 in FIG. 14). Selection between input modes may be performed by pushing different buttons of a mouse during input, using menus, and/or the like. Optionally, a ruler 190 having axial measurement indicia may be superimposed by the processor extending distally from the tool along axis 166 to facilitate axial measurement of tissues, alignment with and proximity of the tool to the target tissues, and the like. Similarly, lateral offset indicia such as a series of concentric measurement rings of differing radii encircling axis 166 at the center of rotation can help measure a lateral offset between the tool and tissues, the size of tissue structures, and the like.

A number of additional input and/or articulation modes may be provided. For example, the user may select a constrained motion mode, in which movement of the tool or receptacle is constrained to motion along a plane. The plane may be parallel to the display plane and the processor may maintain a separation distance between the tool and the constraint plane (which may be coincident with or near an imaging plane of the imaging system) when the planar movement mode is initiated. This can help keep the tool in view of, for example, a planar ultrasound imaging system, while facilitating movement of the tool relative to tissue structures while both remain at good imaging depths. Alternatively, the user may use the input system to position a constraint plane or other surface at a desired angle and location within the 3-D workspace. Alternative constraint surfaces may allow movement on one side of the surface and inhibit motion beyond the surface, or the like. Such constrained motion can be provided by constraining the above catheter motion arrangements with the equation for the surface, such as with the equation for a plane (aX+bY+cZ+d=0). Hence, a wide variety of alternative surface-based or volume-based movement constraints may be provided.

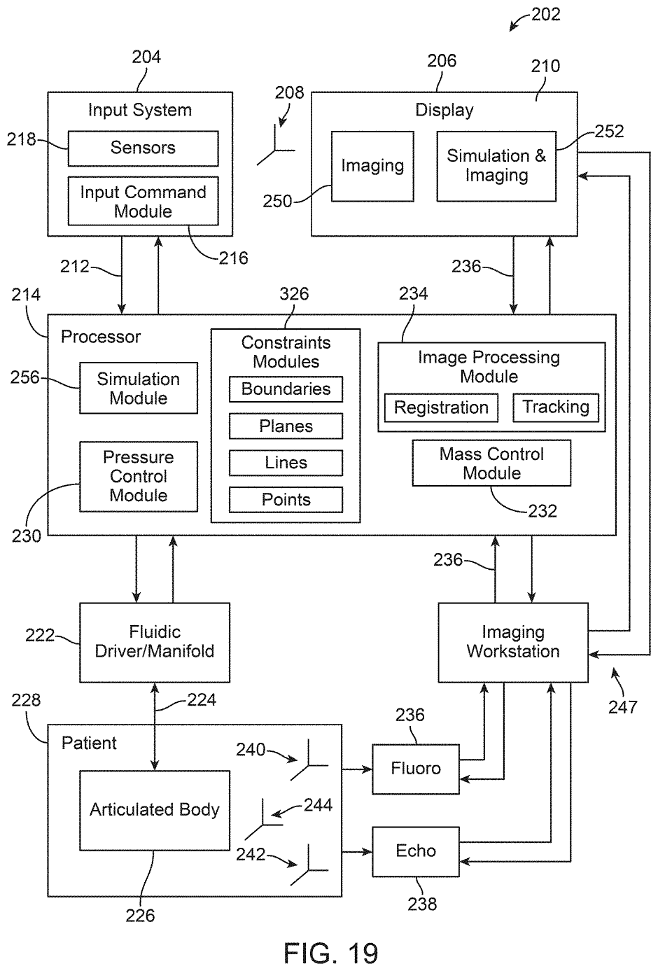

Referring now to FIG. 19, a functional block diagram schematically illustrates aspects of an exemplary data architecture of a fluid-driven structural heart therapy delivery system 202. In general terms a user inputs user commands into an input system 204 with reference to images presented by a display 206, with the input commands typically being entered in an input and display reference frame 208 which may optionally be defined in part by a plane 210 of display 206, and in part by 3D orientations of the objects and tissues represented by the displayed images. Those 3D orientations are, in turn, determined by an orientation and position of an image capture device (used to acquire the image) relative to the objects, or in the case of virtual or model objects, by the calculated camera frame. Hence, display frame 208 is sometimes referred to herein as a camera reference frame.

Referring still to FIG. 19, input commands 212 are transmitted from input system 204 to a processor 214, which may optionally transmit robotic system state data or the like back to the input system. Processor 214 includes data processing hardware, software, and/or firmware configured to perform the methods described herein, with the functional combinations of these components which work together to provide a particular data processing functionality often being described herein as a module. As noted above, the distribution of these data processing modules need not (and often will not) correspond exactly to separation of physical components of the fluid-driven robotic system. For example, an input command module 216 that generates input commands 212 in response to signals from movement sensors 218 may include software components running on a processor board of a 6 DOF input device, and may also include other software components running on a board of a desktop or notebook computer. Processor 214 sends actuator commands 220 in the form of valve commands to a fluidic driver/manifold 222 resulting in the transmission of inflation fluid 224 to the articulation balloon array of an elongate articulated body 226, thereby inducing movement of the tool receptacle and tool in the patient body 228. The pressures of the balloon inflation lumens provides a feedback signal that can be used by a pressure control module 230 of processor 214 to help determine an inflation state of the balloon array and an articulation state of the catheter or other elongate body.

As can be understood with reference to FIG. 19, along with pressure signals from the articulation balloon array, additional feedback may be employed by the data processing components of delivery system 202 to generate actuator commands. For example, a mass control module 232 of processor 214 may track an estimated mass in the subsets of balloons in the balloon array whenever the valves of the manifold open to add inflation fluid to or release inflation fluid from a balloon inflation channel, so that the absolute articulation state of the articulated body can be estimated from the inflation fluid mass and the sensed pressure of the lumen. Articulation state may also be sensed by an optical fiber sensing system having an optical fiber sensor extending along the elongate body. Still further articulation feedback signals may be generated using an image processing module 234 based on image signals 236 generated using a fluoroscopy image capture device 236, an ultrasound image capture device 238, an optical image capture device, and MM system, and/or the like. Preferably the image processing module 234 includes a registration module that determines transformations that can register fluoro image data obtained by the fluoro system 236 in a fluoro reference frame 240 with echo image data obtained by echo system 238 in an echo reference from 242 so as to provide feedback on catheter movement in a unified catheter reference frame 244. Such registration of the fluoro and echo data may make use of known and/or commercially available data fusion technologies, including those commercialized by Philips, Siemens, and/or General Electric, and/or registration of the catheter position and orientation may make use of known catheter voxel segmentation such as those described by Yang et al. in an article entitled “Catheter segmentation in three-dimensional ultrasound images by feature fusion and model fitting,” Journal of Medical Imaging 6(1), 015001 (January-March 2019), or as the processor 214 of system 202 will often have access to a-priori knowledge of the catheter structure and current knowledge of its bend state, to any of the earlier known segmentation approaches. As such registration may take more than a second (typically taking more than a 10 seconds, or even more than a minute), tracking ongoing movements of the registered catheter from streaming image data intermittently or in real time may employ a faster tracking technique, such as that described by Nadeau et al. in the article entitled “Target Tracking in 3D Ultrasound Volumes by Direct Visual Servoing,” The Hamlyn Symposium on Medical Robotics (2012).

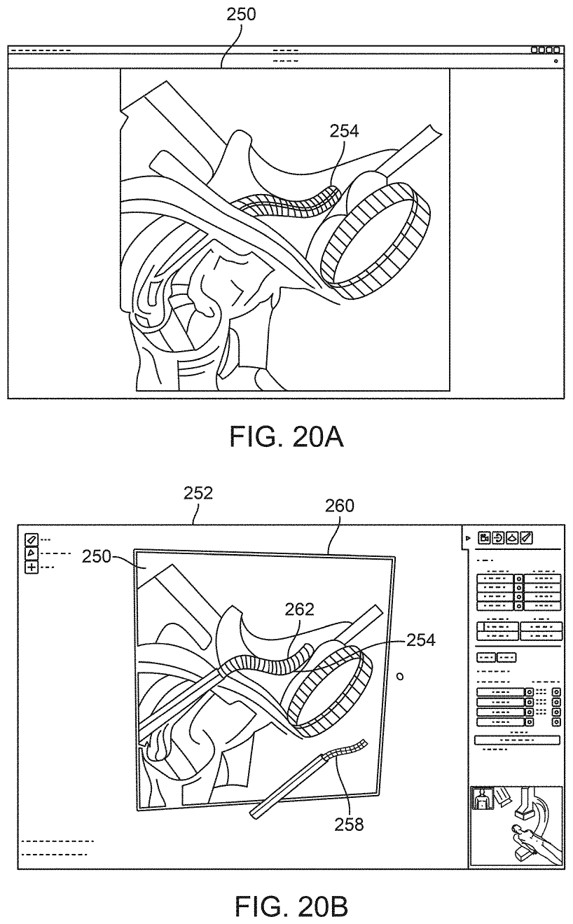

Referring now to FIGS. 19, 20A, and 20B, display 206 will often present acquired images 250 and auxiliary images 252, the auxiliary images optionally comprising virtual images showing a simulation or graphical model of the articulated body (as discussed above) and/or an augmented image including a simulation of the articulated body superimposed on an acquired image. As seen in the simulated intracardiac fluoroscopic acquired image 250 of FIG. 20A (actually an optical image taken in a benchtop model), the image data acquired by the optical imaging system is used to present a real-time 2D image of articulated body 254 and surrounding tissues. This image data is combined with 3D model data generated by a simulation module 256 of processor 214 so that auxiliary image 252 includes one, two, or all of: i) a 3D image of the model articulated elongate body 258, ii) at least a portion of the acquired real-time 2D image 250 on an associated image plane 260 in the display space 208, and iii) a 2D projection 262 of the model onto the 2D image plane superimposed on the acquired 2D image. The superimposed 2D model or virtual image 262 and acquired image of the articulated body 254 can help the user or an automated image processing system to register, track, and verify alignment of the model with the actual catheter, so that the augmented reality display 252, often with acquired display 250, allows enhanced image guidance when aligning a tool supported by the catheter with a target tissue.

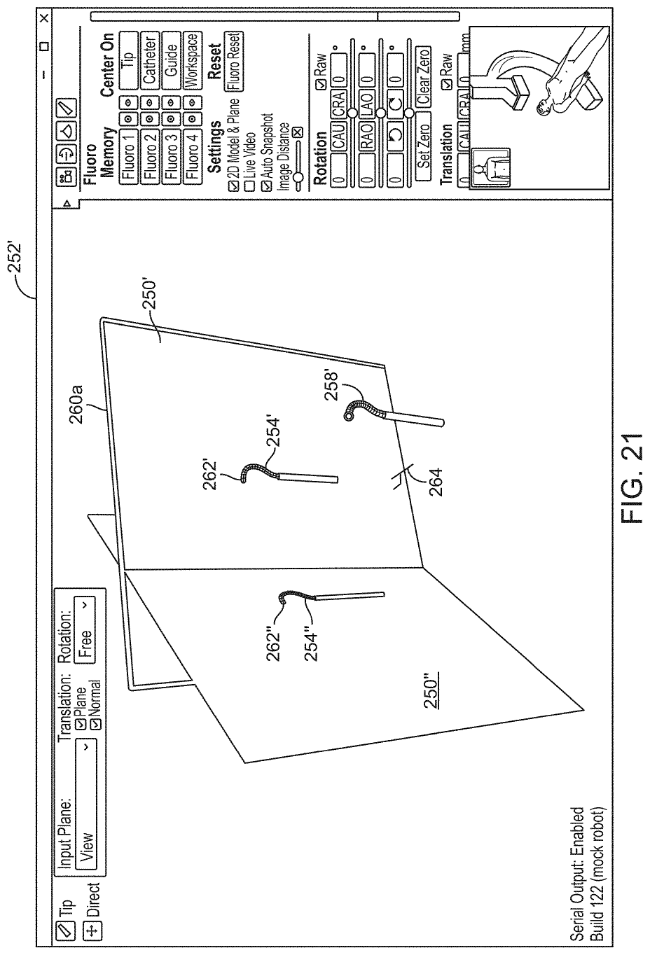

Referring now to FIGS. 19, 20B, and 21, auxiliary image 252 will often comprise a hybrid 2D/3D image presenting a 2D image elements included within a 3D image environment. More specifically, as noted above some or all of acquired image 250, 250′ is included in auxiliary image 252, 252′, with the acquired image data being manipulated so as to appear on an image plane 260, 260a at a desired angle and location within the display space 208. A 3D virtual image of a model of the catheter or other elongate body 258, 258′ is also presented, and while the acquired image remains a 2D image, it can be scaled, tilted, etc. and/or the 3D model can be oriented (and optionally scaled) so that an orientation of the 2D acquired image of the catheter corresponds to a projection of the 3D model onto associated image plane 260, 260a, preferably along a normal to the image plane 264. As noted above, a 2D projection of the 3D virtual image onto the image plane may also be included. As can be understood with reference to FIG. 21, a second acquired image 250″ at a different orientation than the first acquired image 250 may be presented on a second image plane 260b, typically including another 2D image of the catheter 254″ and/or a second 2D projection 262″ of the 3D virtual catheter model 258′ onto the image plane. Note that one or both of the capture images may be generated from recorded image data (optionally being a recorded still image or a recorded video clip, such as from a single plane fluoro system with the C-arm at a prior offset angle) so that the recorded image of the catheter may not move in real time, but can still provide beneficial image guidance for alignment of a virtual catheter relative tissues.

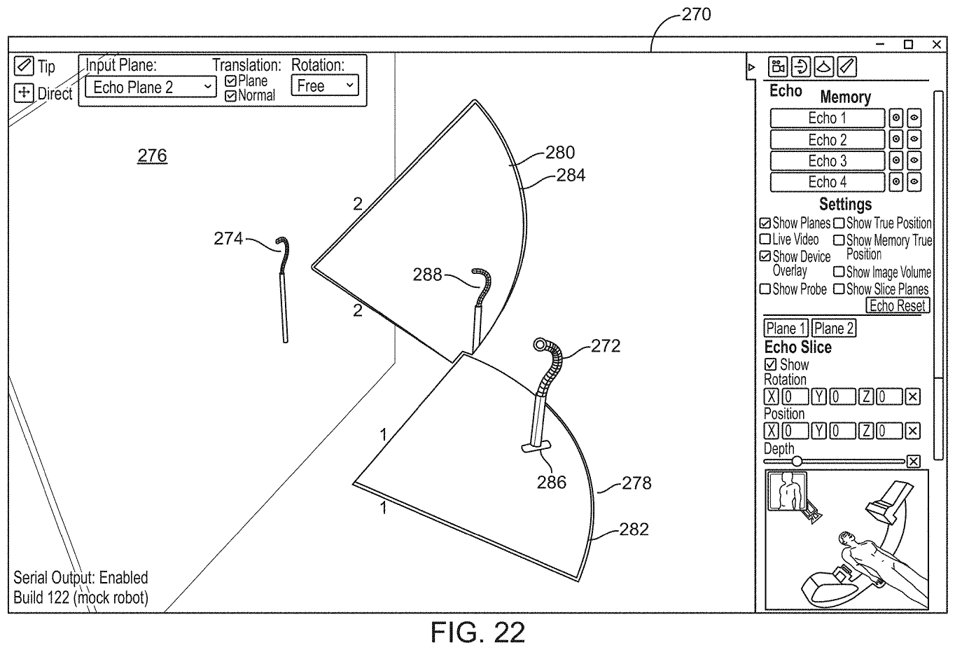

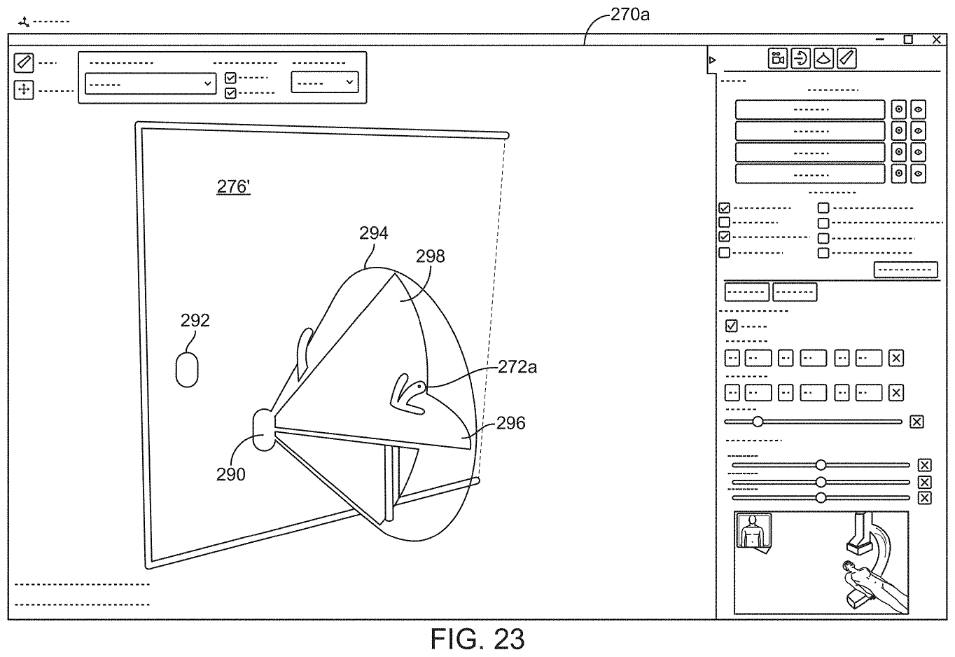

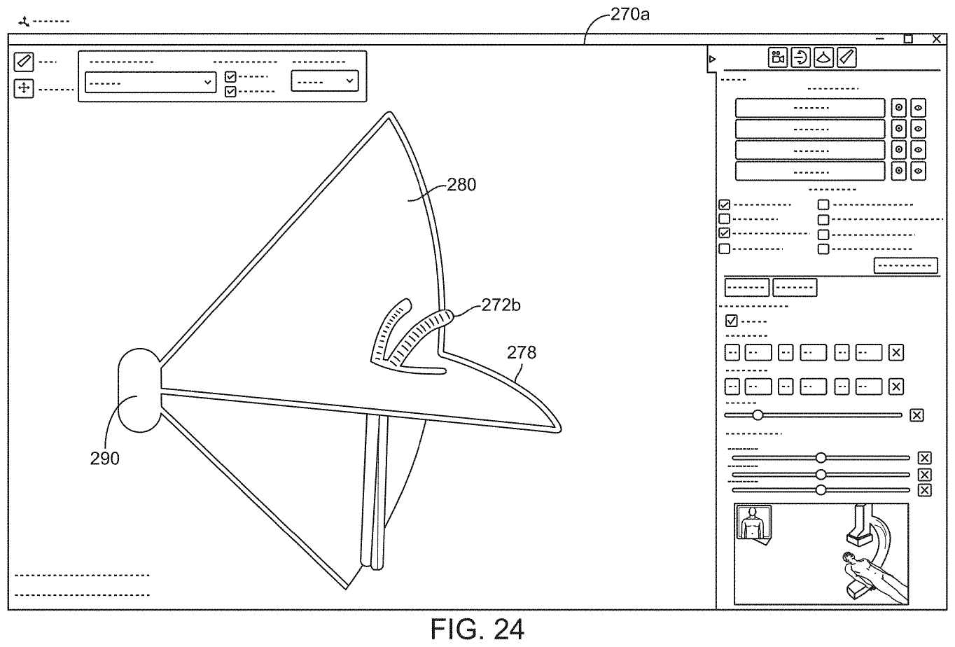

Referring now to FIGS. 22-25, alternative hybrid 2D/3D transesophageal echocardiography (TEE) or intracardiac echocardiography (ICE) images 270, 270a, 270b, 270c includes many elements related to those described above, including a 3D virtual model image and an associated 2D virtual model superimposed projection 274 onto an image plane of an acquired fluoroscopic image 276. For example, as seen in FIG. 22, hybrid TEE or ICE image 270 also includes first and second acquired 2D echo images 278, 280 on associated echo planes 282, 284, with projected 2D virtual model images 286288 superimposed on the acquired echo images along normals to the planes. As schematically shown in FIGS. 23 and 24, echo images 278, 280 are acquired using a TEE or ICE probe 290, and an image 292 of TEE or ICE probe 290 will often be seen in the acquired fluoroscopic image 276′. This facilitates the use of known data fusion techniques for registering the image data from the fluoro system with the image data from the echo system. TEE or ICE probe 290 will optionally comprise a volumetric TEE or ICE probe acquiring image data throughout a volume 294. Optionally, 3D acquired images of the catheter can be presented in hybrid TEE image 270 using such volumetric TEE capabilities. Nonetheless, image guidance for alignment of the catheter-supported tool may benefit from presenting acquired echo image slices obtained from intersecting echo image planes 296, 298 within the echo data acquisition volume 294. To facilitate clear viewing of the acquired 2D echo images (often augmented with 2D virtual catheter images superimposed thereon) the echo image display planes 282, 284 can be offset from the 3D catheter virtual model 272, 272c, optionally along normals of the associated image planes.

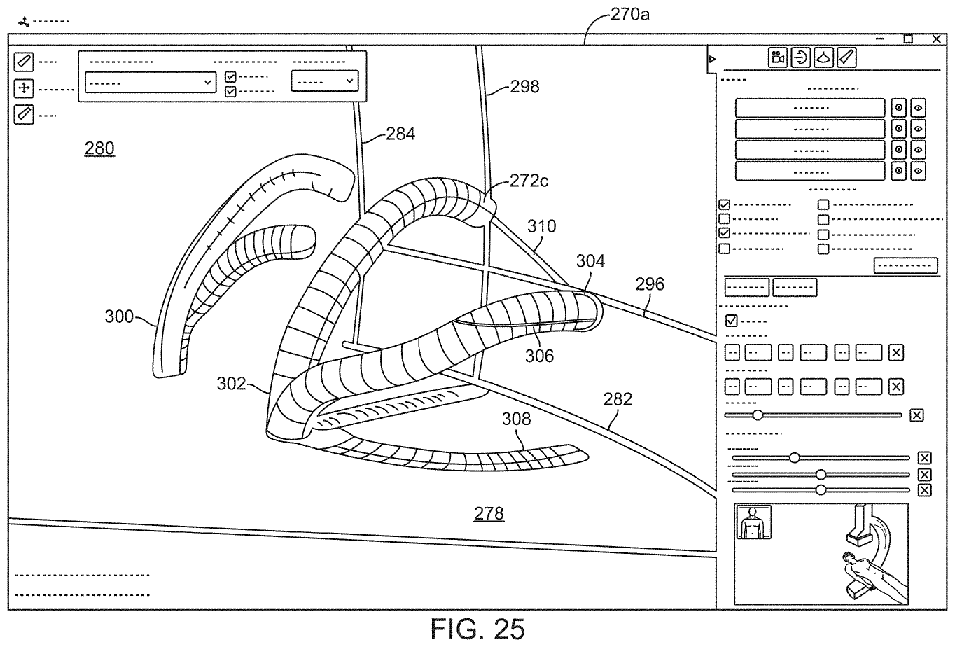

Referring now to FIG. 25, hybrid images may advantageously present combinations of acquired images of an actual catheter 300, images of a virtual or model catheter 302, images of phantom or candidate catheter poses 304, or all three. The acquired images will often be presented as 2D images on associated image planes; the phantom and virtual or model catheter images optionally being presented as 3D model images or 2D images superimposed on the acquired images or both. When 3D model image (for example, 3D phantom image 304) will be presented together with ultrasound image slices (such as along first echo plane 296) it can be beneficial to highlight a model/slice intersection 306 of the 3D model, and to superimpose a 2D intersection image 308 of the highlighted intersection on the acquired echo image plane 278. As can be understood with reference to FIGS. 16A-16D, 19, 25, and the associated text, phantom or candidate pose virtual models of the catheters or other articulated elongate bodies can be based initially on a calculated pose of the catheter that is virtually modified by entering movement commands using an input system 204. The modified pose can be determined using a simulation module 256 of processor 214. The system can display trajectory 310 between the model pose and the candidate pose in 3D with the 3D models and the user can input commands to the input system to advance along the trajectory, stop, or retreat along the trajectory as described above.

Referring to FIGS. 19 and 22-25, in the lower right portion of the hybrid display is a graphical reference frame indicator showing offset orientations of the display frame and at least one imaging system frame. The reference frame indicator optionally presents overall tissue position and an associated reference frame 246 graphically, preferably by including an image of some or all of the patient body (such as the body, torso, heart, or head) indicative of a position and orientation of tissues targeted for treatment or diagnosis. An orientation of an image capture device and associated reference frame 240, 242 relative to the patient body is also presented graphically with an image capture orientation indicator, preferably using an image of the associated imaging device (such as an image of a C-arm, TEE probe, ICE probe, or the like). An orientation of the display reference frame 208 relative to the patient body is presented graphically using, for example, a schematic image of a camera, a reference frame, or the like.

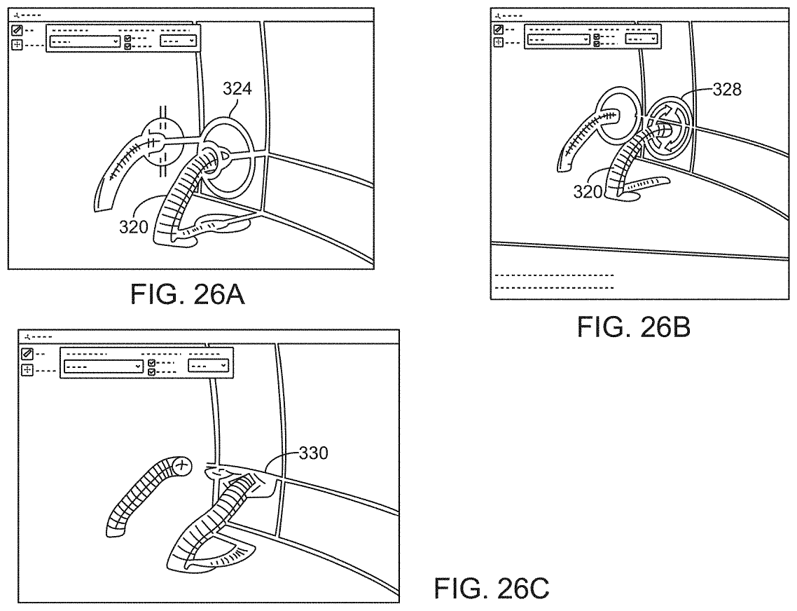

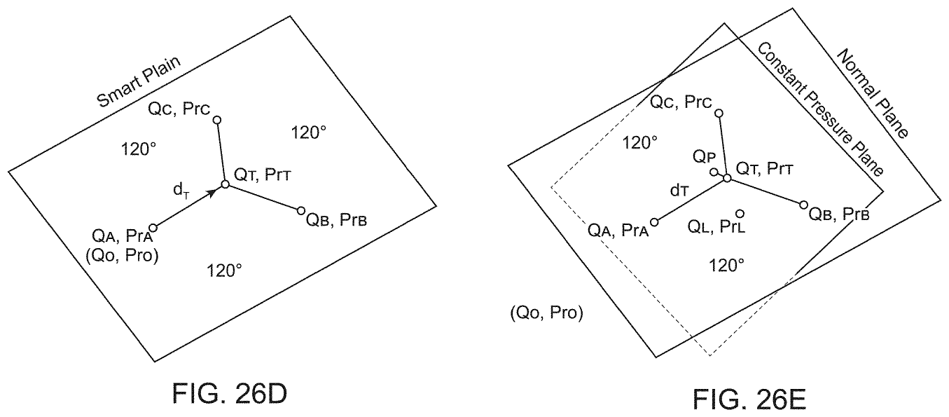

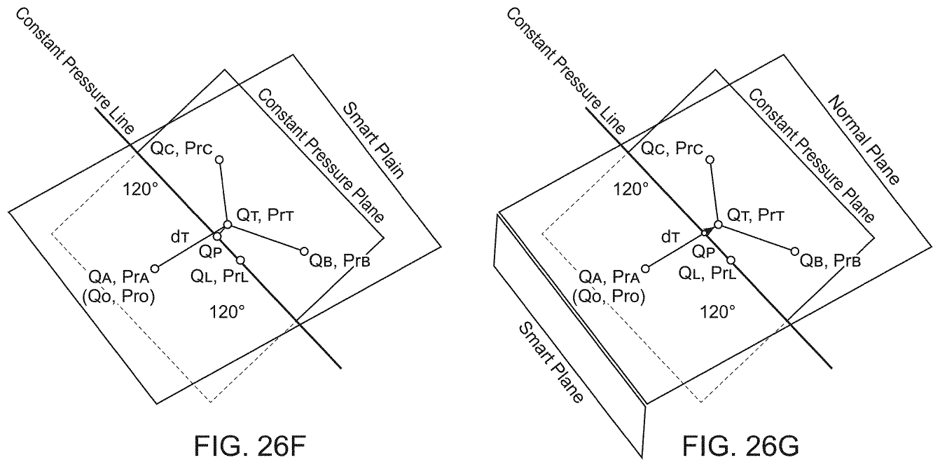

Referring now to FIGS. 1, 19 and 26A-26C, processor 214 can optionally provide a plurality of alternative constrained motion modes, and may superimpose indicia of movement available in those constrained modes in the auxiliary display image 252. For example, in FIG. 26A 3D virtual model image 320 in an unconstrained movement mode allows the user to input movement commands 212 using a 6 DOF input device 16. To graphically indicate to the user that movement commands in the display space 208 can induce translation of the catheter, rotation of the catheter, or combinations of both, an unconstrained movement widget 324 is superimposed on the tip of the 3D catheter model image in the auxiliary display image 252, with the unconstrained movement widget including a semi-transparent sphere indicative of allowability of rotation commands and an extended catheter axis indicative of allowability of translation commands. Optionally, corresponding 2D widgets may similarly be projected to one, some, or all of the 2D image planes and superimposed on the 2D virtual and/or captured catheter images.

Referring now to FIG. 26B, an advantageous plane-and-spin constrained mode allows the user to identify a plane, and then allows input commands to induce constrained movement of the catheter tip to the extent that the input commands induce: i) translation of the tool, tip, or receptacle along the identified plane, and/or ii) rotation of the tool, tip, or receptacle about an axis normal to the identified plane. The constraining of the input commands for this plane-and-spin mode (and other constrained movement modes of the system) are performed by a constraints module of processor 214, and a plane-and-spin widget indicative of this mode superimposed on the catheter image may comprise a disk centered on the tip or receptacle and parallel to the selected constraint plane, along with rotational arrows parallel to the disk. This plane-and-spin constrained movement mode is particularly advantageous for use with planar image acquisition systems as the user can select a 2D image plane. When movement commands are entered with the system in this mode using, for example, a 6 DOF input device 16, only the components of the input commands along the plane or rotational about the normal will induce movement of the catheter, so that the tip of the catheter can remain at a constant plane offset (the offset often being negligible so that the tip remains along the plane) and angular orientation relative to the plane (sometimes referred to herein as the same pitch relative to the plane), so that the user looking at a 2D image along that plane has good optical cues on which to evaluate movement and alignment with tissues that can be seen in that 2D image.

Referring now to FIG. 26C, a normal-and-pitch constrained movement mode (and associated normal-and-pitch constraint indicia 330) is largely complimentary to the plane-and-spin constrained mode described above. More specifically, when in this mode input commands are only considered to the extent that they are normal to the selected plane, and/or to the extent that the input commands seek changes in pitch, i.e., rotation about an axis parallel to the selected plane and perpendicular to the axis of the tip. When a user enters this mode catheter movement is inhibited in an acquired image along the selected plane (the movement being limited to movement into and out of the image plane, and/or to changes in pitch) so that if this mode is selected when the 2D image of the catheter is aligned with the 2D image of a target tissue the catheter will primarily remained aligned with that target tissue, somewhat giving the impression that the catheter is locked in alignment for the selected plane. The user will generally make use of this mode while viewing a 2D image at an angularly offset orientation from the selected plane or while viewing a 3D image of the catheter. The exemplary normal-and-pitch constrained indicia comprises an axis along the allowed motion normal to the selected plane, and rotational arrows about the pitch axis, with these pitch arrows being disposed on a cylindrical surface to help differentiate the spin arrow.

Regarding the functionality and data processing for which constraints module 326 is configured, the following section provides additional details.

Boundary and Constraint Control Mode Types & Function

Five distinct control modes of processor 214 are described herein for addressing workspace boundaries and other constraints and which may optionally be implemented in constraints module 326 (see FIG. 19). Input module 216 and simulation module 256 will often be used to generate input command signals for pressure control module 230, and constraints module 326 will optionally use the functionality described below to return telemetry back to the simulation and/or input command modules when the pressure control module determines that the commanded movement may encounter a workspace boundary, typically in the form of a pressure boundary when using the preferred articulation balloon array structures described above. While these control modes are often described below with reference to pressure limits and associated calculations, alternative embodiments may make use of torque limits or the like while similarly constraining motions to planes, lines, and the like. Similarly, while the exemplary simulation module 256 is implemented using machine-readable code in a Unity™ 3D graphical software environment (so that the description below may, for example, reference communications between the pressure controller and Unity™), it should be understood that alternative environments may be implemented using communications between the pressure control module 256 (sometimes referred to below as the Controller) and an alternative commercially available or proprietary 3D graphical software engine. The simulation module optionally uses spatial modes that are somewhat similar to those described below for controlling the catheter tip or tool receptacle of the therapy delivery system, but may not alter telemetry output from the pressure module back to the simulation (sometimes referenced below as telemetry) and so that processor 214 may benefit from the alternative constraint functionality described below to help pressure controller module 230 to meet the boundary limitations with appropriate telemetry back to the simulation module 256. The constraint control modes described herein include:
5D Shift/Scale Mode
3D Gradient Mode
Planar Mode
Line Mode
Gimbal Mode
Axis Mode
Segment Mode

Mode Table: Table 2 below describes the purpose and the general interaction between the input system 204, simulation module 256, and the pressure controller 230 (and particularly the response telemetry from the pressure controller to the simulation module).

TABLE 2

| Control | Driven | Adjusted DOF | Fixed |
|---|---|---|---|
| Mode | DOF | (at boundary) | DOF |
| 5D | All | 3D Position | None |
| Shift/Scale | | first, then | |
| | | Orientation | |
| 3D | All | 3D | None |
| Gradient | | Position | |
| Plane | Planar Position, | Planar | Normal |
| | 2D | Position | Position |
| | Rotation | | |
| Line | Normal Position, | Lineal | Planar |
| | 2D | Position | Position |
| | Rotation | | |
| Gimbal | 2D | 2D | 3D |
| | Rotation | Rotation | Position |
| Axial | 1D | None | 3D Position |
| | Rotation | | 1D Rotation |
| | (Driven) | | (Passive) |
| Segment | Drive Segment | Passive | Dependent |
| | (+ other | Segments | on other |
| | modes) | (+ other modes) | modes |

5D Shift/Scale vs. 3D Gradient

This is for motion unconstrained (other than pressure limits) in 5/6D space. The planar position can give way to achieve the orientation commanded. It is used with an unconstrained 6DOF input such as a Tango™ smartphone in free space and at pressure boundaries. There are two modalities described herein:

Shift/Scale Mode:

This uses the Shift and Scale functions to respond to the pressure boundary; and

Gradient Mode:

This function uses three points (A, B, C) with set orientations and in the vicinity of the goal Q (as defined by the input) to form a local linear 3D pressure gradient to estimate the goal position or the closest achievable position in the 3D space.

Planar Mode:

This is for motion constrained to a plane. The planar position can give way to achieve the orientation commanded. It is used when, for example, the Tango™ 6DOF input system (sometimes referenced below as Tango™) constrains motion to a plane, when the mouse can be used for translation commands (optionally when the left button is held) to move on a plane, and optionally when the roller button is held and the mouse moves on a plane for changing orientation. This mode uses the three point (A-B-C) Planar function. (Note that the Roll function optionally utilizes Line mode).

Line Mode:

This is for motion constrained to a Line. The line position can give way to achieve the orientation commanded. It can be used when Tango constrains motion to a line and optionally with the mouse Roll function.

Gimbal Mode:

This is for motion constrained to a point in space. The point does not give way. Tip orientation will slide along the orientation boundary and find the closest Tip position and telemetry. This mode can be used when Tango™ constrains motion to a point and optionally when the mouse roller button is held for orientation control.

Axial Mode:

This is for motion constrained to a point with rotating fixed to one axis in space. The point and axis do not give way, and the single driven orientation may be achievable while the tip remains on this point. The tip stops at the workspace (pressure) boundary. It can be used when simulation module 256 constrains motion to a point and a single axis.

Segment Mode:

This is for driving motion on individual segments at segment transitions where one segment is driven to articulate and elongate. The passive segments respond in a manor preset by the user. For example, the passive segments may be set to hold their orientation and position relative to its own segment base. A second example is that passive segments may be set to stay on a point, trajectory, or plane. This mode can be driven by Tango™, a mouse, and other input forms. In this mode different segments may be set to behave with or without spatial constraints utilizing some of the properties in the previously listed Modes.

Simulation module 256 sends to the pressure control module 230 the input mode, input parameters, and the trajectory point(s). The input data is different for different modes as follows. Note that the Target Data and Command Data sets will often both utilize this input mode strategy.

Input Data
Shift/Scale—Mode, Parameters, Q_{Command}, Q_{Target}
3D Gradient—Mode, Parameters, Q_{A}, Q_{B}, Q_{C} (for both command and target set)
Planar Mode—Mode, Parameters, Q_{A}, Q_{B}, Q_{C} (for both command and target set)
Line Mode—Mode, Parameters, Q_{A}, Q_{B}, Q_{C} (for both command and target set)
Gimbal Mode—Mode, Parameters, Q_{A}, Q_{B}, Q_{C} (for both command and target set)
Axial Mode—Mode, Parameters, Q_{A}, Q_{B}, Q_{C} (for both command and target set)
Segment Mode—Mode, Parameters, TBD, but either Q (world space), j (joint space), or a combination

The pressure control module functions differently in each mode.

Pressure Control Module Function
Shift/Scale—Iterates 3 times for best solution, uses Shift and Scale as needed.
3D Gradient—Iterates 3 times, but only once on each of Q_{A}, Q_{B}, Q_{C} and then creates lumen pressure gradient to find Q_{T} or Q_{P} (closest achievable point in 3D space)
Planar Mode—Iterates 3 times, but only once on each of Q_{A}, Q_{B}, Q_{C} and then creates lumen pressure gradient to find Q_{T} or Q_{P} (closest achievable point on the Plane)
Line Mode—Iterates 3 times, but only once on each of Q_{A}, O_{B}, Q_{C} and then creates lumen pressure gradient to find O_{T} or Op (closest achievable point on the Line)
Gimbal Mode—Iterates 3 times, but only once on each of Q_{A}, O_{B}, Q_{C} and then creates lumen pressure gradient to find O_{T} or Op (closest achievable orientation while maintaining point location)
Axial Mode—Iterates 3 times, but only once on each of Q_{A}, O_{B}, Q_{C} and then creates lumen pressure gradient to find O_{T} or Op (closest achievable driven angle while maintaining passive angle)

The pressure control module 230 sends simulation module 256 the error conditions, boundary conditions and trajectory data for the command, phantom, and actual segments.

Trajectory Data
5D Shift/Scale—error, boundary, j_{Command}, j_{Phantom}, j_{Actual}
3D Gradient—error, boundary, j_{Command}, j_{Phantom}, j_{Actual}
Planar Mode—error, boundary, j_{Command}, j_{Phantom}, j_{Actual}
Line Mode—error, boundary, j_{Command}, j_{Phantom}, j_{Actual}
Gimbal Mode—error, boundary, j_{Command}, j_{Phantom}, j_{Actual}
Axial Mode—error, boundary, j_{Command}, j_{Phantom}, j_{Actual}
Segment Mode—error, boundary, j_{Command}, j_{Phantom}, j_{Actual}

Simulation module 256 uses the error conditions, boundary conditions, and trajectory data to proceed with the next action.

Setting Up the Pressure Gradient with Fixed Orientation

Scaling & Shifting Function Limitations

The pressure control module 230 optionally uses the kinematic equations to find a solution for the goal QT. The solution produces a pressure vector, Pr_{T}, based on the Jacobean of the current location. This solution does not account for workspace boundaries in the form of lumen pressure limits. When the Pr_{T} vector includes components outside the maximum and minimum pressure limits, two functions may be implemented to find the closest achievable solution. The first is to shift segment-based pressures values into range. Shifting maintains orientation at the sacrifice of position. When a segments lumen pressure range is too large to shift, a secondary function scales the individual segment-based pressures. The scaling changes both the position and the orientation from the goal QT. The result of Shifting and Scaling is that the telemetry produced tends towards the closest special position available sometimes maintaining and other times changing the tip orientation. This Shifting and Scaling can be functional while the Goal Q_{T} is only constrained by a pressure boundary, though it may not produce the goal orientation. Scaling may (at least in some cases) inherently change the segments' orientation.

Gradient Control

Gradient control is a method for finding the closest positional telemetry at engaging the boundary, with or without additional spatial constraints, while maintaining the goal orientation. The function finds the closest available position Q_{d} to the goal Q_{T}. This method is an alternative for the Shifting and Scaling functions for finding the Q trajectories. Either methods may to be utilized in the pressure control module code at different times; Shifting and Scaling for unconstrained orientation, and the Gradient Control for when maintaining goal orientation with (or without) spatial constraints is preferred.

General Model and Steps Towards Finding the Closest Gradient Solution

With reference to FIG. 26D, points T, A, B, and C are located on a plane that intersects the goal position Q_{T}. Optionally, it might also intersect a line formed between a start position and a goal position Q_{T}. The orientation of the Plane depends on the spatial constraints. With no spatial constraints or when constrained to a line, the plane resides on the trajectory line and the orientation is arbitrary, though there may be preferable orientations and it may be desirable to vary the orientation in subsequent cycles. When constrained to a plane (such as Smart Plane) the orientation is defined. Each point references unique position coordinates of associated Q vectors. The Q orientation values (alpha and beta) for each Q vector are the same which allows for three unique positions (X, Y, Z) at three different pressure values (per lumen). Simulation module 256 derives Q_{A}, Q_{B}, and Q_{C} tip vectors based the desired Q_{T} input, and sends the three Q vectors to the pressure control module. The pressure control module solves for each lumen pressure (Pr_{A}, P_{B}, Pr_{C}) for each Q position. A lumen pressure gradient between Q positions is generated. The lumen pressure vector at the target point (Pr_{T}) is solved. If Pr_{T} has one or more lumen pressures outside the lumen pressure limits, the gradient equations are used to find the closest alternative Q that is within pressure limits and maintains orientation.

For displacement commands, the pressure control module allows the user to slide the Tip along the boundary and to achieve the closest solution. For pivoting commands, displacement occurs only when at a boundary. For both, the limit of travel is to the closest position while achieving the goal orientation by shifting on the along the boundary.

Symbols
Q_{O}—current tip position in world space.
Q_{T}—goal position (Tip input) in world space.
Q_{A}, Q_{B}, Q_{C}—on Plane; equal distance to Q_{T}; 120° offset from each other around Q_{T}; Optionally Q_{A} is on line formed from Q_{O} and Q_{T}.
\vec{Pr}—vector containing each lumen pressure at a specific Q position.
d_{T}—is the planar target offset.

Simulation Module/Pressure Control Module Communication

Simulation module 256 solves for the planar Q's (Q_{A}, Q_{B}, Q_{C}) based on Q_{T} and sends the three Q vectors to the pressure control module 230. The pressure control module solves for the three pressure vectors (Pr_{A}, P_{B}, Pr_{C}), produces the pressure gradients, solves for the lumen pressures Pr_{T} or Pr_{P}, and sends position telemetry Q_{T} or Q_{P} to the simulation module.

Gradient Model

The following Gradient Math occurs in the pressure control module after receiving the Q vectors from the simulation module. This gradient model applies to the 3D Gradient Mode, Planar Mode, and the Line Mode.

Spatial Plane

Find the plane formed by the positions of Q_{A}, Q_{B}, and Q_{C}, using the X, Y, & Z component.

C_{X0}*(X−X_{A})+C_{Y0}*(Y−Y_{A})+C_{Z0}*(Z−Z_{A})=0

(1)
C_{X0}, C_{Y0}, & C_{X0} are the plane constants associated with A, B, and C points. A vector formed by (C_{X0}, C_{Y0}, C_{Z0}) is perpendicular to plane.
X_{A}, Y_{A}, Z_{A} are known position points of Q_{A} and can be any known point.
X, Y, & Z are the Q position coordinate variables.

Find the plane constants using the cross-product, to find the perpendicular vector.

V₁=B−A=[(X_{B}−X_{A}), (Y_{B}−Y_{A}), (Z_{B}−Z_{A})]

V₂=C−A=[(X_{C}−X_{A}),(Y_{C}−Y_{A}),(Z_{C}−Z_{A})]

V_{P0}=V₁×V₂=(C_{X0},C_{Y0},C_{Z0})

C_{X0}=(Y_{B}−Y_{A})*(Z_{C}−Z_{A})−(Y_{C}−Y_{A})*(Z_{B}−Z_{A})

C_{Y0}=(X_{C}−X_{A})*(Z_{B}−Z_{A})−(X_{B}−X_{A})*(Z_{C}−Z_{A})

C_{Z0}=(X_{B}−X_{A})*(Y_{C}−Y_{A})−(X_{C}−X_{A})*(Y_{B}−Y_{A})

From equation 1

C_{X0}*X+C_{Y0}*Y+C_{Z0}*Z=C_{X0}*X_{A}+C_{Y0}*Y_{A}+C_{Z0}*Z_{A}

P_{L0}=C_{X0}*X_{A}+C_{Y0}*Y_{A}+C_{Z0}*Z_{A} P_{L0} is the ABC plain constant

Pressure Plane

We assume a linear gradient for the lumen pressure variance throughout the Q sample range. Use the following formulas to solve for the planer “C” constants.

C_{Xi}*X+C_{Yi}*Y+C_{Zi}*Z=Prᵢ

[2]
“i” represents the lumen number; for a two-segment system “i” is from 1 through 6.
C_{Xi}, C_{Yi}, & C_{Xi} are the pressure constants to estimate the pressure of lumen “i”. A vector formed by (C_{Xi}, C_{Yi}, C_{Zi}) is perpendicular to the “i” lumen pressure plane.
X, Y, & Z are the Q position coordinate variables.
Prᵢ is the estimated pressure of lumen “i” at position X, Y, and Z.

For each of the three Q's defined by A, B, and C, a position (X, Y, Z) and Lumen Pressure (Prᵢ) are known. Set up equation and solve for constants for each lumen pressure.

C_{Xi}*X_{A}+C_{Yi}*Y_{A}+C_{Zi}*Z_{A}=Pr_{Ai}

C_{Xi}*X_{B}+C_{Yi}*Y_{B}+C_{Zi}*Z_{B}=Pr_{Bi}

C_{Xi}*X_{C}±C_{Yi}*Y_{C}+C_{Zi}*Z_{C}=Pr_{Ci}

$$[ XYZ ] \cdot {\vec{C}}_{i} = {\vec{P r}}_{i} \\ [ XYZ ] = ( \begin{matrix}{X}_{A} & {Y}_{A} & {Z}_{A} \\ {X}_{B} & {Y}_{B} & {Z}_{B} \\ {X}_{C} & {Y}_{C} & {Z}_{C}\end{matrix} )$$

$${\vec{C}}_{i} = {[ XYZ ]}^{- 1} \cdot {\vec{P r}}_{i}$$

Including additional Q points which can be retained from the previous cycle, an alternative may be implemented.

\vec{C}ᵢ=(XYZ·XYZ^{T})⁻¹·XYZ^{T}·\vec{Pr}ᵢ

Goal Position

Geometrically, Q_{T} is the average of Q_{A}, Q_{B}, and Q_{C} and expressed as follows:

$${\vec{Q}}_{T} = ( {\vec{Q}}_{A} + {\vec{Q}}_{B} + \vec{Q} c \text{)/3} \\ \begin{matrix}{X}_{T} = ( {X}_{A} + {X}_{B} + {X}_{C} \text{)/3} ; & {Y}_{T} = ( {Y}_{A} + {Y}_{B} + {Y}_{C} \text{)/3} ; & \end{matrix} {Z}_{T} = ( {Z}_{A} + {Z}_{B} + {Z}_{C} \text{)/3}$$

Goal Lumen Pressure

The pressure vector can be found through the “C” constants vector:

\vec{Pr}_{I}=\vec{C}_{I}·\vec{Q}_{T}

Use equation 2 (6 times for two segments) and solve for the target lumen pressures \vec{Pr}_{T} (at position Q_{T}).

If all lumen pressures at \vec{Pr}_{T} are within the pressure limits, use the current pressure vector. If one or more pressure components are outside the limits, solve for the closest position on the Smart Plane where all pressures are within the pressure limits.

3D Gradient Mode

Pressure Limit Points

Referring now to FIG. 26E, for lumens that are not within a pressure limit, find the point on the limit plane when intersected by a normal line also intersects the goal point at Q_{T}. If there are more than one lumen outside the pressure limit, and the all these normal limit points produce pressure vectors with a component outside the limit, then find the closest pressure limit point at the intersection of paired planes and search these for the closest achievable lumen pressure vector.

Goal Point Normal Line

The Vector normal to the pressure plane is a derivative of the plane definition.

\vec{V}_{Ni}=(V_{NXi},V_{NYi},V_{Nzi})=(C_{Xi},C_{Yi},C_{Zi})

This vector may be normalized to make it a unit vector.

Normal Line Constants, Normal Line Equations, and Intersect Point Q_{di} (at Pressure Limit),

Choose best axis by choosing largest absolute value of \vec{V}_{Ni} components, (C_{Xi}, C_{Yi}, C_{Zi}). If |C_{Xi}| is the largest, solve for Normal Line Constants with X as variable. If |C_{Yi}| is the largest, solve for Normal Line Constants with Y as variable. If |C_{Zi}| is the largest, solve for Normal Line Constants with Z as variable.

TABLE 3

Variable Axis

| | X | Y | Z |
|---|---|---|---|
| ΔN_{Xi} | | V_{PXik}/V_{PYik} | V_{PXik}/V_{PZik} |
| N_{Xi} | | X_{Pi} − (V_{PXik}/ | X_{Pi} − (V_{PXik}/ |
| | | V_{PYik})*Y_{Pi} | V_{PZik})*Z_{Pi} |
| ΔN_{Yi} | C_{Yi}/C_{Xi} | | V_{PYik}/V_{PZik} |
| N_{Yi} | Y_{T} − (C_{Yi}/C_{Xi})*X_{T} | | Y_{Pi} − (V_{PYik}/ |
| | | | V_{PZik})*Z_{Pi} |
| ΔN_{Zi} | C_{Zi}/C_{Xi} | V_{PZik}/V_{PYik} | |
| N_{Zi} | Z_{T} − (C_{Zi}/C_{Xi})*X_{T} | Z_{Pi} − (V_{PYik}/ | |
| | | V_{PXik})*Y_{Pi} | |

Define the Intersection Line of each pressure limit plane. Use the same variable axis as with the TABLE 3 above. Line equations are formed from the equations of TABLE 4 below.

TABLE 4

Variable Axis

| | X | Y | Z |
|---|---|---|---|
| X_{Pi} | X_{Pi} | N_{Xi} + ΔN_{Xi}*X_{Pi} | N_{Xi} + ΔN_{Xi}*Z_{Pi} |
| Y_{Pi} | N_{Yi} + ΔN_{Yi}*X_{Pi} | Y_{Pi} | N_{Yi} + ΔN_{Yi}*Z_{Pi} |
| Z_{Pi} | N_{ZXi} + ΔN_{ZXi}*X_{Pi} | N_{Zi} + ΔN_{Zi}*X_{Pi} | Z_{Pi} |

Find the intersection point of the Normal line and the constant pressure plane, for each lumen that is not within the pressure limits.

Insert “X” line equations:

C_{Xi}*X_{Pi}+C_{Yi}*Y_{Pi}+C_{Zi}*Z_{Pi}=Pr_{Limit}

[from 2]

C_{Xi}*X_{Pi}+C_{Yi}*(N_{Yi}+N_{YXi}*X_{Pi})+C_{Zi}*(N_{Zi}+N_{ZXi}*X_{Pi})=Prᵢ

C_{Xi}*X_{Pi}+C_{Yi}*N_{Yi}+C_{Yi}*N_{YXi}*X_{Pi}+C_{Zi}*N_{Zi}+C_{Zi}*N_{ZXi}*X_{Pi}=Prᵢ

(C_{Xi}+C_{Yi}*N_{YXi}+C_{Zi}*N_{ZXi})*X_{Pi}+(C_{Yi}*N_{Yi}+C_{Zi}*N_{Zi})=Prᵢ

X_{Pi}=[Pr_{Limit}−(C_{Yi}*N_{Yi}+C_{Zi}*N_{Zi})]/(C_{Xi}+C_{Yi}*N_{YXi}+C_{Zi}*N_{ZXi})

Y_{Pi}=N_{Yi}+N_{YXi}*X_{Pi}

Z_{Pi}=N_{Zi}+N_{ZXi}*X_{Pi}

“Y” and “Z” line equations are similarly resolved.

TABLE 5

Variable Axis

| | X | Y | Z |
|---|---|---|---|
| X_{Pi} | [Pr_{Limit} − | X_{Pi} = N_{Xi} + | N_{Xi} + ΔN_{Xi}*Z_{Pi} |
| | (C_{Yi}*N_{Yi} + | ΔN_{Xi}*Y_{Pi} | |
| | C_{Zi}*N_{Zi})]/(C_{Xi} + | | |
| | C_{Yi}*ΔN_{Yi} + | | |
| | C_{Zi}*ΔN_{Zi}) | | |
| Y_{Pi} | N_{Yi} + ΔN_{Yi}*X_{Pi} | [Pr_{Limit} − | N_{Yi} + ΔN_{Yi}*Z_{Pi} |
| | | (C_{Xi}*N_{Xi} + | |
| | | C_{Zi}*N_{Zi})]/ | |
| | | (C_{Xi}*ΔN_{Xi} + | |
| | | C_{Yi} + C_{Zi}*ΔN_{Zi}) | |
| Z_{Pi} | N_{Zi} + ΔN_{Zi}*X_{P} | N_{Zi} + ΔN_{Zi}*X_{Pi} | [Pr_{Limit} − |
| | | | (C_{Xi}*N_{Xi} + |
| | | | C_{Yi}*N_{Yi})]/ |
| | | | (C_{Xi}*ΔN_{Xi} + |
| | | | C_{Yi}*ΔN_{Yi} + C_{Zi}) |

Plane Crossing Vector

\vec{V}_{Cik}=\vec{V}_{Ni}×\vec{V}_{Nk}=(V_{CXi},V_{CYi},V_{CZi})

(V_{CXik},V_{CYik},V_{CZik})=[(V_{NYi}*V_{NZk}−V_{NZi}*V_{NYk}),(V_{NZi}*V_{NXk}−V_{NXi}*V_{NZk}),(V_{NXi}*V_{NYk}−V_{NYi}*V_{NXk})]

This vector may be normalized to make it a unit vector.

Perpendicular Vector from Normal Point to Plane Crossing Vector

\vec{V}_{Pik}=\vec{V}_{Ni}×\vec{V}_{Cik}=(V_{PXi},V_{PYi},V_{PZi})

(V_{PXik},V_{PYik},V_{PZik})=[(V_{NYi}*V_{CZik}−V_{NZi}*V_{CYik}),(V_{NZi}*V_{CXik}−V_{NXi}*V_{CZik}),(V_{NXi}*V_{CYik}−V_{NYi}*V_{CXik})]

Perpendicular Line Constants, Equations, and Intersect Point Pik (at Pressure Limit),

Choose best axis by associated with the smallest absolute value of \vec{V}_{Pik} components, (V_{PXik}, V_{PYik}, V_{PZik}).

If |V_{PXik}| is the smallest, solve for perpendicular Line Constants with X as variable. If |V_{PYik}| is the smallest, solve for perpendicular Line Constants with Y as variable. If |V_{PZik}| is the smallest, solve for perpendicular Line Constants with Y as variable.

TABLE 6

Variable Axis

| | X | Y | Z |
|---|---|---|---|
| ΔK_{Xik} | | V_{PXik}/V_{PYik} | V_{PXik}/V_{PZik} |
| K_{Xik} | | X_{Pi} − (V_{PXik}/ | X_{Pi} − (V_{PXik}/ |
| | | V_{PYik})*Y_{Pi} | V_{PZik})*Z_{Pi} |
| ΔK_{Yik} | V_{PYik}/V_{PXik} | | V_{PYik}/V_{PZik} |
| K_{Yik} | Y_{Pi} − (V_{PYik}/ | | Y_{Pi} − (V_{PYik}/ |
| | V_{PXik})*X_{Pi} | | V_{PZik})*Z_{Pi} |
| ΔK_{Zik} | V_{PZik}/V_{PXik} | V_{PZik}/V_{PYik} | |
| K_{Zik} | Z_{Pi} − (V_{PZik}/ | Z_{Pi} − (V_{PYik}/ | |
| | V_{PXik})*W_{Pi} | V_{PXik})*Y_{Pi} | |

Find Line Intersection Point (X_{P}, Y_{P}, Z_{P}) on each plane intersection line. Use the same variable axis as with the Perpendicular Line Constants. Line equations can be formed from the following equivalencies.

“X” as variable input

K_{ZXik}+ΔK_{ZXik}*X_{Pi}=K_{Zki}+ΔK_{ZXki}*X_{Pi} or K_{YXik}+ΔK_{YXik}*X_{Pi}=K_{YXki}+ΔK_{YXki}*X_{Pi}

“Y” as variable input

K_{ZYik}+ΔK_{ZYik}*Y_{Pi}=K_{ZYki}+ΔK_{ZYki}*Y_{Pi} or K_{XYik}+ΔK_{XYik}*Y_{Pi}=K_{XYki}+ΔK_{XYki}*Y_{Pi}

“Z” as variable input

K_{YZik}+ΔK_{YZik}*Z_{Pi}=K_{Yki}+ΔK_{YZki}*Z_{Pi} or K_{Xik}+K_{XZik}*Z_{Pi}=K_{Xki}+K_{XZki}*Z_{Pi}

TABLE 7

| | Variable Axis | | |
|---|---|---|---|
| | X | Y | Z |
| X_{Pik} | (K_{Zki} − K_{Zik})/ | K_{Xik} + ΔK_{Xik}*Y_{P} | K_{Xik} + ΔK_{Xik}*Z_{P} |
| | (ΔK_{Zik} − ΔK_{Zki}) | | |
| Y_{Pik} | K_{Yik} + ΔK_{Yik}*X_{Pik} | (K_{Zki} − K_{Zik})/ | K_{Yik} + ΔK_{Yik}*Z_{P} |
| | | (ΔK_{Zik} − ΔK_{Zki}) | |
| Z_{Pik} | K_{Zik} + ΔK_{Zik}*X_{Pik} | K_{Zik} + ΔK_{Zik}*Y_{P} | (K_{Yki} − K_{Yik})/ |
| | | | (ΔK_{Yik} − ΔK_{Yki}) |

Solve the pressure array(s) for each plane intersection line Point

\vec{Pr}_{Ti}=\vec{C}ᵢ*(X_{Pi},Y_{Pi},Z_{Pi})

\vec{Pr}_{Tik}=\vec{C}ᵢ*(X_{Pik},Y_{Pik},Z_{Pik})

Solve for the intersect points on the line formed (by lumens beyond the pressure limit) from crossing limit planes. The number of points may be dictated by the number of lumens that cross a limit pressure. The max number of planes for two segments may be six.

In the Table 8 below the i and k indicate a specific lumen combination where i and k are not be the same number and all combinations can only be selected once.

TABLE 8
i

| | | 1 | 2 | 3 | 4 | 5 | 6 |
|---|---|---|---|---|---|---|---|
| | 1 | | x | x | x | x | x |
| | 2 | | | x | x | x | x |
| k | 3 | | | | x | x | x |
| | 4 | | | | | x | x |
| | 5 | | | | | | x |
| | 6 | | | | | | |

This chart indicates the number of lumen plane intersect points as a function of the number of lumen lines in play. Note that with six lumen planes there are 15 intersect lines and associated points as indicated by the “x's”. Add this to the Normal line intersects, with six lumens over the pressure limit and a total of 21 intersect points (6 normal+15 plane line points) may benefit from being resolved.

Now find the closest (and achievable) point Q_{P} to the target Q_{T} (with all pressure values within the limits). There should be one intersect point with the Pr vector within the pressure limits. To optimize the search sequence, note the following:
An achievable normal intersection points will be closer than any achievable plane or line intersection points. The Normal intersection point comes from the normal line through the goal point.
The farthest Normal intersection point will generally be the closest Normal point that can be achieved (within the pressure limits). If it is not within the pressure limits, the closest point will be one of the lumens' intersect plane line points.
It may be possible, for a lumen which is not pressure limited across Q_{A}, Q_{B}, or Q_{C} to be an intersect limit line that defines the closest point.

Planar Mode

Pressure Limit Points

Referring now to FIG. 26F, for lumens that are not within a pressure limit, find the points on lines A-B, B-C, and C-A that cross the limit pressure line. These points will all be in a line in space.

First, determine if pressure limit plane is normal to the A-B-C plane, in which case the pressure may be parallel to the allowable direction of motion and there may be no solution. For this condition return the previous telemetry.

Second, determine which limit points are farthest apart. This avoids using two points with the same pressure which leads to a non-zero divisor when finding the points.

ΔPr_{ABC}=Pr_{AB}−Pr_{BC}

ΔPr_{BCA}=Pr_{BC}−Pr_{CA}

ΔPr_{CAB}=Pr_{CA}−Pr_{AB}

MinΔPr=0.001—Other numbers may be determined empirically from trial and error or derived.
Flag=IF(MAX(ΔPr_{ABC},ΔPr_{BCA},ΔPr_{CAB})<MinΔPr,“Normal”,
   IF(AND(ABS(ΔPr_{ABC})>=ABS(ΔPr_{BCA}),(ΔPr_{ABC})>=ABS(ΔPr_{CAB}),“AB”,
   IF(ΔPr_{BCA}>=ABS(ΔPr_{CAB}),“BC”,
   “CA”)))
=IF(Flag=“Normal”,<return previous telemetry>,
   IF(Flag=“AB”,

(X_{Di},Y_{Di},Z_{Di})=(Pr_{Limit}−Pr_{Ai})/(Pr_{Bi}−Pr_{Ai})*[(X_{B},Y_{B},Z_{B})−(X_{A},Y_{A},Z_{A})]+(X_{A},Y_{A},Z_{A}),
IF(Flag=“BC”,

(X_{Di},Y_{Di},Z_{Di})=(Pr_{Limit}=Pr_{Bi})/(Pr_{Ci}−Pr_{Bi})*[(X_{C},Y_{C},Z_{C})−(X_{B},Y_{B},Z_{B})]+(X_{B},Y_{B},Z_{B}),
IF(Flag=“CE”,

(X_{Di},Y_{Di},Z_{Di})=(Pr_{Limit}−Pr_{Ci})/(Pr_{Ai}−Pr_{Ci})*[(X_{A},Y_{A},Z_{A})−(X_{C},Y_{C},Z_{C})]+(X_{C},Y_{C},Z_{C}),
<missing flag>))))

Limit Line Vector

Find the pressure Limit Line (unit) Vector with the cross product of the pressure and A-B-C plane's normal vectors, which can be taken directly from the plane constants above.

\vec{V}_{L}=(C_{Xi},C_{Yi},C_{Zi})×(C_{X0},C_{Y0},C_{Z0})=(V_{LXi},V_{LYi},V_{LZi})

(V_{LXi},V_{LYi},V_{LZi})=[(C_{Yi}*C_{Z0}−C_{Zi}*C_{Y0}),(C_{Zi}*C_{X0}−C_{Xi}*C_{Z0}),(C_{Xi}*C_{Y0}−C_{Yi}*C_{X0})]

This vector may be normalized to make it a unit vector.

Normal Line Vector

Normal Line (unit) Vector is the cross product of a line normal to the ABC plane with the Limit Line Vector.

\vec{V}_{Ni}=\vec{V}_{Li}×\vec{C}ᵢ=(V_{NXi},V_{NYi},V_{NZi})

(V_{NXi},V_{NYi},V_{NZi})=[(V_{LYi}*C_{Z0}−V_{LZi}*C_{Y0}),(V_{LZi}*C_{X0}−V_{LXi}*C_{Z0}),(V_{LXi}*C_{Y0}−V_{LYi}*C_{X0})]

This vector may be normalized to make it a unit vector.

Limit Line Constants

Solve the Limit Line Constants by choosing the best variable axis. Look for maximum vector component value of the following equation.

\vec{V}_{Li}·\vec{V}_{Ni}

If(V_{LXi}·V_{NXi}=Max vector component, “X” is variable axis)

If(V_{LYi}·V_{NYi}=Max vector component, “Y” is variable axis)

If(V_{LZi}·V_{NZi}=Max vector component, “Z” as variable axis)

TABLE 9

| Variable Axis | X | Y | Z |
|---|---|---|---|
| ΔK_{Xi} | | V_{LXi}/V_{LYi} | V_{LXi}/V_{LZi} |
| K_{Xi} | | X_{Di} − (V_{LXi}/ | X_{Di} − (V_{LXi}/ |
| | | V_{LYi})*Y_{Di} | V_{LZi})*Z_{Di} |
| ΔK_{Yi} | V_{LYi}/V_{LXi} | | V_{LYi}/V_{LZi} |
| K_{Yi} | Y_{Di} − (V_{LYi}/ | | Y_{Di} − (V_{LYi}/ |
| | V_{LXi})*X_{Di} | | V_{LZi})*Z_{Di} |
| ΔK_{Zi} | V_{LZi}/V_{LXi} | V_{LZi}/V_{LYi} | |
| K_{Zi} | Z_{Di} − (V_{LZi}/ | Z_{Di} − (V_{LYi}/ | |
| | V_{LXi})*X_{Di} | V_{LXi})*Y_{Di} | |

Normal Line Constants

Use the same variable axis as with the Limit Line Constants.

TABLE 10

| Variable Axis | X | Y | Z |
|---|---|---|---|
| ΔN_{Xi} | | V_{NXi}/V_{NYi} | V_{NXi}/V_{NZi} |
| N_{Xi} | | X_{T} − (V_{NXi}/ | X_{T} − (V_{NXi}/ |
| | | V_{NYi})*Y_{T} | V_{NZi})*Z_{T} |
| ΔN_{Yi} | V_{NYi}/V_{NXi} | | V_{NYi}/V_{NZi} |
| N_{Yi} | Y_{T} − (V_{NYi}/ | | Y_{T} − (V_{NYi}/ |
| | V_{NXi})*X_{T} | | V_{NZi})*Z_{T} |
| ΔN_{Zi} | V_{NZi}/V_{NXi} | V_{NZi}/V_{NYi} | |
| N_{Zi} | Z_{T} − (V_{NZi}/ | Z_{T} − (V_{NZi}/ | |
| | V_{NXi})*X_{T} | V_{NYi})*Y_{T} | |

Normal Line Intersect Point

Find Normal Line Intersection Point (X_{Pi}, Y_{Pi}, Z_{Pi}) for each lumen Line crossing the pressure limit. Use the same variable axis as with the Limit Line Constants.

TABLE 11

| Variable Axis | X | Y | Z |
|---|---|---|---|
| X_{Pi} | (N_{Zi} − K_{Zi})/ | K_{Xi} + ΔK_{Xi}*Y_{Pi} | K_{Xi} + ΔK_{Xi}*Z_{Pi} |
| | (ΔK_{Zi} − ΔN_{Zi}) | | |
| Y_{Pi} | K_{Yi} + ΔK_{Yi}*X_{Pi} | (N_{Zi} − K_{Zi})/ | K_{Yi} + ΔK_{Yi}*Z_{Pi} |
| | | (ΔK_{Zi} − ΔN_{Zi}) | |
| Z_{Pi} | K_{Zi} + ΔK_{Zi}*X_{Pi} | K_{Zi} + ΔK_{Zi}*Y_{Pi} | (N_{Yi} − K_{Yi})/ |
| | | | (ΔK_{Yi} − ΔN_{Yi}) |

Limit Lines Intersect Points

Find the pressure Limit Lines (i, k) Intersection points for lumens that cross pressure limit. Use the same variable axis as with the Limit Line Constants.

TABLE 12

| Variable Axis | X | Y | Z |
|---|---|---|---|
| X_{Pik} | (K_{ZXi} − K_{ZXk})/ | K_{XYk} + | K_{XZk} + |
| | (ΔK_{ZXk} − ΔK_{ZXi}) | ΔK_{XYk}*Y_{Pik} | ΔK_{XZk}*Z_{Pik} |
| Y_{Pik} | K_{YXk} + | (K_{ZYi} − K_{ZYk})/ | K_{YZi} + |
| | ΔK_{YXk}* X_{Pik} | (ΔK_{ZYk} − ΔK_{ZYi}) | ΔK_{YZi}*Z_{Pik} |
| Z_{Pik} | K_{ZXk} + | K_{ZYk} + | (K_{YZi} − K_{YZk})/ |
| | ΔK_{ZXk}* X_{Pik} | ΔK_{ZYk}*Y_{Pik} | (ΔK_{YZk} − ΔK_{YZi}) |

Intersect Point Pressure Vectors

Solve the pressure array(s) for each Intersection Point

\vec{Pr}_{Ti}=\vec{C}ᵢ*(X_{Pi},Y_{Pi},Z_{Pi})

\vec{Pr}_{Tik}=\vec{C}ᵢ*(X_{Pik},Y_{Pik},Z_{Pik})

Solve for the intersect points of all limit lines. The number of limit lines will be dictated by the number of lumens that cross a limit pressure. The max number possible for two segments is six lumens lines.

In Table 13 below the i and k indicate a specific lumen combination where i and k are not be the same number and all combinations should be selected only once.

TABLE 13
i

| | | 1 | 2 | 3 | 4 | 5 | 6 |
|---|---|---|---|---|---|---|---|
| | 1 | | x | x | x | x | x |
| | 2 | | | x | x | x | x |
| k | 3 | | | | x | x | x |
| | 4 | | | | | x | x |
| | 5 | | | | | | x |
| | 6 | | | | | | |

This chart indicates the number of lumen line intersect points as a function of the number of lumen lines in play. Note that with six lumen lines there are 15 intersect points as indicated by the “x's”. Add this to the Normal line intersects, with six lumens over the pressure limit a total of 21 intersect points (6 normal+15 lumen line) would benefit from being resolved.

Now find the closest (and achievable) point Q_{d} to the target Q_{T} (with all pressure values within the limits). There should be one or more intersect points that are within the pressure limits. To optimize the search sequence, note the following.
An achievable normal intersection points will be closer than any achievable lumen line intersection points. The Normal intersection point comes the normal line through the Target point.
The farthest Normal intersection point will generally be the closest Normal point that can be achieved (within the pressure limits). If it is not within the pressure limits, the closest point will be one of the lumen line intersection points.
It may be possible for a lumen which is not pressure limited across Q_{A}, Q_{B}, or Q_{C} to be an intersect limit line that defines the closest point.

Line Mode

Pressure Limit Points

Referring now to FIG. 26G, for lumens that are not within a pressure limit, find the points on lines A-B, B-C, and C-A that cross the limit pressure line. These points will all be in a line in space.

First, determine if pressure limit plane is normal to the A-B-C plane, in which case the pressure parallel to the allowable direction of motion and there may be no solution. For this condition return the previous telemetry.

ΔPr_{ABC}=Pr_{AB}−Pr_{BC}

ΔPr_{BCA}=Pr_{BC}−Pr_{CA}

ΔPr_{CAB}=Pr_{CA}−Pr_{AB}

MinΔPr=0.001 (Other numbers may be determined empirically or analytically.)

Second, determine which limit points are farthest apart. This avoids using two points with the same pressure which leads to a non-zero divisor when finding the points.
Flag=IF(MAX(ΔPr_{ABC},ΔPr_{BCA},ΔPr_{CAB})<MinΔPr,“Normal”,
   IF(AND(ABS(ΔPr_{ABC})>=ABS(ΔPr_{BCA}),(ΔPr_{ABC})>=ABS(ΔPr_{CAB})),“AB”,
   IF(ΔPr_{BCA}>=ABS(ΔPr_{CAB}),“BC”,
   “CA”)))=
=IF(Fla “Normal”,<return previous telemetry>,
   IF(Flag=“AB”,

(X_{Di},Y_{Di},Z_{Di})=(Pr_{Limit}−Pr_{Ai})/(Pr_{Bi}−Pr_{Ai})*[(X_{B},Y_{B},Z_{B})−(X_{A},Y_{A},Z_{A})]+(X_{A},Y_{A},Z_{A}),
IF(Flag=“BC”,

(X_{Di},Y_{Di},Z_{Di})=(Pr_{Limit}−Pr_{Bi})/(Pr_{Ci}−Pr_{Bi})*[(X_{C},Y_{C},Z_{C})−(X_{B},Y_{B},Z_{B})]+(X_{B},Y_{B},Z_{B}),
IF(Flag=“CE”,

(X_{Di},Y_{Di},Z_{Di})=(Pr_{Limit}−Pr_{Ci})/(Pr_{Ai}−Pr_{Ci})*[(X_{A},Y_{A},Z_{A})−(X_{C},Y_{C},Z_{C})]+(X_{C},Y_{C},Z_{C}),
<missing flag>))))

Pressure Limit Line

Find the pressure Limit Line (unit) Vector with the cross product of the pressure and A-B-C plane's normal vectors, which can be taken directly from the plane constants above.

\vec{V}_{L}=(V_{LXi},V_{LYi},Z_{LZi})=(C_{Xi},C_{Yi},C_{Zi})×(C_{X0},C_{Y0},C_{Z0})

(V_{LXi},V_{LYi},V_{LZi})=[(C_{Yi}*C_{Z0}−C_{Zi}*C_{Y0}),(C_{Zi}*C_{X0}−C_{Xi}*C_{Z0}),(C_{Xi}*C_{Y0}−C_{Yi}*C_{X0})]

This vector may be normalized to make it a unit vector.

Normal Line Vector

Normal Line (unit) Vector is the cross product of a line normal to the ABC plane with the Limit Line Vector.

\vec{V}_{N}=(V_{NX},V_{NY},V_{NZ})=[(X_{A}−X_{T}),(Y_{A}−X_{T}),(Z_{A}−Z_{T})]

This vector may be normalized to make it a unit vector.

Limit Line Constants

Solve the Limit Line Constants by choosing the best variable axis. Look for maximum vector component value of the following equation.

\vec{V}_{Li}·\vec{V}_{N}

If(V_{LXi}·V_{NX}=Max vector component, “X” is variable axis)

If(V_{LYi}·V_{NY}=Max vector component, “Y” is variable axis)

If(V_{LZi}·V_{NZ}=Max vector component, “Z” as variable axis)

Limit line Constants:

TABLE 14

| Variable Axis | X | Y | Z |
|---|---|---|---|
| ΔK_{Xi} | | V_{LXi}/V_{LYi} | V_{LXi}/V_{LZi} |
| K_{Xi} | | X_{Di} − (V_{LXi}/ | X_{Di} − (V_{LXi}/ |
| | | V_{LYi})*Y_{Di} | V_{LZi})*Z_{Di} |
| ΔK_{Yi} | V_{LYi}/V_{LXi} | | V_{LYi}/V_{LZi} |
| K_{Yi} | Y_{Di} − (V_{LYi}/ | | Y_{Di} − (V_{LYi}/ |
| | V_{LXi})*X_{Di} | | V_{LZi})*Z_{Di} |
| ΔK_{Zi} | V_{LZi}/V_{LXi} | V_{LZi}/V_{LYi} | |
| K_{Zi} | Z_{Di} − (V_{LZi}/ | Z_{Di} − (V_{LYi}/ | |
| | V_{LXi})*X_{Di} | V_{LXi})*Y_{Di} | |

Normal Line Constants

Solve Normal Line Constants (line normal to Smart Plane). Use the same variable axis as with the Limit Line Constants.

TABLE 15

| Variable Axis | X | Y | Z |
|---|---|---|---|
| ΔN_{X} | | V_{NX}/V_{NY} | V_{NX}/V_{NZ} |
| N_{X} | | X_{T} − (V_{NX}/ | X_{T} − (V_{NX}/ |
| | | V_{NY})*Y_{T} | V_{NZ})*Z_{T} |
| ΔN_{Y} | V_{NY}/V_{NX} | | V_{NY}/V_{NZ} |
| N_{Y} | Y_{T} − (V_{NY}/ | | Y_{T} − (V_{NY}/ |
| | V_{NX})*X_{T} | | V_{NZ})*Z_{T} |
| ΔN_{Z} | V_{NZ}/V_{NX} | V_{NZ}/V_{NY} | |
| N_{Z} | Z_{T} − (V_{NZ}/ | Z_{T} − (V_{NZ}/ | |
| | V_{NX})*X_{T} | V_{NY})*Y_{T} | |

Find Normal Line and pressure Limit Lines intersection points (X_{Pi}, Y_{Pi}, Z_{Pi}) for each lumen Line crossing the pressure limit. Use the same variable axis as with the Limit Line Constants.

TABLE 16

| Variable Axis | X | Y | Z |
|---|---|---|---|
| X_{Pi} | (N_{Zi} − K_{Zi})/ | K_{Xi} + ΔK_{Xi}*Y_{Pi} | K_{Xi} + ΔK_{Xi}*Z_{Pi} |
| | (ΔK_{Zi} − ΔN_{Zi}) | | |
| Y_{Pi} | K_{Yi} + ΔK_{Yi}*X_{Pi} | (N_{Zi} − K_{Zi})/ | K_{Yi} + ΔK_{Yi}*Z_{Pi} |
| | | (ΔK_{Zi} − ΔN_{Zi}) | |
| Z_{Pi} | K_{Zi} + ΔK_{Zi}*X_{Pi} | K_{Zi} + ΔK_{Zi}*Y_{Pi} | (N_{Yi} − K_{Yi})/ |
| | | | (ΔK_{Yi} − ΔN_{Yi}) |

Solve the pressure array(s) for each Intersection Point

\vec{Pr}_{Ti}=\vec{C}ᵢ*(X_{Pi},Y_{Pi},Z_{Pi})

Solve for the intersect points of all limit lines. The number of limit lines will be dictated by the number of lumens that cross a limit pressure. The max number for two segments may be six lumens lines.
Now find the closest (achievable) point Q_{d} to the target Q_{T} (with all pressure values within the limits). There should be one or more intersect points that are within the pressure limits.

Setting Up the Pressure Gradient with Fixed Position

General Model and Steps Towards Finding the Closest Gradient Solution

Referring once again to FIG. 26D, points T, A, B, and C are located at the same position (graphically separated for conceptualizing) but with different spin (α) and pitch (β) angles than the goal vector Q_{T}. The commanded rotation is defined in the direction from Q_{A} to Q_{T}. Each tip point has unique orientation associated with the Q vectors. The Q position values (X, Y, Z) for each Q vector are the same which allows for two unique orientation (βₓ, β_{y}) at three different pressure values (per lumen). The simulation module derives Q_{A}, Q_{B}, and Q_{C} tip vectors based the desired Q_{T} input, and sends the three Q vectors to the pressure control module. The pressure control module solves for the lumen pressure (Pr_{A}, Pr_{B}, Pr_{C}) for each Q position. A lumen pressure gradient between the Q positions is generated. The lumen pressure vector at the target point (\vec{Pr}_{T}) is solved using this gradient. If \vec{Pr}_{T} has one or more lumen pressures outside the lumen pressure limits, the gradient equations are used to find the closest alternative orientation (βₓ, β_{y}) that is within pressure limit.

Symbols
Q_{O}—current tip position in world space.
Q_{T}—goal orientation (Tip Input) in world space.
Q_{A}, Q_{B}, Q_{C}—same Q_{T} (X,Y,Z) position; equal angular displacement from Q_{T}.
\vec{Pr}—vector containing each lumen pressure at a specific Q position.
d_{T}—is the orientational target offset.

Gradient Model

The following Gradient Math occurs in the pressure control module after receiving the Q vectors from the simulation module. This gradient model applies to the Gimbal and Axial Mode.

Spatial Plane

Find the plane formed by the positions of Q_{A}, Q_{B}, and Q_{C}, using the βₓ, and β_{y}, component.

C_{X0}*(βX−βx_{A})+C_{Y0}*(βy−βy_{A})=0

C_{X0}*βx+C_{Y0}*βy=C_{X0}*βx_{A}+C_{Y0}*βy_{A} (1)
C_{X0}, and C_{Y0}, are the plane constants (C_{Z0}=0) associated with points A, B, and C. A vector formed by (C_{X0}, C_{Y0}, C_{Z0}) is perpendicular to plane.
X_{A}, Y_{A}, Z_{A} are known position points of Q_{A} and can be any known point.
X, Y, & Z are the Q position coordinate variables.

Pressure Plane

Linear gradient for the lumen pressure variance throughout the Q sample range. Use the following formulas to solve for the planer “C” constants.

C_{Xi}*βx+C_{Yi}*βy=Prᵢ [2]
“i” represents the lumen number; for a two-segment system “i” is from 1 through 6.
C_{Xi} and C_{Yi} are the pressure constants to estimate the pressure of lumen “i”.
βx and βy are the Q orientation coordinate variables.
Prᵢ is the estimated pressure of lumen “i” at orientation βx and βy.

For each of the three Q's defined by A, B, and C, two orientations (βxᵢ, βyᵢ) and Lumen Pressure (Prᵢ) are known. Set up equation and solve for constants for each lumen pressure.

C_{Xi}*βx_{A}+C_{Yi}*βy_{A}=Pr_{Ai}

C_{Xi}*βx_{B}+C_{Yi}*βy_{B}=Pr_{Bi}

C_{Xi}*βx_{C}+C_{Yi}*βy_{C}=Pr_{Ci}

$$[ β ] \cdot {\vec{C}}_{i} = {\vec{P r}}_{i} \\ [ β ] = ( \begin{matrix}{β x}_{A} & {β y}_{A} \\ {β x}_{B} & {β y}_{B} \\ {β x}_{C} & {β y}_{C}\end{matrix} ) \\ {\vec{C}}_{i} = {[ β ]}^{- 1} \cdot {\vec{P r}}_{i}$$

Since there are more Q points than variables, a least square fit may be applied for a pseudo inverse matrix.

\vec{C}ᵢ=([β]·[β]^{T})⁻¹·[β]^{T}·\vec{Pr}ᵢ

Goal Position

Geometrically, Q_{T} is the average of Q_{A}, Q_{B}, and Q_{C} and expressed as follows:

\vec{Q}_{T}=(\vec{Q}_{A}+\vec{Q}_{B}+\vec{Q}_{C})/3

βx_{T}=(βx_{A}+βx_{B}+βx)/3; βy_{T}=(βy_{A}+βy_{B}+βy_{C})/3

Goal Lumen Pressure

The pressure vector can be found through the “C” constants vector:

\vec{Pr}ᵢ=\vec{C}=ᵢ·\vec{Q}_{T}

Use equation 2 (6 times for two segments) and solve for the target lumen pressures \vec{Pr}_{T} (at position Q_{T}).
If all lumen pressures at \vec{Pr}_{T} are within the pressure limits, use the current pressure vector. If one or more pressure components are outside the limits, solve for the closest position on the Smart Plane where all pressures are within the pressure limits.

Pressure Limit Points

For lumens that are not within a pressure limit, find the points on graphic lines A-B, B-C, and C-A that cross the limit pressure line. These points will all be one a graphic line.

First, determine if pressure limit plane is normal to the A-B-C plane, in which case the pressure parallel to the allowable direction of motion and there may be no solution. For this condition return the previous telemetry.

ΔPr_{ABC}=Pr_{AB}−Pr_{BC}

ΔPr_{BCA}=Pr_{BC}−Pr_{CA}

ΔPr_{CAB}=Pr_{CA}−Pr_{AB}

MinΔPr=0.001 (Other numbers may be determined empirically or analytically.)

Second, determine which limit points are farthest apart. This avoids using two points with the same pressure which leads to a non-zero divisor when finding the points.
Flag=IF(MAX(ΔPr_{ABC},ΔPr_{BCA},ΔPr_{CAB})<MinΔPr,“Normal”,
   IF(AND(ABS(ΔPr_{ABC})>=ABS(ΔPr_{BCA}),(ΔPr_{ABC})>=ABS(ΔPr_{CAB})),“AB”,
   IF(ΔPr_{BCA}>=ABS(ΔPr_{CAB}),“BC”,
   “CA”)))=
=IF(Flag=“Normal”,<return previous telemetry>,
   IF(Flag=“AB”,

(βx_{Di},βy_{Di})=(Pr_{Limit}−Pr_{Ai})/(Pr_{Bi}−Pr_{Ai})*[(βx_{B},βy_{B})−(βx_{A},βy_{A})]+(βy_{A},βy_{A}),
IF(Flag=“BC”,

(βx_{Di},βy_{Di})=(Pr_{Limit}−Pr_{Bi})/(Pr_{Ci}−Pr_{Bi})*[(βx_{C},βy_{C})−(βx_{B},βy_{B})]+(βx_{B},βy_{B}),
IF(Flag=“CE”,

(βx_{Di},βy_{Di})=(Pr_{Limit}−Pr_{Ci})/(Pr_{Ai}−Pr_{Ci})*[(βx_{A},βy_{A})−(βx_{C},βy_{C})]+(βx_{C},βy_{C}),
<missing flag>))))

Pressure Limit Line

Find the pressure Limit Line Vector. The Limit Line Vector is perpendicular to the Pressure constants Vector (C_{Xi}, C_{Yi}). Since they are perpendicular, the dot product of the “C” Vector and Limit Line Vector is equal to zero.

\vec{V}_{Li}·\vec{C}ᵢ=[V_{LXi},V_{LYi}]·(C_{Xi},C_{Yi})=0

\vec{V}_{Li}=[V_{LXi},V_{LYi}]=(C_{Yi},−C_{Xi})

This vector may be normalized to make it a unit vector.

Gimbal Mode

Gimbal Mode control allows change in two axes of orientations at a fixed position.

When orientation adjustments meet a boundary, the rotation slides along the angle boundary. The method maintains telemetry on a point while moving to the closest orientation.

Normal Line Vector

In Gimbal Mode, the Normal Line Vector is a line that passes through the goal point Q_{T} and is perpendicular to the Limit Line Vector. Since they are perpendicular, the dot product of the Normal Line Vector and Limit Line Vector is equal to zero.

\vec{V}_{Li}·\vec{V}_{Ni}=[C_{Yi},−C_{Xi}]·(V_{NXi},V_{NYi})=0

\vec{V}_{Ni}=(V_{NXi},V_{NYi})=(C_{Xi},C_{Yi})

This vector may be normalized to make it a unit vector.

Limit Line Constants

Solve the Limit Line Constants by choosing the best variable axis. Look for maximum vector component value of the following equation.

\vec{V}_{Li}·\vec{V}_{Ni}

If(V_{LXi}·V_{NXi}=Max vector component, “βx” is variable axis)

If(V_{LYi}·V_{NYi}=Max vector component, “βx” is variable axis)

Limit line Constants:

TABLE 17

| Variable Axis | βx | βy |
|---|---|---|
| ΔK_{Xi} | | V_{LXi}/V_{LYi} |
| K_{Xi} | | X_{Di} − (V_{LXi}/V_{LYi})*Y_{Di} |
| ΔK_{Yi} | V_{LYi}/V_{LXi} | |
| K_{Yi} | Y_{Di} − (V_{LYi}/V_{LXi})*X_{Di} | |

Normal Line Constants

Solve Normal Graphic Line Constants. Use the same variable axis as with the Limit Line Constants.

TABLE 18

| Variable Axis | βx | βy |
|---|---|---|
| ΔN_{Xi} | | V_{NXi}/V_{NYi} |
| N_{Xi} | | X_{T} − (V_{NXi}/V_{NYi})*Y_{T} |
| ΔN_{Yi} | V_{NYi}/V_{NXi} | |
| N_{Yi} | Y_{T} − (V_{NYi}/V_{NXi})*X_{T} | |

Normal Line Intersect Point

Find Normal Line and pressure Limit Lines intersection points ((βx_{Pi}, βy_{Pi}) for each lumen Line crossing the pressure limit. Use the same variable axis as with the Limit Line Constants.

| Variable Axis | βx | βy |
|---|---|---|
| βx_{Pi} | (N_{Yi} − K_{Yi}/(ΔK_{Yi} − | K_{Xi} + ΔK_{Xi}*Y_{Pi} |
| | ΔN_{Yi}) | |
| βy_{Pi} | K_{Yi} + ΔK_{Yi}*X_{Pi} | (N_{Xi} − K_{Xi})/(ΔK_{Xi} − |
| | | ΔN_{Xi}) |

Limit Lines Intersect Points

Find the pressure Limit Lines (i, k) Intersection points for lumens that cross pressure limit. Use the same variable axis as with the Limit Line Constants.

| Variable Axis | βx | βy |
|---|---|---|
| βx_{Pik} | (K_{ZXi} − K_{ZXk})/(ΔK_{ZXk} − | K_{XYk} + ΔK_{XYk}*Y_{Pik} |
| | ΔK_{ZXi}) | |
| βy_{Pik} | K_{YXk} + ΔK_{YXk}* X_{Pik} | (K_{ZYi} − K_{ZYk})/(ΔK_{ZYk} − |
| | | ΔK_{ZYi}) |

Intersect Point Pressure Vectors

Solve the pressure array(s) for each Intersection Point

\vec{Pr}_{Ti}=\vec{C}ᵢ*(βx_{Pi},βy_{Pi})

\vec{Pr}_{Tik}=\vec{C}ᵢ*(βx_{Pik},βy_{Pik})

Solve for the intersect points of all limit lines. The number of limit lines will be dictated by the number of lumens that cross a limit pressure. The max number possible for two segments is six lumens lines.
In the chart below the i and k indicate a specific lumen combination where i and k are not be the same number and all combinations should only be selected once.

TABLE 19
i

| | | 1 | 2 | 3 | 4 | 5 | 6 |
|---|---|---|---|---|---|---|---|
| | 1 | | x | x | x | x | x |
| | 2 | | | x | x | x | x |
| k | 3 | | | | x | x | x |
| | 4 | | | | | x | x |
| | 5 | | | | | | x |
| | 6 | | | | | | |

Table 19 indicates the number of lumen line intersect points as a function of the number of lumen lines in play. Note that with six lumen lines there are 15 intersect points as indicated by the “x's”. Add this to the Normal line intersects, with six lumens over the pressure limit a total of 21 intersect points (6 normal+15 lumen line) would benefit from being resolved.

Now find the closest (and achievable) point Q_{d} to the target Q_{T} (with all pressure values within the limits). There should be one or more intersect points that are within the pressure limits. To optimize the search sequence, note the following.
An achievable normal intersection points will be closer than any achievable lumen line intersection points. The Normal intersection point comes the normal line through the Target point.
The farthest Normal intersection point will always be the closest Normal point that can be achieved (within the pressure limits). If it is not within the pressure limits, the closest point will be one of the lumen line intersection points.
It may be possible, though unlikely, for a lumen which is not pressure limited across Q_{A}, Q_{B}, or Q_{C} to be an intersect limit line that defines the closest point. The current math does not consider this condition and assumes this will not occur.

Axial Mode

Axial Mode control allows change in orientation at a fixed position and about one axis.

When orientation adjustments meet a boundary, pitch is sacrificed in order to meet circumferential angle about the Normal axis. The method maintains telemetry on a point while moving to the closest orientation while sacrificing pitch angle.

Normal Line Vector

For the Axial Mode the Normal Line Vector is the line normal to the ABC plane and, assuming point A is on trajectory path, it can be found by the orientation vector between point A and T.

\vec{V}_{N}=(V_{NX},V_{NY})=[(βx_{A}·βx_{T}),(βy_{A}−βy_{T})]

This vector may be normalized to make it a unit vector.

Limit Line Constants

Solve the Limit Line Constants by choosing the best variable axis. Look for maximum vector component value of the following equation.

\vec{V}_{Li}·\vec{V}_{N}

If(V_{LXi}·V_{NX}=Max vector component, “βx” is variable axis)

If(V_{LYi}·V_{NY}=Max vector component, “βy” is variable axis)

Limit line Constants:

TABLE 20

| Variable Axis | βx | βy |
|---|---|---|
| ΔK_{Xi} | | V_{LXi}/V_{LYi} |
| K_{Xi} | | X_{Di} − (V_{LXi}/V_{LYi})*Y_{Di} |
| ΔK_{Yi} | V_{LYi}/V_{LXi} | |
| K_{Yi} | Y_{Di} − (V_{LYi}/V_{LXi})*X_{Di} | |

Normal Line Constants

Solve Normal Graphic Line Constants. Use the same variable axis as with the Limit Line Constants.

TABLE 21

| Variable Axis | βx | βy |
|---|---|---|
| ΔN_{X} | | V_{NX}/V_{NY} |
| N_{X} | | X_{T} − (V_{NX}/V_{NY})*Y_{T} |
| ΔN_{Y} | V_{NY}/V_{NX} | |
| N_{Y} | Y_{T} − (V_{NY}/V_{NX})*X_{T} | |

Find Normal Line and pressure Limit Lines intersection points (X_{Pi}, Y_{Pi}, Z_{Pi}) for each lumen Line crossing the pressure limit. Use the same variable axis as with the Limit Line Constants.

| Variable Axis | βx | βy |
|---|---|---|
| βx_{Pi} | (N_{Yi} − K_{Yi})/(ΔK_{Yi} − | K_{Xi} + ΔK_{Xi}*Y_{Pi} |
| | ΔN_{Yi}) | |
| βy_{Pi} | K_{Yi} + ΔK_{Yi}*X_{Pi} | (N_{Xi} − K_{Xi})/(ΔK_{Xi} − |
| | | ΔN_{Xi}) |

Solve the pressure array(s) for each Intersection Point

\vec{Pr}_{Ti}=Cᵢ*(βx_{Pi},βy_{Pi})

Now find the closest (achievable) point Q_{d} to the target Q_{T} (with all pressure values within the limits). There should be one or more intersect points that are within the pressure limits.

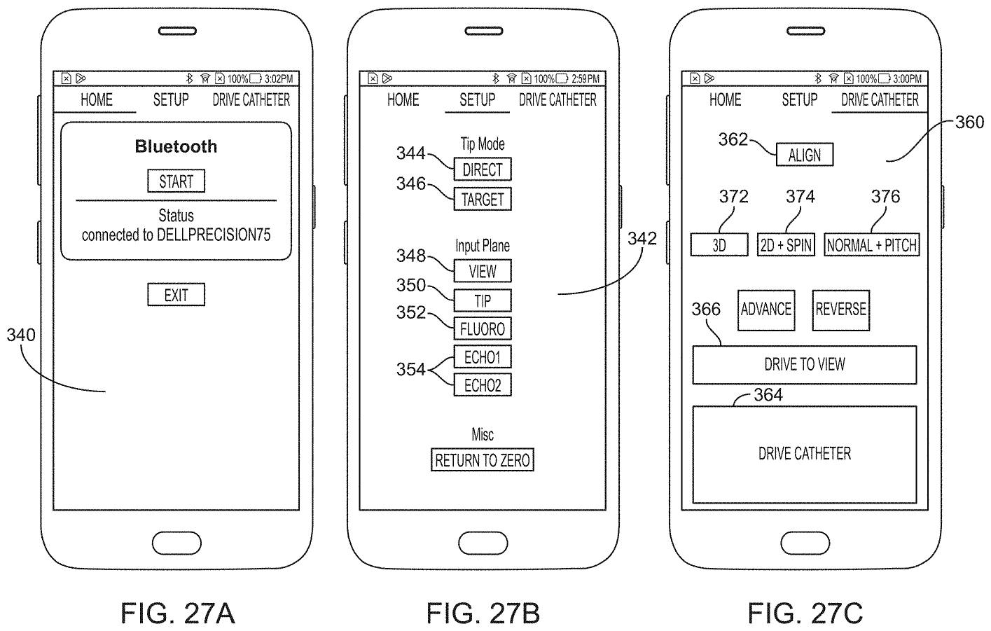

Referring now to FIGS. 1 and 27A-27C user interface pages of a mobile computing device configured for use as 6 DOF input 16 are shown. The exemplary mobile computing device comprises a Tango™-compatible ASUS ZenfoneAR™ running an Android™ operating system, although alternative augmented reality (AR) capable mobile computing devices configured for ARCore™, ARKit™, and/or other AR packages. A Home page 340 shown in FIG. 27A includes buttons associated with establishing or terminating Bluetooth™, WiFi, or other wireless communications with other components of the processor, with the buttons and underlying functionality being configured with security and identification protocols that inhibit malicious or inadvertent interference with the use of the articulation system. A Setup page 342 includes alternatively selectable Direct mode button 344 and Target mode button 346 that can be used to alternate the processor mode between a Drive mode that is configured for real-time driving of the catheter in response to movement commands, and a Target mode that is configured for driving of a virtual or phantom catheter, as described above. Setup page 342 also includes a number of alternatively selectable buttons associated with planes to which articulation modes may be constrained. The input plane buttons include a View plane button 348 which references the plane of display 206 (see FIG. 19). A Tip plane button 350 references a plane normal to the tip of the catheter. A fluoro plane button 352 references a 2D image capture plane of the fluoro system, while Echo plane buttons 354 each reference a 2D image plane of the fluoro system.

Referring now to FIG. 27C, Drive Catheter page 360 includes an Align button 362 which is configured to align the input space of the input device 16 with the display reference frame 208. For example, the user can orient the top end of the mobile computing device toward (parallel to) the display plane with the screen of the mobile device oriented upward and the elongate axis of the mobile device perpendicular to the display plane, and then engage the Align button. The orientation of the mobile device during engagement of the Align button can be stored, and subsequent input commands entered by, for example, engaging a Drive Catheter button 364 and moving the mobile device from a starting location and orientation (with the Drive Catheter button engaged) can be transformed to the display frame using standard quaternion operations (regardless of the specific starting orientation and location). Processor 214 can use this input to induce movement of the catheter (or a phantom catheter) as seen in an image shown in the display, to translate and rotate in correlation with the movement of the mobile device. Release of the Drive Catheter button can then decouple the input device 16 from the catheter. A Drive View button 366, when engaged, provides analogous coupling of the mobile device to a 2D, 3D, or hybrid 2D/3D image presented on the display so that the image (including the visible portions of the catheter and the displayed tissue) translate and rotate in correlation with movement of the mobile device as if the mobile device was coupled to the catheter tip, thereby allowing the user to view the image from different locations and/or orientations. Advance and retract buttons 368, 370 induce movement of the catheter along a trajectory between a first pose and a second or phantom pose, as described above. Alternatively selectable mode buttons, including a 3D mode button 372, a Planar-and-spin mode button 374, and a Normal-and-pitch mode button 376 can be used to select between unconstrained motion and motion constrained relative to the plane selected on the Setup page 342 as described above with reference to FIG. 27B.

Referring now to FIGS. 16A-16D, 19, and 25, the system 202 will often have an overall processor 214 that includes a first module such as an input command module 216 configured to receive input from the user for moving a virtual image 146 of the elongate body from a first pose 140 to a second pose 142 on the display 130. The processor will often also have a second module (such as a second input module 216) configured to receive a movement command, and in response, to drive actuators (see, e.g., balloons 42 in FIGS. 2 and 3A-3C) so as to move the elongate body along a trajectory 150 between the first pose and the second pose.

As seen in FIG. 19, system 202 will typically include one or more image capture system coupled to the display, such as fluoro system 236 and/or echo system 238 (typically with an ICE probe, a TEE probe, a trans-thoracic echocardiograpy (TTE) probe, and/or the like). The input module 230, simulation module 256, and pressure control module 230 can work together to move the virtual image 146 of the receptacle and elongate body relative to a stored image of the internal surgical site shown on display 150. One or more of these components of the processor can be configured to transmit image capture commands to the image capture system in response to the same command used to induce movement of the actual elongate body along the trajectory, so that the image capture system selectively images the elongate body only shortly before initiating movement along the trajectory, during some or all of the time the elongate body is between the starting and stopping poses, and/or just after the elongate body has reached the desired pose. By establishing the target pose with reference to one or more still image and then acquiring imaging (particularly fluoro imaging) associated with the move irradiation of the patient, system user, and any other nearby medical professionals can be significantly reduced (as compared to other approaches). Optionally, by superimposing the virtual image on the display of the actual elongate body the user may be presented with a continuously available basis for image guidance and movement verification despite only intermittently imaging the elongate body between the poses. Note that despite such intermittent imaging the processor can still take advantage of image processing module 234 to track the movement of the elongate body using the intermittent images and the virtual image.

Referring now to FIGS. 16A-16D, 19, and 21, and 25, the system may optionally include a first image capture device (such as fluoro system 236) and a second image capture device (such as echo system 238) for generating first image data and second image data, respectively. To register display 206 to both image capture devices processor 214 may include a first registration module (optionally making use of select elements of constraint module 326) and a second registration module (again making use of elements of constraint module 326). Preferably, the first module will be configured for aligning the virtual image 144 of the elongate body with the first image of the elongate body, such as by translating (X-Y-Z) a distal tip of the virtual image on the display into alignment with the image of the actual catheter, spinning the virtual image into alignment, aligning a pitch of the virtual image, and rolling the virtual image, with some or all of the individual axes alignments being independent of the other axes alignments. The second registration module can be configured for aligning the second image of the elongate body with the virtual image, allowing independent modification of the registration of the second image modality without altering the registration of the first imaging modality. Once again, the second module may allow independent modifications to the individual axes of alignment between the virtual image and the second image.

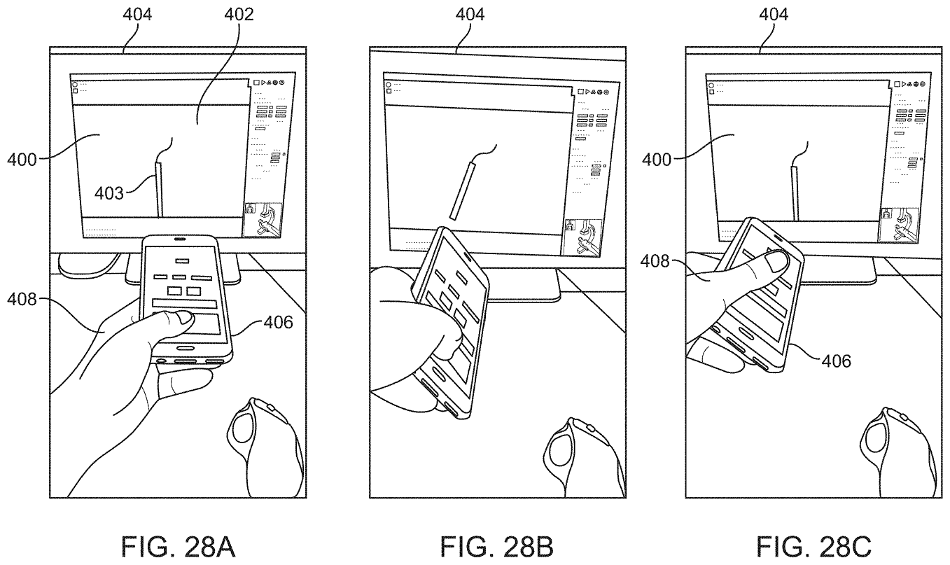

Referring now to FIGS. 28A-28C, manipulation of an image 400 of a 3D workspace 402 and/or a catheter 403 shown on a 2D display 404 using a 6 DOF input device 406 can be understood. Note that the view of the virtual workspace may optionally be driven here without inducing any actual changes in the position or shape of the virtual or actual camera, for example, to allow the user to see the shape of the catheter from a different orientation, or to see a position of the catheter relative to a nearby tissue along a different view axis, or to more clearly see a 2D planar image within a hybrid workspace, or the like. In this example, the system is in a springback mode that allows driving of the view to new positions and orientations, and that returns or springs the view back to the initial position after the drive command has ended. In other modes, the view remains in the position and orientation at the end of the view movement allowing a series of incremental view changes.

Referring now to FIG. 28A, prior to initiation of movement of the view the hand 408 of the user moves the input device 406 into a convenient position and orientation relative to the image of the catheter or other structure shown in the display. The user can initiate a movement command by actuating a drive view input button of the input device, and while the drive view button remains engaged, can move the input device 406 relative to the display 404 (see FIG. 28B). The image 400 shown on display 404 preferably changes in position and orientation in correlation with the movement of the input device 406, giving the user the impression of grasping the virtual and/or hybrid scene in the display and changing the users line of sight without inducing movement of the catheter or other structures seen in the display, and optionally without movement of any image capture device(s) providing any 2D or 3D image data included in image 400. As seen in FIG. 28C, when the hand 408 releases the drive view button of input device 406 the view orientation of the image shown in the display 404 returns back to its position at the start of the movement, with the speed of this spring back preferably being moderate to avoid user disorientation.

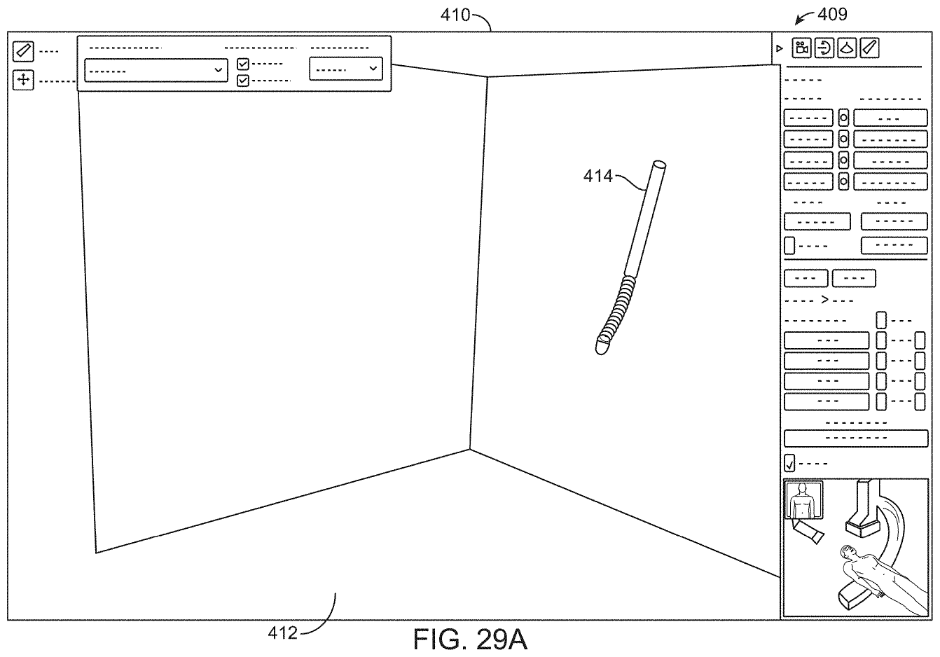

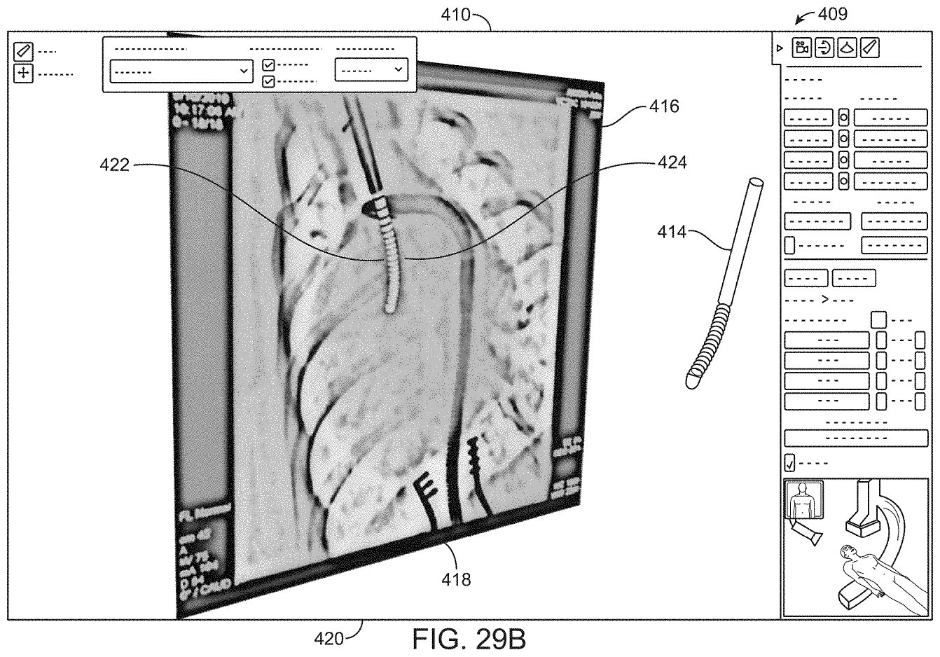

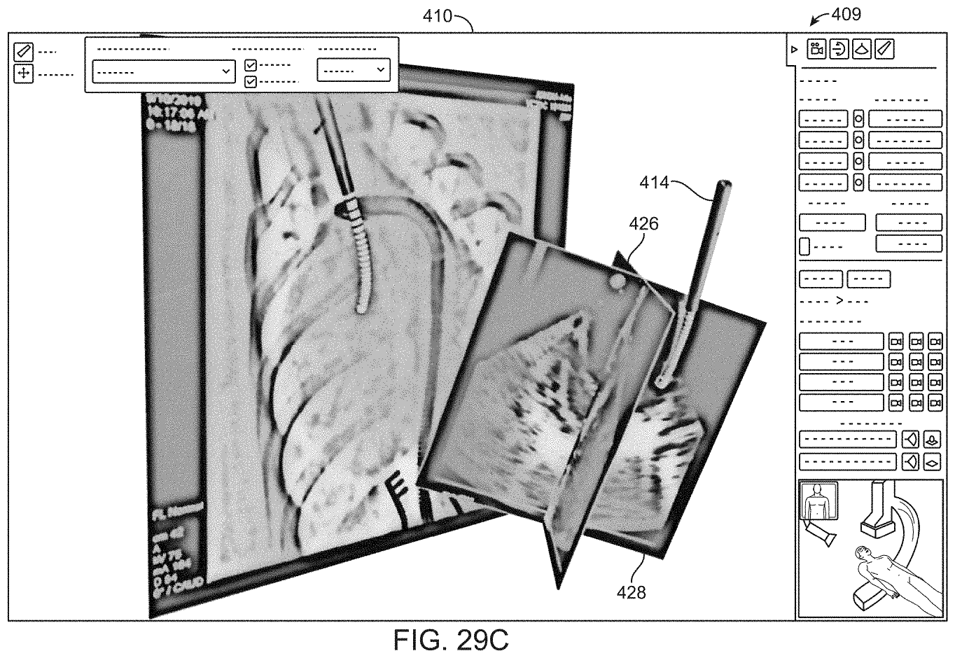

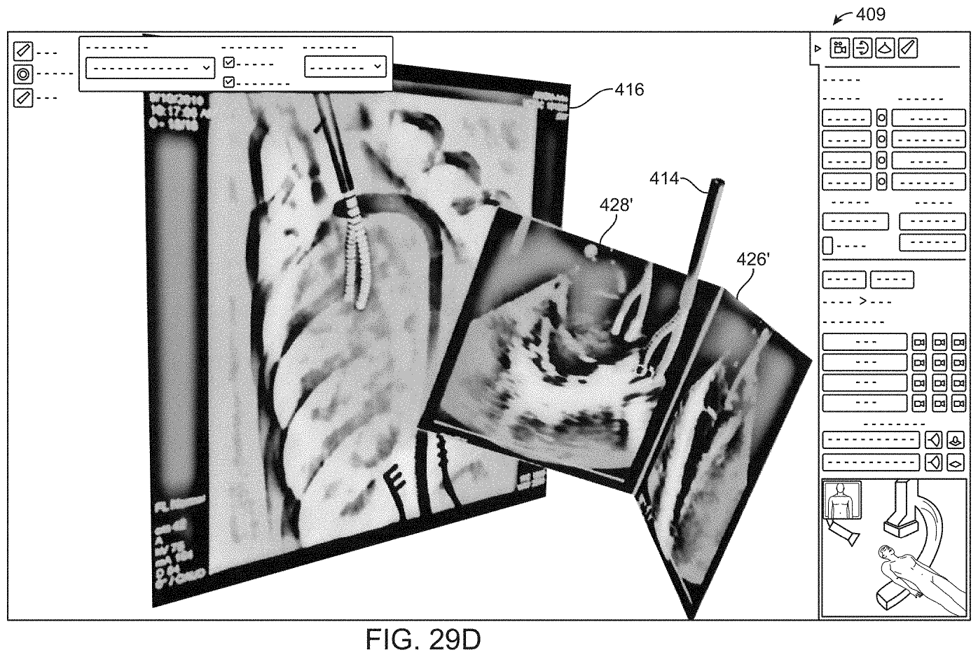

Referring now to FIGS. 29A-29D, components described above may be included in a hybrid 2D/3D image to be presented to a system user on a display 410, with the image components generally being presented in a virtual 3D workspace 412 that corresponds to an actual therapeutic workspace within a patient body. A 3D virtual image of a catheter 414 defines a pose in workspace 412, with the shape of the catheter often being determined in response to pressure and/or other drive signals of the robotic system, in response to imaging, electromagnetic, or other sensor signals so that the catheter image corresponds to an actual shape of an actual catheter. Similarly, a position and orientation of the 3D catheter image 414 in 3D workspace 412 corresponds to an actual catheter based on drive and/or feedback signals.

Referring still to FIGS. 29A-29D, additional elements which may optional be included in image 409 such as a 2D fluoroscopic image 416, the fluoro image having an image plane 418 which may be shown at an offset angle relative to a display plane 420 of image 410 so that the fluoro image and the 3D virtual image of catheter 414 correspond in the 3D workspace. Fluoro image 416 may include an actual image 422 of an actual catheter in the patient, as well as images of adjacent tissues and structures (including surgical tools). A virtual 2D image 424 of 3D virtual catheter 414 may be projected onto the fluoro image 416 as described above. As seen in FIG. 29C, transverse or X-plane planar echo images 426, 428 may similarly be included in hybrid image 409 at the appropriate angles and locations relative to the virtual 3D catheter 414, with 2D virtual images optionally being projected thereon. However, as shown in FIG. 29D, it will often be advantageous to offset the echo image planes from the virtual catheter to generate associated offset echo images 426′, 428′ that can more easily be seen and referenced while driving the actual catheter. The planar fluoro and echo images within the hybrid image 409 will preferably comprise streaming live actual video obtained from the patient when the catheter is being driven.

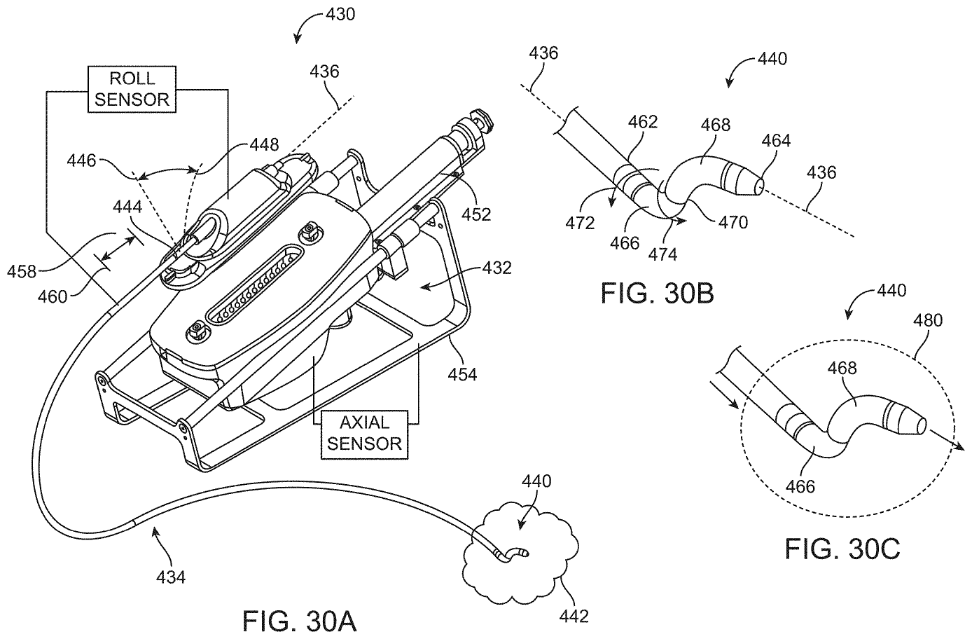

Referring now to FIGS. 30A-30C the proximal catheter housing and/or driver support structures will optionally be configured to both allow and sense manual manipulation of the catheter body outside the patient, and to drive the articulating tip in response to such manipulations so as to inhibit changes in position of the tip. More specifically, a catheter system 430 includes many of the components described above, including a driver 432 detachably receiving a catheter 434 having a flexible catheter body extending along an axis 436. A passive or un-driven proximal catheter body 438 extends distally to an actively driven portion 440 configured for use in an internal surgical site 442 within a patient body. A rotational handle 444 adjacent a proximal housing of the catheter allows the catheter body to be rotated relative to the driver about the catheter axis from a first rotational orientation 446 to a second rotational orientation 448, with the rotation being sensed by a roll sensor 450. An axial adjustment mechanism 452 couples the driver 432 to a driver support 545, and an axial sensor 456 senses changes in axial position of the catheter body when the mechanism is actuated manually by the user, for example to move between a first axial location 458 and a second axial location 460. Resulting rotation and/or axial translation of the catheter body induces corresponding rotation and/or translation at an interface 462 between the passive catheter body and the actively driven portion.

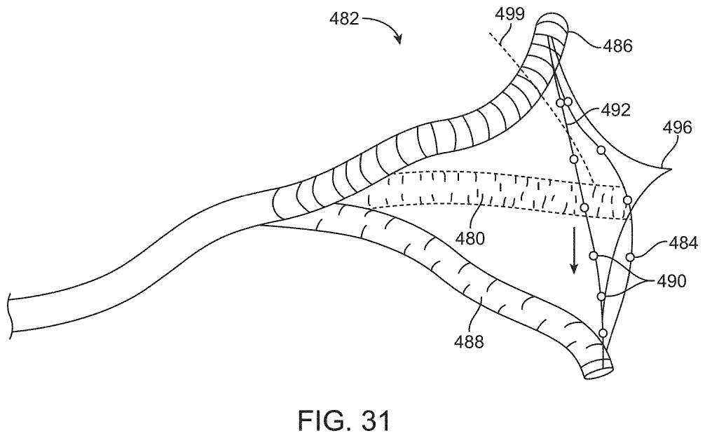

Referring now to FIGS. 30B and 30C, the articulated distal portion of the catheter can be articulated in response to the sensed rotational and/or axial movement so as to compensate for the movement of the interface 462 such that displacement of a distal tip 464 of the catheter within the patient in response to the movement of the interface is inhibited. Addressing roll about the catheter axis with reference to FIG. 30B, the articulated distal portion can include a proximal articulated segment 466 having a drive-alterable proximal curvature of axis 436 and a distal articulated segment 468 having a distal drive-alterable curvature of axis 436 with a segment interface 470 therebetween. When the manipulating of the proximal end of the catheter includes manually rotating the proximal end of the catheter about the axis of the catheter the articulating of the articulated distal portion can be performed so as to induce precessing 472 of the proximal curvature about the axis of the catheter adjacent the interface, optionally along with precessing 474 of the distal curvature about the axis of the catheter adjacent the segment interface such that lateral displacement of the distal tip of the catheter in response to the manual rotation of the catheter is inhibited. Manual rotation from outside the body with a fixed catheter tip inside the body can be particularly helpful for rotation of a tool supported adjacent the tip into a desired orientation about the axis of the catheter relative to a target tissue. Addressing manual movement along the catheter axis with reference to FIG. 30C, the articulated distal portion can similarly include a proximal articulated segment having a proximal curvature and a distal articulated segment having a distal curvature with a segment interface therebetween (see FIG. 30B). The articulating of the articulated distal portion can be performed so as to induce a first change in the proximal curvature and a second change in the distal curvature such that axial displacement of the distal tip of the catheter in response to the manual displacement of the catheter is inhibited, which can be useful for re-positioning a workspace 480 of a tool adjacent the distal tip of the catheter so as to encompass a target tissue. [0337] Referring now to FIG. 31, a virtual trajectory verification image 480 of the catheter can be included in the virtual and/or hybrid workspace image 482 to allow a user to visually review a proposed movement of the actual catheter along a trajectory 484 from a current catheter image 486 to a desired or phantom catheter image 488. To generate the trajectory 484, the processor may identify a plurality of verification locations 490 along an initial candidate trajectory 492 (such as a straight-line trajectory). The processor may seek to calculate drive signals for the verification locations using the methods described above, and for any of the verification locations outside a workspace boundary 494 of the catheter, the processor can identify alternative verification locations within the workspace. Smoothing the initial alternative path 496 between the alternative verifications locations can help provide a more desirable smoothed path to be used as the trajectory 484. Optionally, the current and desired locations may be identified in response to receipt, by the processor, of a command to go back to a prior pose of the catheter, with the desired pose comprising the prior pose and the catheter having moved from the prior pose along a previous trajectory,

While the exemplary embodiments have been described in some detail for clarity of understanding and by way of example, a variety of modifications, changes, and adaptations of the structures and methods described herein will be obvious to those of skill in the art. Hence, the scope of the present invention is limited solely by the claims attached hereto.

1. A method for aligning a therapeutic or diagnostic tool with a target tissue adjacent an internal site in a patient, using an elongate body inserted into the patient, the elongate body having a receptacle to support the tool and the receptacle defining a first pose within the internal surgical site, the method comprising:
receiving, with a processor of a surgical robotic system and from a user, input for moving an image of the receptacle from the first pose to a second pose within the internal surgical site;
receiving, with the processor, a movement command to move the receptacle; and
transmitting, from the processor in response to the movement command, drive signals to a plurality of actuators so as to advance the receptacle along a trajectory from the first pose toward the second pose.

2. The method of claim 1, wherein the input defines an intermediate input pose after the first pose and before the second pose, and wherein the trajectory is independent of the intermediate input pose;
wherein the movement command comprises a command to move along an incomplete spatial portion of a trajectory from the first pose to the second pose and to stop at an intermediate pose between the first pose and the second pose; and
wherein the processor transmits, in response to the movement command, the drive signals so as to move the receptacle toward the intermediate pose.

3.-21. (canceled)

22. A method for presenting an image to a user of a target tissue of a patient body, the method comprising:
receiving a first two-dimensional (2D) image dataset, the first 2D dataset defining a first image including the target tissue and a tool receptacle of a tool delivery system disposed within the patient body, the first image having a first orientation relative to the receptacle;
receiving a second 2D image dataset defining a second target image including the target tissue and the tool delivery system, the second image having a second orientation relative to the receptacle, the second orientation angularly offset from the first orientation;
transmitting hybrid 2D/three-dimensional (3D) image data to a display device so as to present a hybrid 2D/3D image for reference by the user, the hybrid image comprising 2D image components in a 3D image space and including:
the first 2D image with the first orientation relative to a 3D model of the tool delivery system; and
the second 2D image having the second orientation relative to the 3D model, the first and second 2D images positionally offset from the model.

23. The method of claim 22, wherein the hybrid image comprises a 3D virtual image of the model, the model comprising a calculated virtual pose of the receptacle;
wherein the first 2D image is disposed on a first plane in the hybrid image, the first plane being offset from the model along a first normal to the first plane; and/or
wherein the second 2D image is disposed on a second plane in the hybrid image, the second plane being offset from the model along a second normal to the second plane.

24. (canceled)

25. The method of claim 23, wherein the hybrid image includes a first 2D virtual image of the model superimposed on the first 2D image, the first 2D virtual image being at the first orientation relative to the model; and/or
wherein the hybrid image includes a second 2D virtual image of the model superimposed on the second 2D image, the second 2D virtual image being at the second orientation relative to the model;
wherein the model includes a phantom defining a phantom receptacle pose angularly and/or positionally offset from the virtual receptacle pose, wherein the 3D virtual image includes the phantom, and wherein the hybrid image includes a first 2D augmented image of the phantom with the first orientation superimposed on the first 2D image, and further comprising:
receiving a movement command from a hand of the user to move relative to the display;
moving the phantom pose in correlation with the movement command;
displaying the moved phantom on the first 2D image and the second 2D image; and
calculating a trajectory between the virtual tool and the phantom and moving the tool within the patient body by articulating an elongate body supporting the tool in response to a one-dimensional (1D) input from the user.

26. (canceled)

27. The method of claim 25, further comprising:
constraining motion relative to the first plane so that an image of the receptacle moves:
along the first plane; or
normal to the first plane.

28. The method of claim 22, wherein the first 2D image comprises a substantially real-time video image, and wherein the second 2D image comprises a recorded image of the target tissue and the tool system.

29. The method of claim 22, wherein the first and second 2D images comprise ultrasound or fluoroscope images of the target tissue and the tool system.

30. A method for presenting an image to a user of a target tissue of a patient body on a display device having a display plane, the method comprising:
receiving a first two-dimensional (2D) image dataset, the first 2D dataset defining a first image including the target tissue and a tool receptacle of a tool delivery system disposed within the patient body, the first image having a first orientation relative to the receptacle;
transmitting hybrid 2D/three-dimensional (3D) image data to the display device so as to present a hybrid 2D/3D image for reference by the user, the hybrid image including:
the first 2D image with the first orientation relative to a 3D model of the tool delivery system; and
a 3D image the 3D model;
wherein the first 2D image is orientationally offset relative to the display plane of the display device.

31. (canceled)

32. A method for moving a tool of a tool delivery system in a patient body with reference to a display image shown on a display, the display image showing a target tissue and the tool and defining a display coordinate system, the tool delivery system including an articulated elongate body coupled with the tool and having 3 or more degrees of freedom, the method comprising:
determining, in response to a movement command entered by a hand of a user relative to the display image, a desired movement of the tool;
calculating, in response to the movement command, an articulation of the elongate body so as to move the tool within the patient body, wherein the calculation of the articulation is performed by constraining the tool relative to a first plane of the display coordinate system so that the image of the tool moves:
along the first plane; or
normal to the first plane; and
transmitting the calculated articulation so as to effect movement of the tool.

33. The method of claim 32, further comprising receiving a first two-dimensional (2D) image dataset, the first 2D dataset defining a first image showing the target tissue and the tool, the first image being along the first plane, wherein image data corresponding to the first 2D image dataset is transmitted to the to the display device so as to generate the display image;
wherein the display coordinate frame includes a view plane extending along a surface of the display, and wherein the first plane is angularly offset from the view plane.

34. (canceled)

35. The method of claim 33, further comprising identifying the first plane in response to a plane command from the user.

36. The method of claim 35, wherein the first image plane has a first orientation relative to the tool, and further comprising:
receiving a second 2D image dataset defining a second target image showing the target tissue and the tool delivery system, the second image having a second orientation relative to the receptacle, the second orientation angularly offset from the first orientation;
transmitting the image data to the display, the image data comprising hybrid 2D/three-dimensional (3D) image data and the display presenting a hybrid image for reference by the user, the hybrid image showing:
the first 2D image with the first orientation relative to a 3D model of the tool delivery system; and
the second 2D image having the second orientation relative to the 3D model, the first and second 2D images positionally offset from the model.

37. The method of claim 32, further comprising sensing the movement command in 3 or more degrees of freedom.

38. The method of claim 32, further comprising sensing the movement command in 6 degrees of freedom, wherein the calculated movement command in a first mode effects:
translation of the tool along the plane first plane; and
rotation of the tool about an axis normal to the first plane; and
wherein the calculated movement command in a second mode effects:
translation of the tool normal to the first plane; and
rotation of the tool about an axis parallel to the first plane and normal to an axis of the tool.

39. The method of claim 38, wherein the tool system comprises a phantom and the display image comprises an augmented reality image with a phantom image and another image of the tool receptacle, and wherein the movement command in a third mode effects movement of the receptacle along a trajectory between the phantom image and the other image.

40. The method of claim 32, wherein the tool delivery system has a plurality of degrees of freedom, further comprising limiting the calculated articulation so that the receptacle is constrained to movement along a spatial construct, wherein a workspace boundary is disposed with the patient body between a current position of the receptacle and a desired position of the receptacle defined by the movement command, and further comprising determining the calculated articulation so as to induce movement of the receptacle along the spatial construct to adjacent the boundary.

41. The method of claim 40, wherein the constrained movement is selected from the group consisting of translation in 3D space, movement along a plane, movement along a line, gimbal rotation about a plurality of intersecting axes, and rotation about an axis.

42. The method of claim 32, wherein a workspace boundary is disposed between a location of the tool before the commanded movement and a desired location of the tool defined by the commanded movement, and further comprising limiting the movement to movement along a spatial construct by generating a plurality of test solutions for test movement commands at test poses of the tool along the construct, determining a plurality of command gradients from the test solutions, and generating the movement command from the test poses and command gradients so that the commanded movement induces movement of the tool along the construct and within the workspace to adjacent the boundary.

43.-44. (canceled)

45. The method of claim 30, further comprising graphically indicating an orientation of the first 2D image dataset relative to the patient body and the offset orientation of the display plane relative to the first 2D dataset.

46.-49. (canceled)

50. An image-guided therapy method for treating a patient body, the method comprising:
generating a three-dimensional (3D) virtual therapy workspace inside the patient body and a three-dimensional (3D) virtual image of a therapy tool within the 3D virtual workspace; and
aligning an actual 2D image of the tool in the patient body with the 3D virtual image, the actual image having an image plane;
superimposing the actual image with the 3D virtual image so as to generate a hybrid image; and
transmitting the hybrid image to a display having a display plane so as to present the hybrid image with the image plane of the actual image at an angle relative to the display plane.

51.-56. (canceled)

57. A method for driving a medical robotic system, the system configured for manipulating a tool receptacle in a workspace within a patient body with reference to a display, the receptacle defining a first pose in the workspace and the display showing a workspace image of the receptacle and/or a tool supported thereby in the workspace, the method comprising:
receiving input, with a processor and relative to the workspace image, defining an input trajectory from the first pose to a desired pose of the receptacle and/or tool within the workspace;
calculating, with the processor, a candidate trajectory from the first pose to the desired pose; and
transmitting drive commands from the processor in response to the candidate trajectory so as to induce movement of the tool and/or receptacle toward the desired pose.

58. The method of claim 57, the workspace image including a tissue image of tissue adjacent the workspace, the tool and/or receptacle being supported by an elongate flexible catheter having an image shown on the display and further comprising superimposing, on the display:
a phantom catheter with the desired pose; and
a trajectory validation catheter between initial pose and the desired pose to facilitate visual validation of catheter movement safety prior to transmitting of the drive commands.

59. The method of claim 58, further comprising:
identifying a plurality of verification locations along the candidate trajectory; and
for any of the verification locations outside a workspace of the catheter, identifying alternative verification locations within the workspace and smoothing a path in response to the verification locations and any alternative verification locations;
wherein superimposing the validation catheter is performed by advancing the validation catheter between the verification locations and any alternative verification locations.

60. The method of claim 58, wherein the first location was identified in response to receipt, by the processor, of a command to go back to a prior pose of the catheter, the desired pose comprising the prior pose and the catheter having moved from the prior pose along a previous trajectory,

61. (canceled)
